(19) United States
(12) Patent Application Publication
(10) Pub. No.: US 2025/0171542 A1
(43) Pub. Date: May 29, 2025

(54) ANTI-PROGRAMMED DEATH-LIGAND 1 (PD-L1) ANTIBODIES

(71) Applicants: Adept Therapeutics Inc., Beverly, MA (US); Adept Biopharmaceutical and Technology, Ltd., Shenzhen (CN)

(72) Inventors: Xinyan Zhao, Bedford, MA (US); Changyun Hu, Belmont, MA (US); Yang Lou, Worcester, MA (US); Haibin Huang, Worcester, MA (US)

(21) Appl. No.: 18/841,758

(22) PCT Filed: Feb. 28, 2023
(86) PCT No.: PCT/US2023/014084

§ 371 Date: Aug. 27, 2024

Related U.S. Application Data
(60) Provisional application No. 63/314,925, filed on Feb. 28, 2022.

(51) Int. Cl. C07K 16/28; A61K 47/68

(52) U.S. Cl. CPC C07K 16/2827; A61K 47/68037; A61K 47/6849; C07K 2317/33; C07K 2317/565; C07K 2317/732; C07K 2317/76; C07K 2317/77; C07K 2317/92

(57) ABSTRACT

The present invention provides anti-PD-L1 antigen binding molecules, including antibodies and the antigen binding fragment thereof, and methods for using the same for treating a variety of diseases, including cancers. The antibodies may be delivered to a subject within a pharmaceutical composition for treatment of a variety of diseases, including various types of cancers. Administration results in an activated immune system that can then target tumor cells within the subject. Additional therapeutic agents, including other pharmaceuticals, may be administered in conjunction with the molecules of the invention to aid the treatment of the subject.

### CROSS-REFERENCE TO RELATED APPLICATIONS

The instant application is a 35 U.S.C. § 371 national stage filing of International Application No. PCT/US2023/014084, filed on Feb. 28, 2023, which claims priority to U.S. Provisional Application No. 63/314,925, filed on Feb. 28, 2022. The entire contents of the foregoing applications are incorporated herein by reference.

### STATEMENT REGARDING SEQUENCE LISTING

The instant application contains a Sequence Listing which has been submitted electronically in .XML format and is hereby incorporated by reference in its entirety. Said .XML copy, created on Nov. 1, 2023, is named 136546-00202_Sequence_Listing.xml and is 432,061 bytes in size. The sequence listing contained in this .XML file is part of the specification and is hereby incorporated by reference herein in its entirety.

### FIELDS

The present invention relates to antigen binding molecules, e.g., antibodies or antigen binding fragments thereof, for cancer treatment and, in particular, to anti-programmed death-ligand 1 (PD-L1) antibodies for cancer immunotherapy.

### BACKGROUND

Adaptive immune responses are mediated by two major subsets of immune cells: T lymphocytes (CD4⁺ T cells and CD8⁺ T cells) and B lymphocytes. While B cells play major roles in secreting antibodies and acting as antigen-presenting cells, T cells have been extensively explored for the extraordinary effector functions during various physiological responses, including acute and chronic infection, cancer and autoimmunity, and in immune homeostasis. T cell activation requires two distinct signals, ligation via peptide-MHC engagement of T cell receptor (TCR) and positive co-stimulatory signals such as interactions between CD28 on T cells and CD80 (also known as B7.1) and/or CD86 (also known as B7.2) on antigen-presenting cells. Early during the activation process, negative regulators including Cytotoxic T lymphocyte antigen 4 (CTLA4; also known as CD152) are induced to counteract the activation programme and competes with CD28 for the ligands CD80 and CD86. Programmed cell death protein 1 (PD1, also known as PDCD1 and CD279) is also expressed during T cell activation and counters positive signals through the TCR and CD28 by engaging its ligands programmed cell death 1 ligand 1 (PD-L1; also known as CD274 and B7-H1) and/or PD-L2 (also known as CD273 and B7-DC).

PD-L1 is a 53 kDa type 1 transmembrane protein, expressed by many immune cell subsets including activated and exhausted/anergic T cells, B cells, and myeloid cells including monocyte, mast cells and dendritic cells, and some cancer cells. It is one of the ‘coinhibitory’ receptors that function as breaks for the adaptive immune responses, serving as immune checkpoints that effector T cells must pass in order to exert their full functions. Under pathologic conditions, expression of PD-L1 is found to be significantly upregulated in many tumors, including but not limited to non-small cell lung cancer (NSCLC), bladder cancer, hepatocellular cancer, breast cancer, pancreatic cancer. The increased expression of PD-L1 on tumor cells can modulate tumor microenvironment (TME) to promote immune evasion of tumor cells through 1) enhancing immunosuppression via stimulating regulatory T cells, and 2) reducing inflammation by driving T cell exhaustion/anergy.

Treatment with PD1 pathway inhibitors (known as ‘checkpoint blockade’) showed success in promoting durable antitumor immune responses, and this success led to the approval by the US Food and Drug Administration of the monoclonal antibodies Pembrolizumab (anti-PD1; Merck & Co), Nivolumab (anti-PD1; Bristol Myers Squibb), Atezolizumab (anti-PDL1; Roche/Genentech), and Durvalumab (anti-PDL1; AstraZeneca), Avelumab (anti-PDL1; EMD Serono), Cemiplimab (anti-PD-1; Regeneron), Dostarlimab (anti-PD-1, GlaxoSmithKline) for therapeutic use in more than 20 cancer indications, including melanoma, bladder cancer, non-small-cell lung cancer (NSCLC), hepatocellular carcinoma (HCC), renal cell carcinoma (RCC), head and neck squamous cell carcinoma (HNSC), Hodgkin lymphoma, Merkel cell carcinoma and microsatellite instability high or mismatch repair-deficient adult and pediatric solid tumors (Ref. Cancer research Institute, https://www.cancerresearch.org/fda-approval-timeline-of-active-immunotherapies).

Despite the revolutionary cure-like survival benefit observed in trials, only a minority of cancer patients are estimated to experience a positive response to PD-1/PD-L1 blockade therapy, and the primary or acquired resistance eventually could lead to cancer progression in patients with clinical responses. To overcome the limitation in therapy resistance, substantial effort has been made to improve or develop novel anti-PD-1/PD-L1 based immunotherapy strategies with better clinical response and reduced immune-mediated toxicity. There is a need in the art for novel therapies based on novel antigen binding molecules, e.g., antibodies or antigen binding fragments thereof, that specifically binds to PD-L1 for use in the treatment of diseases, e.g., cancers.

### SUMMARY

The present invention provides antigen binding molecules, e.g., anti-PD-L1 antibodies or antigen binding fragments thereof, for binding, e.g., specifically binding, to PD-L1. The PD-L1 may be on the surface of a cell, e.g., a mammalian cell, such as a tumor cell of a mammal, e.g., a mouse tumor cell, a cynomolgus tumor cell or a human tumor cell. The present invention also provides methods of using the antigen binding molecules, e.g., anti-PD-L1 antibodies or antigen binding fragments thereof, of the present invention, for specifically binding to PD-L1. The binding of the antigen binding molecules, e.g., anti-PD-L1 antibodies or antigen binding fragments thereof, of the present invention, may inhibit the PD-1/PD-L1 interaction or induce the internalization of the complex of PD-L1 and the antigen binding molecule. The binding of the antigen binding molecules, e.g., anti-PD-L1 antibodies or antigen binding fragments thereof, of the present invention, can be used for treating a subject who would benefit from modulating, e.g., inhibiting, the PD-1/PD-L1 interaction, e.g., a subject suffering or prone to suffering from a PD-L1-associated disease, e.g. a disease or disorder characterized by abnormal expression of PD-L1.

Accordingly, in one aspect, the present invention provides an isolated antigen binding molecule, e.g., an antibody or antigen-binding fragment thereof, that binds to human PD-L1. The antibody includes a heavy chain variable (VH) domain comprising from N-terminus to C-terminus, three heavy chain complementarity-determining regions (CDRs), HCDR1, HCDR2, and HCDR3; and a light chain variable (VL) domain comprising from N-terminus to C-terminus, three light chain complementarity-determining regions (CDRs), LCDR1, LCDR2, and LCDR3; wherein (a) the HCDR1 comprises an amino acid sequence selected from the group consisting of X1-N-H-Y-M-X2 (SEQ ID NO: 301), X3-X4-Y-Y-M-C (SEQ ID NO: 303), N-N-Y-Y-M-S (SEQ ID NO: 8), R-Y-F-Y-M-S (SEQ ID NO: 11), and S-A-Y-W-I-C (SEQ ID NO: 12); wherein X1 is N or S, X2 is C or S, X3 is A, N, or S, and X4 is A, N, or S; (b) the HCDR2 comprises an amino acid sequence X32-I-X33-X34-G-S-X35-I-X36-D-Y-A-X37-W-A-K-G (SEQ ID NO: 226), wherein X32 is C or S, X33 is G or S, X34 is I, T, or V, X35 is A, D, G, or Y, X36 is S or T, and X37 is N or S; (c) the HCDR3 comprises an amino acid sequence WTSGGGGFGL (SEQ ID NO: 42); (d) the LCDR1 comprises an amino acid sequence selected from the group consisting of Q-S-S-Q-X65-X66-Y-X67-X68-Y-L-X69 (SEQ ID NO: 239), Q-S-S-Q-X70-I-Y-S-D-Y-L-X71 (SEQ ID NO: 240), and Q-S-S-Q-S-X72-Y-X73-N-Y-L-X74 (SEQ ID NO: 241); wherein X65 is N, S, or T, X66 is I or V, X67 is N or S, X68 is D or N, and X69 is A, C, F, or S, X70 is N or T, X71 is F or S, X72 is I or V, X73 is N or S, X74 is A, C, or S; (e) the LCDR2 comprises an amino acid sequence selected from the group consisting of X98-X99-X100-T-L-A-S (SEQ ID NO: 256), X101-X102-S-T-L-A-S (SEQ ID NO: 257), and S-T-A-T-L-A-S (SEQ ID NO: 72); wherein X98 is D, G, Q, S, or Y, X99 is A or T, X100 is A or S, X101 is D, G, Q, or Y, X102 is A or T; and (f) the LCDR3 comprises an amino acid sequence Q-G-Y-Y-S-G-Y-I-W-T (SEQ ID NO: 83).

In various aspects of the invention and embodiments thereof, the antibody is an antigen binding fragment of the antibody. In various aspects of the invention and embodiments thereof, the human PD-L1 comprises a sequence as set forth in SEQ ID NO: 312.

In one embodiment, (a) the HCDR1 comprises an amino acid sequence selected from the group consisting of SEQ ID NOs: 1-12; (b) the HCDR2 comprises an amino acid sequence selected from the group consisting of SEQ ID NOs: 22-31; (c) the HCDR3 comprises an amino acid sequence as set forth in SEQ ID NO: 42; (d) the LCDR1 comprises an amino acid sequence selected from the group consisting of SEQ ID NOs: 53-59; (e) the LCDR2 comprises an amino acid sequence selected from the group consisting of SEQ ID NOs: 69-73; and (f) the LCDR3 comprises an amino acid sequence as set forth in SEQ ID NO: 83.

In another embodiment, the isolated antigen binding molecule, e.g., the antibody or antigen-binding fragment thereof, includes the HCDR1 comprising an amino acid sequence set forth in SEQ ID NO: 1, the HCDR2 comprising an amino acid sequence set forth in SEQ ID NO: 22, the HCDR3 comprising an amino acid sequence set forth in SEQ ID NO: 42, the LCDR1 comprising an amino acid sequence set forth in SEQ ID NO: 53, the LCDR2 comprising an amino acid sequence set forth in SEQ ID NO: 69, and the LCDR3 comprising an amino acid sequence set forth in SEQ ID NO: 83; (b) the HCDR1 comprising an amino acid sequence set forth in SEQ ID NO: 2, the HCDR2 comprising an amino acid sequence set forth in SEQ ID NO: 23, the HCDR3 comprising an amino acid sequence set forth in SEQ ID NO: 42, the LCDR1 comprising an amino acid sequence set forth in SEQ ID NO: 53, the LCDR2 comprising an amino acid sequence set forth in SEQ ID NO: 69, and the LCDR3 comprising an amino acid sequence set forth in SEQ ID NO: 83; (c) the HCDR1 comprising an amino acid sequence set forth in SEQ ID NO: 3, the HCDR2 comprising an amino acid sequence set forth in SEQ ID NO: 23, the HCDR3 comprising an amino acid sequence set forth in SEQ ID NO: 42, the LCDR1 comprising an amino acid sequence set forth in SEQ ID NO: 54, the LCDR2 comprising an amino acid sequence set forth in SEQ ID NO: 70, and the LCDR3 comprising an amino acid sequence set forth in SEQ ID NO: 83; (d) the HCDR1 comprising an amino acid sequence set forth in SEQ ID NO: 4, the HCDR2 comprising an amino acid sequence set forth in SEQ ID NO: 24, the HCDR3 comprising an amino acid sequence set forth in SEQ ID NO: 42, the LCDR1 comprising an amino acid sequence set forth in SEQ ID NO: 55, the LCDR2 comprising an amino acid sequence set forth in SEQ ID NO: 71, and the LCDR3 comprising an amino acid sequence set forth in SEQ ID NO: 83; (e) the HCDR1 comprising an amino acid sequence set forth in SEQ ID NO: 5, the HCDR2 comprising an amino acid sequence set forth in SEQ ID NO: 25, the HCDR3 comprising an amino acid sequence set forth in SEQ ID NO: 42, the LCDR1 comprising an amino acid sequence set forth in SEQ ID NO: 56, the LCDR2 comprising an amino acid sequence set forth in SEQ ID NO: 69, and the LCDR3 comprising an amino acid sequence set forth in SEQ ID NO: 83; (f) the HCDR1 comprising an amino acid sequence set forth in SEQ ID NO: 6, the HCDR2 comprising an amino acid sequence set forth in SEQ ID NO: 26, the HCDR3 comprising an amino acid sequence set forth in SEQ ID NO: 42, the LCDR1 comprising an amino acid sequence set forth in SEQ ID NO: 57, the LCDR2 comprising an amino acid sequence set forth in SEQ ID NO: 71, and the LCDR3 comprising an amino acid sequence set forth in SEQ ID NO: 83; (g) the HCDR1 comprising an amino acid sequence set forth in SEQ ID NO: 7, the HCDR2 comprising an amino acid sequence set forth in SEQ ID NO: 27, the HCDR3 comprising an amino acid sequence set forth in SEQ ID NO: 42, the LCDR1 comprising an amino acid sequence set forth in SEQ ID NO: 56, the LCDR2 comprising an amino acid sequence set forth in SEQ ID NO: 69, and the LCDR3 comprising an amino acid sequence set forth in SEQ ID NO: 83; (h) the HCDR1 comprising an amino acid sequence set forth in SEQ ID NO: 8, the HCDR2 comprising an amino acid sequence set forth in SEQ ID NO: 28, the HCDR3 comprising an amino acid sequence set forth in SEQ ID NO: 42, the LCDR1 comprising an amino acid sequence set forth in SEQ ID NO: 53, the LCDR2 comprising an amino acid sequence set forth in SEQ ID NO: 69, and the LCDR3 comprising an amino acid sequence set forth in SEQ ID NO: 83; (i) the HCDR1 comprising an amino acid sequence set forth in SEQ ID NO: 9, the HCDR2 comprising an amino acid sequence set forth in SEQ ID NO: 27, the HCDR3 comprising an amino acid sequence set forth in SEQ ID NO: 42, the LCDR1 comprising an amino acid sequence set forth in SEQ ID NO: 53, the LCDR2 comprising an amino acid sequence set forth in SEQ ID NO: 69, and the LCDR3 comprising an amino acid sequence set forth in SEQ ID NO: 83; (j) the HCDR1 comprising an amino acid sequence set forth in SEQ ID NO: 5, the HCDR2 comprising an amino acid sequence set forth in SEQ ID NO: 29, the HCDR3 comprising an amino acid sequence set forth in SEQ ID NO: 42, the LCDR1 comprising an amino acid sequence set forth in SEQ ID NO: 53, the LCDR2 comprising an amino acid sequence set forth in SEQ ID NO: 69, and the LCDR3 comprising an amino acid sequence set forth in SEQ ID NO: 83; (k) the HCDR1 comprising an amino acid sequence set forth in SEQ ID NO: 11, the HCDR2 comprising an amino acid sequence set forth in SEQ ID NO: 30, the HCDR3 comprising an amino acid sequence set forth in SEQ ID NO: 42, the LCDR1 comprising an amino acid sequence set forth in SEQ ID NO: 58, the LCDR2 comprising an amino acid sequence set forth in SEQ ID NO: 72, and the LCDR3 comprising an amino acid sequence set forth in SEQ ID NO: 83; or (l) the HCDR1 comprising an amino acid sequence set forth in SEQ ID NO: 12, the HCDR2 comprising an amino acid sequence set forth in SEQ ID NO: 31, the HCDR3 comprising an amino acid sequence set forth in SEQ ID NO: 42, the LCDR1 comprising an amino acid sequence set forth in SEQ ID NO: 59, the LCDR2 comprising an amino acid sequence set forth in SEQ ID NO: 73, and the LCDR3 comprising an amino acid sequence set forth in SEQ ID NO: 83.

In still another embodiment, the isolated antigen binding molecule, e.g., the antibody or antigen-binding fragment thereof, includes (a) a heavy chain variable region (HCVR) comprising an amino acid sequence selected from the group consisting of SEQ ID NOs: 92-103; and (b) a light chain variable region (LCVR) comprising an amino acid sequence selected from the group consisting of SEQ ID NOs: 118-129.

In yet another embodiment, the isolated antigen binding molecule, e.g., the antibody or antigen-binding fragment thereof, includes (a) the HCVR comprising an amino acid sequence set forth in SEQ ID NO: 92, and the LCVR comprising an amino acid sequence set forth in SEQ ID NO: 118; (b) the HCVR comprising an amino acid sequence set forth in SEQ ID NO: 93, and the LCVR comprising an amino acid sequence set forth in SEQ ID NO: 119; (c) the HCVR comprising an amino acid sequence set forth in SEQ ID NO: 94, and the LCVR comprising an amino acid sequence set forth in SEQ ID NO: 120; (d) the HCVR comprising an amino acid sequence set forth in SEQ ID NO: 95, and the LCVR comprising an amino acid sequence set forth in SEQ ID NO: 121; (e) the HCVR comprising an amino acid sequence set forth in SEQ ID NO: 96, and the LCVR comprising an amino acid sequence set forth in SEQ ID NO: 122; (f) the HCVR comprising an amino acid sequence set forth in SEQ ID NO: 97, and the LCVR comprising an amino acid sequence set forth in SEQ ID NO: 123; (g) the HCVR comprising an amino acid sequence set forth in SEQ ID NO: 98, and the LCVR comprising an amino acid sequence set forth in SEQ ID NO: 124; (h) the HCVR comprising an amino acid sequence set forth in SEQ ID NO: 99, and the LCVR comprising an amino acid sequence set forth in SEQ ID NO: 125; (i) the HCVR comprising an amino acid sequence set forth in SEQ ID NO: 100, and the LCVR comprising an amino acid sequence set forth in SEQ ID NO: 126; (j) the HCVR comprising an amino acid sequence set forth in SEQ ID NO: 101, and the LCVR comprising an amino acid sequence set forth in SEQ ID NO: 127; (k) the HCVR comprising an amino acid sequence set forth in SEQ ID NO: 102, and the LCVR comprising an amino acid sequence set forth in SEQ ID NO: 128; or (l) the HCVR comprising an amino acid sequence set forth in SEQ ID NO: 103, and the LCVR comprising an amino acid sequence set forth in SEQ ID NO: 129.

In one aspect, the present invention provides an isolated antigen binding molecule, e.g., an antibody or antigen-binding fragment thereof, that binds to human PD-L1. The isolated antigen binding molecule, e.g., the antibody or antigen-binding fragment thereof, includes a heavy chain variable (VH) domain comprising from N-terminus to C-terminus, three heavy chain complementarity-determining regions (CDRs), HCDR1, HCDR2, and HCDR3; and a light chain variable (VL) domain comprising from N-terminus to C-terminus, three light chain complementarity-determining regions (CDRs), LCDR1, LCDR2, and LCDR3; wherein (a) the HCDR1 comprises an amino acid sequence that is about 80%, 85%, 90%, 95%, 96%, 97%, 98%, 99% to about 100% identical to an amino acid sequence selected from the group consisting of SEQ ID NOs: 1-12; (b) the HCDR2 comprises an amino acid sequence that is about 80%, 85%, 90%, 95%, 96%, 97%, 98%, 99% to about 100% identical to an amino acid sequence selected from the group consisting of SEQ ID NOs: 22-31; (c) the HCDR3 comprises an amino acid sequence that is about 80%, 85%, 90%, 95%, 96%, 97%, 98%, 99% to about 100% identical to an amino acid sequence set forth in SEQ ID NO: 42; (d) the LCDR1 comprises an amino acid sequence that is about 80%, 85%, 90%, 95%, 96%, 97%, 98%, 99% to about 100% identical to an amino acid sequence selected from the group consisting of SEQ ID NOs: 53-59; (e) the LCDR2 comprises an amino acid sequence that is about 80%, 85%, 90%, 95%, 96%, 97%, 98%, 99% to about 100% identical to an amino acid sequence selected from the group consisting of SEQ ID NOs: 69-73, (f) the LCDR3 comprises an amino acid sequence that is about 80%, 85%, 90%, 95%, 96%, 97%, 98%, 99% to about 100% identical to an amino acid sequence as set forth in SEQ ID NO: 83.

In another aspect, the present invention provides an isolated antigen binding molecule, e.g., an antibody or the antigen binding fragment thereof, that binds human PD-L1. The isolated antigen binding molecule, e.g., the antibody or antigen-binding fragment thereof includes (a) a heavy chain variable region (HCVR) comprising an amino acid sequence that is about 80%, 85%, 90%, 95%, 96%, 97%, 98%, 99% to about 100% identical to an amino acid sequence selected from the group consisting of SEQ ID NOs: 92-103; and (b) a light chain variable region (LCVR) comprising an amino acid sequence that is about 80%, 85%, 90%, 95%, 96%, 97%, 98%, 99% to about 100% identical to an amino acid sequence selected from the group consisting of SEQ ID NOs: 118-129.

In still another aspect, the present invention provides an isolated antigen binding molecule, e.g., an antibody or the antigen binding fragment thereof, that binds human PD-L1. The isolated antigen binding molecule, e.g., the antibody or antigen-binding fragment thereof includes a heavy chain variable (V_{H}) domain comprising from N-terminus to C-terminus, three heavy chain complementarity-determining regions (CDRs), HCDR1, HCDR2, and HCDR3; and a light chain variable (V_{L}) domain comprising from N-terminus to C-terminus, three light chain complementarity-determining regions (CDRs), LCDR1, LCDR2, and LCDR3; wherein the HCDR1 comprises an amino acid sequence X8-D-X9-Y-M-S (SEQ ID NO: 306); wherein X8 is D or G, and X9 is W or Y; (b) the HCDR2 comprises an amino acid sequence S-I-Y-X42-G-S-L-N-X43-Y-Y-A-T-W-A-K-G (SEQ ID NO: 229), wherein X42 is S or T, and X43 is I, S or T; (c) the HCDR3 comprises an amino acid sequence R-X57-K-N-X58-D-X59-G-X60-F-D-L (SEQ ID NO: 237), wherein X57 is H, N, or T, X58 is A or G, X59 is W or Y, and X60 is H or Y; (d) the LCDR1 comprises an amino acid sequence selected from the group consisting of Q-A-S-X76-S-I-X77-X78-X79-L-A (SEQ ID NO: 243), Q-A-S-X80-S-I-N-D-R-L-A (SEQ ID NO: 244), Q-A-S-Q-S-I-X81-X82-A-L-A (SEQ ID NO: 245), Q-A-S-Q-S-I-S-T-A-L-A (SEQ ID NO: 67), and Q-A-S-Q-S-I-G-N-A-L-A (SEQ ID NO: 68), wherein X76 is E or Q, X77 is G, N, or S, X78 is D, N, or T, X79 is A or R, X80 is E or Q, X81 is G or S, and X82 is N or T; (e) the LCDR2 comprises an amino acid sequence X106-X107-S-T-L-A-S (SEQ ID NO: 260); wherein X106 is A or G, and X107 is A or T; and (f) the LCDR3 comprises an amino acid sequence Q-Q-G-W-T-V-S-S-L-X111-N-A (SEQ ID NO: 263), wherein X111 is D or E.

In one embodiment (a) the HCDR1 comprises an amino acid sequence selected from the group consisting of SEQ ID NOs: 19, 20, and 21; (b) the HCDR2 comprises an amino acid sequence selected from the group consisting of SEQ ID NOs: 38, 39, 40, and 41; (c) the HCDR3 comprises an amino acid sequence selected from the group consisting of SEQ ID NO: 49, 50, 51, and 52; (d) the LCDR1 comprises an amino acid sequence selected from the group consisting of SEQ ID NOs: 65, 66, 67, and 68; (e) the LCDR2 comprises an amino acid sequence selected from the group consisting of SEQ ID NOs: 80, 71, and 82; and (f) the LCDR3 comprises an amino acid sequence as set forth in SEQ ID NO: 90 or 91.

In another embodiment, the isolated antigen binding molecule, e.g., the antibody or antigen-binding fragment thereof, includes (a) the HCDR1 comprising an amino acid sequence set forth in SEQ ID NO: 19, the HCDR2 comprising an amino acid sequence set forth in SEQ ID NO: 38, the HCDR3 comprising an amino acid sequence set forth in SEQ ID NO: 49, the LCDR1 comprising an amino acid sequence set forth in SEQ ID NO: 65, the LCDR2 comprising an amino acid sequence set forth in SEQ ID NO: 80, and the LCDR3 comprising an amino acid sequence set forth in SEQ ID NO: 90; (b) the HCDR1 comprising an amino acid sequence set forth in SEQ ID NO: 19, the HCDR2 comprising an amino acid sequence set forth in SEQ ID NO: 39, the HCDR3 comprising an amino acid sequence set forth in SEQ ID NO: 50, the LCDR1 comprising an amino acid sequence set forth in SEQ ID NO: 66, the LCDR2 comprising an amino acid sequence set forth in SEQ ID NO: 71, and the LCDR3 comprising an amino acid sequence set forth in SEQ ID NO: 90; (c) the HCDR1 comprising an amino acid sequence set forth in SEQ ID NO: 20, the HCDR2 comprising an amino acid sequence set forth in SEQ ID NO: 39, the HCDR3 comprising an amino acid sequence set forth in SEQ ID NO: 51, the LCDR1 comprising an amino acid sequence set forth in SEQ ID NO: 67, the LCDR2 comprising an amino acid sequence set forth in SEQ ID NO: 80, and the LCDR3 comprising an amino acid sequence set forth in SEQ ID NO: 90; (d) the HCDR1 comprising an amino acid sequence set forth in SEQ ID NO: 19, the HCDR2 comprising an amino acid sequence set forth in SEQ ID NO: 39, the HCDR3 comprising an amino acid sequence set forth in SEQ ID NO: 52, the LCDR1 comprising an amino acid sequence set forth in SEQ ID NO: 66, the LCDR2 comprising an amino acid sequence set forth in SEQ ID NO: 80, and the LCDR3 comprising an amino acid sequence set forth in SEQ ID NO: 91; (e) the HCDR1 comprising an amino acid sequence set forth in SEQ ID NO: 19, the HCDR2 comprising an amino acid sequence set forth in SEQ ID NO: 40, the HCDR3 comprising an amino acid sequence set forth in SEQ ID NO: 49, the LCDR1 comprising an amino acid sequence set forth in SEQ ID NO: 66, the LCDR2 comprising an amino acid sequence set forth in SEQ ID NO: 71, and the LCDR3 comprising an amino acid sequence set forth in SEQ ID NO: 90; (f) the HCDR1 comprising an amino acid sequence set forth in SEQ ID NO: 21, the HCDR2 comprising an amino acid sequence set forth in SEQ ID NO: 41, the HCDR3 comprising an amino acid sequence set forth in SEQ ID NO: 49, the LCDR1 comprising an amino acid sequence set forth in SEQ ID NO: 68, the LCDR2 comprising an amino acid sequence set forth in SEQ ID NO: 82, and the LCDR3 comprising an amino acid sequence set forth in SEQ ID NO: 90; (g) the HCDR1 comprising an amino acid sequence set forth in SEQ ID NO: 19, the HCDR2 comprising an amino acid sequence set forth in SEQ ID NO: 39, the HCDR3 comprising an amino acid sequence set forth in SEQ ID NO: 49, the LCDR1 comprising an amino acid sequence set forth in SEQ ID NO: 66, the LCDR2 comprising an amino acid sequence set forth in SEQ ID NO: 80, and the LCDR3 comprising an amino acid sequence set forth in SEQ ID NO: 90; or (h) the HCDR1 comprising an amino acid sequence set forth in SEQ ID NO: 19, the HCDR2 comprising an amino acid sequence set forth in SEQ ID NO: 39, the HCDR3 comprising an amino acid sequence set forth in SEQ ID NO: 49, the LCDR1 comprising an amino acid sequence set forth in SEQ ID NO: 66, the LCDR2 comprising an amino acid sequence set forth in SEQ ID NO: 71, and the LCDR3 comprising an amino acid sequence set forth in SEQ ID NO: 90.

In another embodiment, the isolated antigen binding molecule, e.g., the antibody the antigen binding fragment thereof, includes (a) a heavy chain variable region (HCVR) comprising an amino acid sequence selected from the group consisting of SEQ ID NOs: 110-117; (b) a light chain variable region (LCVR) comprising an amino acid sequence selected from the group consisting of SEQ ID NOs: 136-143.

In still another embodiment, the isolated antigen binding molecule, e.g., the antibody or the antigen binding fragment thereof, includes (a) the HCVR comprising an amino acid sequence set forth in SEQ ID NO: 110, and the LCVR comprising an amino acid sequence set forth in SEQ ID NO: 136; (b) the HCVR comprising an amino acid sequence set forth in SEQ ID NO: 111, and the LCVR comprising an amino acid sequence set forth in SEQ ID NO: 137; (c) the HCVR comprising an amino acid sequence set forth in SEQ ID NO: 112, and the LCVR comprising an amino acid sequence set forth in SEQ ID NO: 138; (d) the HCVR comprising an amino acid sequence set forth in SEQ ID NO: 113, and the LCVR comprising an amino acid sequence set forth in SEQ ID NO: 139; (e) the HCVR comprising an amino acid sequence set forth in SEQ ID NO: 114, and the LCVR comprising an amino acid sequence set forth in SEQ ID NO: 140; (f) the HCVR comprising an amino acid sequence set forth in SEQ ID NO: 115, and the LCVR comprising an amino acid sequence set forth in SEQ ID NO: 141; (g) the HCVR comprising an amino acid sequence set forth in SEQ ID NO: 116, and the LCVR comprising an amino acid sequence set forth in SEQ ID NO: 142; or (h) the HCVR comprising an amino acid sequence set forth in SEQ ID NO: 117, and the LCVR comprising an amino acid sequence set forth in SEQ ID NO: 143.

In one aspect, the present invention provides an isolated antigen binding molecule, e.g., an antibody or antigen binding fragment thereof, that binds to human PD-L1. The isolated antigen binding molecule, e.g., the antibody or antigen-binding fragment thereof, includes a heavy chain variable (VH) domain comprising from N-terminus to C-terminus, three heavy chain complementarity-determining regions (CDRs), HCDR1, HCDR2, and HCDR3; and a light chain variable (VL) domain comprising from N-terminus to C-terminus, three light chain complementarity-determining regions (CDRs), LCDR1, LCDR2, and LCDR3; wherein (a) the HCDR1 comprises an amino acid sequence that is about 80%, 85%, 90%, 95%, 96%, 97%, 98%, 99% to about 100% identical to an amino acid sequence selected from the group consisting of SEQ ID NOs: 19, 20, and 21; (b) the HCDR2 comprises an amino acid sequence that is about 80%, 85%, 90%, 95%, 96%, 97%, 98%, 99% to about 100% identical to an amino acid sequence selected from the group consisting of SEQ ID NOs: 38, 39, 40, and 41; (c) the HCDR3 comprises an amino acid sequence that is about 80%, 85%, 90%, 95%, 96%, 97%, 98%, 99% to about 100% identical to an amino acid sequence selected from the group consisting of: 49, 50, 51, and 52; (d) the LCDR1 comprises an amino acid sequence that is about 80%, 85%, 90%, 95%, 96%, 97%, 98%, 99% to about 100% identical to an amino acid sequence selected from the group consisting of SEQ ID NOs: 65, 66, 67, and 68; (e) the LCDR2 comprises an amino acid sequence that is about 80%, 85%, 90%, 95%, 96%, 97%, 98%, 99% to about 100% identical to an amino acid sequence selected from the group consisting of SEQ ID NOs: 80, 71, and 82; and (f) the LCDR3 comprises an amino acid sequence that is about 80%, 85%, 90%, 95%, 96%, 97%, 98%, 99% to about 100% identical to an amino acid sequence as set forth in SEQ ID NO: 90 or 91.

In another aspect, the present invention provides an isolated antigen binding molecule, e.g., an antibody or antigen binding fragment thereof, that binds to human PD-L1. The isolated antigen binding molecule, e.g., the antibody or antigen-binding fragment thereof, includes (a) a heavy chain variable region (HCVR) comprising an amino acid sequence that is about 80%, 85%, 90%, 95%, 96%, 97%, 98%, 99% to about 100% identical to an amino acid sequence selected from the group consisting of SEQ ID NOs: 110-117; and (b) a light chain variable region (LCVR) comprising an amino acid sequence that is about 80%, 85%, 90%, 95%, 96%, 97%, 98%, 99% to about 100% identical to an amino acid sequence selected from the group consisting of SEQ ID NOs: 136-143.

In still another aspect, the present invention provides an isolated antigen binding molecule, e.g., an antibody or antigen binding fragment thereof, that binds to human PD-L1. The isolated antigen binding molecule, e.g., the antibody or antigen-binding fragment thereof, includes a heavy chain variable (V_{H}) domain comprising from N-terminus to C-terminus, three heavy chain complementarity-determining regions (CDRs), HCDR1, HCDR2, and HCDR3; and a light chain variable (V_{L}) domain comprising from N-terminus to C-terminus, three light chain complementarity-determining regions (CDRs), LCDR1, LCDR2, and LCDR3; wherein (a) the HCDR1 comprises an amino acid sequence S-G-Y-D-M-S (SEQ ID NO: 15) or S-G-Y-D-M-C (SEQ ID NO: 16); (b) the HCDR2 comprises an amino acid sequence X38-I-F-T-X39-S-G-S-T-W-Y-A-N-W-A-K-G (SEQ ID NO: 227), wherein X38 is C or S, X39 is G or T; (c) the HCDR3 comprises an amino acid sequence T-X51-D-G-X52-G-S-F-Y-M-N-L (SEQ ID NO: 235); wherein X51 is K or R, and X52 is A or V; (d) the LCDR1 comprises an amino acid sequence Q-A-S-G-T-I-G-S-N-L-A (SEQ ID NO: 63) or Q-A-S-Q-T-I-G-S-N-L-A (SEQ ID NO: 62); (e) the LCDR2 comprises an amino acid sequence K-A-F-T-L-A-S (SEQ ID NO: 76) or K-T-F-T-L-A-S (SEQ ID NO: 77); and (f) the LCDR3 comprises an amino acid sequence Q-Q-G-A-T-R-I-N-I-D-N-A (SEQ ID NO: 86) or Q-Q-G-A-S-R-I-N-I-D-N-A (SEQ ID NO: 87).

In one embodiment, (a) the HCDR2 comprises an amino acid sequence as set forth in SEQ ID NO: 34 or 35; and (b) the HCDR3 comprises an amino acid sequence as set forth in SEQ ID NO: 45 or 46.

In another embodiment, the isolated antigen binding molecule, e.g., the antibody or the antigen binding fragment thereof, of claim 16, includes (a) the HCDR1 comprises an amino acid sequence set forth in SEQ ID NO: 15, the HCDR2 comprising an amino acid sequence set forth in SEQ ID NO: 34, the HCDR3 comprising an amino acid sequence set forth in SEQ ID NO: 45, the LCDR1 comprising an amino acid sequence set forth in SEQ ID NO: 62, the LCDR2 comprising an amino acid sequence set forth in SEQ ID NO: 76, and the LCDR3 comprising an amino acid sequence set forth in SEQ ID NO: 86; or (b) the HCDR1 comprises an amino acid sequence set forth in SEQ ID NO: 16, the HCDR2 comprising an amino acid sequence set forth in SEQ ID NO: 35, the HCDR3 comprising an amino acid sequence set forth in SEQ ID NO: 46, the LCDR1 comprising an amino acid sequence set forth in SEQ ID NO: 63, the LCDR2 comprising an amino acid sequence set forth in SEQ ID NO: 77, and the LCDR3 comprising an amino acid sequence set forth in SEQ ID NO: 87.

In still another embodiment, the isolated antigen binding molecule, e.g., the antibody or the antigen binding fragment thereof, includes (a) a heavy chain variable region (HCVR) comprising an amino acid sequence as set forth in SEQ ID NOs: 106 or 107; and (b) a light chain variable region (LCVR) comprising an amino acid sequence as set forth in SEQ ID NOs: 132 or 133.

In yet another embodiment the isolated antigen binding molecule, e.g., the antibody or the antigen binding fragment thereof, includes (a) the HCVR comprising an amino acid sequence set forth in SEQ ID NO: 106, and the LCVR comprising an amino acid sequence set forth in SEQ ID NO: 132; or (b) the HCVR comprising an amino acid sequence set forth in SEQ ID NO: 107, and the LCVR comprising an amino acid sequence set forth in SEQ ID NO: 133.

In one aspect, the present invention provides an isolated antigen binding molecule, e.g., an antibody or antigen binding fragment thereof, that binds to human PD-L1. The isolated antigen binding molecule, e.g., the antibody or antigen-binding fragment thereof, includes a heavy chain variable (VH) domain comprising from N-terminus to C-terminus, three heavy chain complementarity-determining regions (CDRs), HCDR1, HCDR2, and HCDR3; and a light chain variable (VL) domain comprising from N-terminus to C-terminus, three light chain complementarity-determining regions (CDRs), LCDR1, LCDR2, and LCDR3; wherein (a) the HCDR1 comprises an amino acid sequence that is about 80%, 85%, 90%, 95%, 96%, 97%, 98%, 99% to about 100% identical to an amino acid sequence as set forth in SEQ ID NO: 15 or 16; (b) the HCDR2 comprises an amino acid sequence that is about 80%, 85%, 90%, 95%, 96%, 97%, 98%, 99% to about 100% identical to an amino acid sequence as set forth in SEQ ID NO: 34 or 35; (c) the HCDR3 comprises an amino acid sequence that is about 80%, 85%, 90%, 95%, 96%, 97%, 98%, 99% to about 100% identical to an amino acid sequence as set forth in SEQ ID NO: 45 or 46; (d) the LCDR1 comprises an amino acid sequence that is about 80%, 85%, 90%, 95%, 96%, 97%, 98%, 99% to about 100% identical to an amino acid sequence as set forth in SEQ ID NOs: 62 or 63; (e) the LCDR2 comprises an amino acid sequence that is about 80%, 85%, 90%, 95%, 96%, 97%, 98%, 99% to about 100% identical to an amino acid sequence as set forth in SEQ ID NOs: 76 or 77; and (f) the LCDR3 comprises an amino acid sequence that is about 80%, 85%, 90%, 95%, 96%, 97%, 98%, 99% to about 100% identical to an amino acid sequence as set forth in SEQ ID NO: 86 or 87.

In another aspect, the present invention provides an isolated antigen binding molecule, e.g., an antibody or antigen binding fragment thereof, that binds to human PD-L1. The isolated antigen binding molecule, e.g., the antibody or antigen-binding fragment thereof, includes (a) a heavy chain variable region (HCVR) comprising an amino acid sequence that is about 80%, 85%, 90%, 95%, 96%, 97%, 98%, 99% to about 100% identical to an amino acid sequence selected from the group consisting of SEQ ID NOs: 106 and 107; and (b) a light chain variable region (LCVR) comprising an amino acid sequence that is about 80%, 85%, 90%, 95%, 96%, 97%, 98%, 99% to about 100% identical to an amino acid sequence selected from the group consisting of SEQ ID NOs: 132 and 133.

In one aspect, the present invention provides an isolated antigen binding molecule, e.g., an antibody or antigen binding fragment thereof, that binds to human PD-L1. The isolated antigen binding molecule, e.g., the antibody or antigen-binding fragment thereof, includes a heavy chain variable (V_{H}) domain comprising from N-terminus to C-terminus, three heavy chain complementarity-determining regions (CDRs), HCDR1, HCDR2, and HCDR3; and a light chain variable (V_{L}) domain comprising from N-terminus to C-terminus, three light chain complementarity-determining regions (CDRs), LCDR1, LCDR2, and LCDR3; wherein (a) the HCDR1 comprises an amino acid sequence S-T-Y-A-L-G (SEQ ID NO: 17) or S-T-Y-A-M-G (SEQ ID NO: 18); (b) the HCDR2 comprises an amino acid sequence S-I-S-I-G-G-A-T-Y-X40-A-X41-W-A-K-G (SEQ ID NO: 228), wherein X40 is F or Y, and X41 is S or T; (c) the HCDR3 comprises an amino acid sequence A-R-N-V-D-X53-I-Y-L-X54-A-F-X55-X56 (SEQ ID NO: 236); wherein X53 is I or S, X54 is D or N, X55 is D or H, and X56 is I or T; (d) the LCDR1 comprises an amino acid sequence Q-A-S-Q-N-I-Y-N-N-L-A (SEQ ID NO: 64); (e) the LCDR2 comprises an amino acid sequence X104-X105-S-T-L-A-S (SEQ ID NO: 259), wherein X104 is R or S, and X105 is A or S; and (f) the LCDR3 comprises an amino acid sequence Q-T-Y-Y-L-T-X109-T-X110-N-A (SEQ ID NO: 262), wherein X109 is S or T, and X110 is I or T.

In one embodiment, (a) the HCDR2 comprises an amino acid sequence as set forth in SEQ ID NO: 36 or 37; (b) the HCDR3 comprises an amino acid sequence as set forth in SEQ ID NO: 47 or 48; (c) the LCDR2 comprises an amino acid sequence as set forth in SEQ ID NO: 78 or 79; and (d) the LCDR3 comprises an amino acid sequence as set forth in SEQ ID NO: 88 or 89.

In another embodiment, (a) the HCDR1 comprising an amino acid sequence set forth in SEQ ID NO: 17, the HCDR2 comprising an amino acid sequence set forth in SEQ ID NO: 36, the HCDR3 comprising an amino acid sequence set forth in SEQ ID NO: 47, the LCDR1 comprising an amino acid sequence set forth in SEQ ID NO: 64, the LCDR2 comprising an amino acid sequence set forth in SEQ ID NO: 78, and the LCDR3 comprising an amino acid sequence set forth in SEQ ID NO: 88; or (b) the HCDR1 comprising an amino acid sequence set forth in SEQ ID NO: 18, the HCDR2 comprising an amino acid sequence set forth in SEQ ID NO: 37, the HCDR3 comprising an amino acid sequence set forth in SEQ ID NO: 48, the LCDR1 comprising an amino acid sequence set forth in SEQ ID NO: 64, the LCDR2 comprising an amino acid sequence set forth in SEQ ID NO: 79, and the LCDR3 comprising an amino acid sequence set forth in SEQ ID NO: 89.

In still another embodiment, the isolated antigen binding molecule, e.g., the antibody or the antigen binding fragment thereof, includes (a) a heavy chain variable region (HCVR) comprising an amino acid sequence as set forth in SEQ ID NO: 108 or 109; and (b) a light chain variable region (LCVR) comprising an amino acid sequence as set forth in SEQ ID NO: 134 or 135.

In yet another embodiment, the isolated antigen binding molecule, e.g., the antibody or the antigen binding fragment thereof, includes (a) the HCVR comprising an amino acid sequence set forth in SEQ ID NO: 108, and the LCVR comprising an amino acid sequence set forth in SEQ ID NO: 134; or (b) the HCVR comprising an amino acid sequence set forth in SEQ ID NO: 109, and the LCVR comprising an amino acid sequence set forth in SEQ ID NO: 135.

In one aspect, the present invention provides an isolated antigen binding molecule, e.g., an antibody or antigen binding fragment thereof, that binds to human PD-L1. The isolated antigen binding molecule, e.g., the antibody or antigen-binding fragment thereof, includes a heavy chain variable (VH) domain comprising from N-terminus to C-terminus, three heavy chain complementarity-determining regions (CDRs), HCDR1, HCDR2, and HCDR3; and a light chain variable (VL) domain comprising from N-terminus to C-terminus, three light chain complementarity-determining regions (CDRs), LCDR1, LCDR2, and LCDR3; wherein (a) the HCDR1 comprises an amino acid sequence that is about 80%, 85%, 90%, 95%, 96%, 97%, 98%, 99% to about 100% identical to an amino acid sequence as set forth in SEQ ID NO: 17 or 18; (b) the HCDR2 comprises an amino acid sequence that is about 80%, 85%, 90%, 95%, 96%, 97%, 98%, 99% to about 100% identical to an amino acid sequence as set forth in SEQ ID NO: 36 or 37; (c) the HCDR3 comprises an amino acid sequence that is about 80%, 85%, 90%, 95%, 96%, 97%, 98%, 99% to about 100% identical to an amino acid sequence set forth in SEQ ID NO: 47 or 48; (d) the LCDR1 comprises an amino acid sequence that is about 80%, 85%, 90%, 95%, 96%, 97%, 98%, 99% to about 100% identical to an amino acid sequence as set forth in SEQ ID NO: 64; (e) the LCDR2 comprises an amino acid sequence that is about 80%, 85%, 90%, 95%, 96%, 97%, 98%, 99% to about 100% identical to an amino acid sequence as set forth in SEQ ID NO: 78 or 79; and (f) the LCDR3 comprises an amino acid sequence that is about 80%, 85%, 90%, 95%, 96%, 97%, 98%, 99% to about 100% identical to an amino acid sequence as set forth in SEQ ID NO: 88 or 89.

In another aspect, the present invention provides an isolated antigen binding molecule, e.g., an antibody or antigen binding fragment thereof, that binds to human PD-L1. The isolated antigen binding molecule, e.g., the antibody or antigen-binding fragment thereof, includes (a) a heavy chain variable region (HCVR) comprising an amino acid sequence that is about 80%, 85%, 90%, 95%, 96%, 97%, 98%, 99% to about 100% identical to an amino acid sequence as set forth in SEQ ID NO: 108 or 109; and (b) a light chain variable region (LCVR) comprising an amino acid sequence that is about 80%, 85%, 90%, 95%, 96%, 97%, 98%, 99% to about 100% identical to an amino acid sequence as set forth in SEQ ID NO: 134 or 135.

In still another aspect, the present invention provides an isolated antigen binding molecule, e.g., an antibody or antigen binding fragment thereof, that binds to human PD-L1. The isolated antigen binding molecule, e.g., the antibody or antigen-binding fragment thereof, includes a heavy chain variable (V_{H}) domain comprising from N-terminus to C-terminus, three heavy chain complementarity-determining regions (CDRs), HCDR1, HCDR2, and HCDR3; and a light chain variable (V_{L}) domain comprising from N-terminus to C-terminus, three light chain complementarity-determining regions (CDRs), LCDR1, LCDR2, and LCDR3; wherein (a) (a) the HCDR1 comprises an amino acid sequence S-S-Y-Y-M-S (SEQ ID NO: 13); (b) the HCDR2 comprises an amino acid sequence C-I-S-G-G-V-T-D-N-A-Y-Y-A-S-W-A-K-G (SEQ ID NO: 32); (c) the HCDR3 comprises an amino acid sequence D-S-S-S-G-Y-F-F-L-L (SEQ ID NO: 43); (d) the LCDR1 comprises an amino acid sequence Q-A-S-Q-N-I-Y-S-N-L-A (SEQ ID NO: 60); (e) the LCDR2 comprises an amino acid sequence G-A-S-N-L-R-S (SEQ ID NO: 74); and (f) the LCDR3 comprises an amino acid sequence Q-E-G-Y-S-I-G-N-V-D-N-P (SEQ ID NO: 84).

In one embodiment, the isolated antigen binding molecule, e.g., the antibody or the antigen binding fragment thereof, includes the HCVR comprising an amino acid sequence set forth in SEQ ID NO: 104, and the LCVR comprising an amino acid sequence set forth in SEQ ID NO: 130.

In one aspect, the present invention provides an isolated antigen binding molecule, e.g., an antibody or antigen binding fragment thereof, that binds to human PD-L1. The isolated antigen binding molecule, e.g., the antibody or antigen-binding fragment thereof, includes a heavy chain variable (VH) domain comprising from N-terminus to C-terminus, three heavy chain complementarity-determining regions (CDRs), HCDR1, HCDR2, and HCDR3; and a light chain variable (VL) domain comprising from N-terminus to C-terminus, three light chain complementarity-determining regions (CDRs), LCDR1, LCDR2, and LCDR3; wherein (a) the HCDR1 comprises an amino acid sequence that is about 80%, 85%, 90%, 95%, 96%, 97%, 98%, 99% to about 100% identical to an amino acid sequence as set forth in SEQ ID NO: 13; (b) the HCDR2 comprises an amino acid sequence that is about 80%, 85%, 90%, 95%, 96%, 97%, 98%, 99% to about 100% identical to an amino acid sequence as set forth in SEQ ID NO: 32; (c) the HCDR3 comprises an amino acid sequence that is about 80%, 85%, 90%, 95%, 96%, 97%, 98%, 99% to about 100% identical to an amino acid sequence set forth in SEQ ID NO: 43; (d) the LCDR1 comprises an amino acid sequence that is about 80%, 85%, 90%, 95%, 96%, 97%, 98%, 99% to about 100% identical to an amino acid sequence as set forth in SEQ ID NO: 60; (e) the LCDR2 comprises an amino acid sequence that is about 80%, 85%, 90%, 95%, 96%, 97%, 98%, 99% to about 100% identical to an amino acid sequence as set forth in SEQ ID NO: 74; and (f) the LCDR3 comprises an amino acid sequence that is about 80%, 85%, 90%, 95%, 96%, 97%, 98%, 99% to about 100% identical to an amino acid sequence as set forth in SEQ ID NO: 84.

In another aspect, the present invention provides an isolated antigen binding molecule, e.g., an antibody or antigen binding fragment thereof, that binds to human PD-L1. The isolated antigen binding molecule, e.g., the antibody or antigen-binding fragment thereof, includes (a) a heavy chain variable region (HCVR) comprising an amino acid sequence that is about 80%, 85%, 90%, 95%, 96%, 97%, 98%, 99% to about 100% identical to an amino acid sequence as set forth in SEQ ID NO: 104; and (b) a light chain variable region (LCVR) comprising an amino acid sequence that is about 80%, 85%, 90%, 95%, 96%, 97%, 98%, 99% to about 100% identical to an amino acid sequence as set forth in SEQ ID NO: 130.

In one aspect, the present invention provides an isolated antigen binding molecule, e.g., an antibody or antigen binding fragment thereof, that binds to human PD-L1. The isolated antigen binding molecule, e.g., the antibody or antigen-binding fragment thereof, includes a heavy chain variable (V_{H}) domain comprising from N-terminus to C-terminus, three heavy chain complementarity-determining regions (CDRs), HCDR1, HCDR2, and HCDR3; and a light chain variable (V_{L}) domain comprising from N-terminus to C-terminus, three light chain complementarity-determining regions (CDRs), LCDR1, LCDR2, and LCDR3; wherein (a) the HCDR1 comprises an amino acid sequence S-S-Y-Y-M-I (SEQ ID NO: 14); (b) the HCDR2 comprises an amino acid sequence Y-V-Y-T-G-S-G-N-T-W-Y-A-S-W-A-K-G (SEQ ID NO: 33); (c) the HCDR3 comprises an amino acid sequence A-S-G-A-D-G-V-Y-D-W-G-W-D-I (SEQ ID NO: 44); (d) the LCDR1 comprises an amino acid sequence Q-A-S-Q-S-I-Y-S-L-L-A (SEQ ID NO: 61); (e) the LCDR2 comprises an amino acid sequence G-A-S-N-L-E-S (SEQ ID NO: 75); and (f) the LCDR3 comprises an amino acid sequence Q-N-N-Y-D-S-G-R-I-Y-G-L-A (SEQ ID NO: 85).

In one embodiment, the isolated antigen binding molecule, e.g., the antibody the antigen binding fragment thereof, includes the HCVR comprising an amino acid sequence set forth in SEQ ID NO: 105, and the LCVR comprising an amino acid sequence set forth in SEQ ID NO: 131.

In another aspect, the present invention provides an isolated antigen binding molecule, e.g., an antibody or antigen binding fragment thereof, that binds to human PD-L1. The isolated antigen binding molecule, e.g., the antibody or antigen-binding fragment thereof, includes a heavy chain variable (VH) domain comprising from N-terminus to C-terminus, three heavy chain complementarity-determining regions (CDRs), HCDR1, HCDR2, and HCDR3; and a light chain variable (VL) domain comprising from N-terminus to C-terminus, three light chain complementarity-determining regions (CDRs), LCDR1, LCDR2, and LCDR3; wherein (a) the HCDR1 comprises an amino acid sequence that is about 80%, 85%, 90%, 95%, 96%, 97%, 98%, 99% to about 100% identical to an amino acid sequence as set forth in SEQ ID NO: 14; (b) the HCDR2 comprises an amino acid sequence that is about 80%, 85%, 90%, 95%, 96%, 97%, 98%, 99% to about 100% identical to an amino acid sequence as set forth in SEQ ID NO: 33; (c) the HCDR3 comprises an amino acid sequence that is about 80%, 85%, 90%, 95%, 96%, 97%, 98%, 99% to about 100% identical to an amino acid sequence set forth in SEQ ID NO: 44; (d) the LCDR1 comprises an amino acid sequence that is about 80%, 85%, 90%, 95%, 96%, 97%, 98%, 99% to about 100% identical to an amino acid sequence as set forth in SEQ ID NO: 61; (e) the LCDR2 comprises an amino acid sequence that is about 80%, 85%, 90%, 95%, 96%, 97%, 98%, 99% to about 100% identical to an amino acid sequence as set forth in SEQ ID NO: 75; and (f) the LCDR3 comprises an amino acid sequence that is about 80%, 85%, 90%, 95%, 96%, 97%, 98%, 99% to about 100% identical to an amino acid sequence as set forth in SEQ ID NO: 85.

In still another aspect, the present invention provides an isolated antigen binding molecule, e.g., an antibody or antigen binding fragment thereof, that binds to human PD-L1. The isolated antigen binding molecule, e.g., the antibody or antigen-binding fragment thereof, includes (a) a heavy chain variable region (HCVR) comprising an amino acid sequence that is about 80%, 85%, 90%, 95%, 96%, 97%, 98%, 99% to about 100% identical to an amino acid as set forth in SEQ ID NO: 105; and (b) a light chain variable region (LCVR) comprising an amino acid sequence that is about 80%, 85%, 90%, 95%, 96%, 97%, 98%, 99% to about 100% identical to an amino acid sequence as set forth in SEQ ID NO: 131.

In one embodiment, the binding of the antibody to an PD-L1, or a cell surface PD-L1, is determined using flow cytometry-based assays or ELISA-based assays as described in Examples 3, 4, and 5, or substantial similar assays thereof. In another embodiment, the competition for binding to an PD-L1 or a cell surface PD-L1 by the antibody is determined using an assay known in the art such as the assay described in Harms, et al., Microtiter plate-based antibody-competition assay to determine binding affinities and plasma/blood stability of CXCR4 ligands, Scientific Reports, 2020:10:16036, doi.org/10.1038/s41598-020-73012-4, or substantial similar assay thereof. In still another embodiment, the blocking of the interaction between PD-1 and PD-L1 is determined using an assay as described in Example 5, or substantially similar assay thereof. In another embodiment, the enhancement of an immune response is determined using methods well known in the art, such as the increase of concentration of inflammatory cytokines in a tissue, increase in the number of cytotoxic CD8+ T cells.

In one embodiment, the antibody blocks the PD-1/PD-L1 interaction by at least about 10%, about 20%, about 30%, about 40%, about 50%, about 60%, about 70%, about 80%, about 90%, about 95%, or about 100%. In another embodiment, the antibody enhances an immune response by at least about 10%, about 20%, about 30%, about 40%, about 50%, about 60%, about 70%, about 80%, about 90%, about 95%, about 100%, about 1.5-fold, about 2-folds, about 4-folds, or more.

In still another embodiment, the antibody specifically binds to human PD-L1 and/or cynomolgus PD-L1. In yet another embodiment, the antibody specifically binds to human PD-L1 and/or cynomolgus PD-L1 with similar affinity. In one embodiment, the antibody does not bind to non-primate PD-L1 or binds to non-primate PD-L1 with an affinity that is significantly lower than that of human PD-L1 and/or cynomolgus PD-L1. In various aspects of the invention and embodiments thereof, the cynomolgus PD-L1 comprises a sequence as set forth in

In one aspect, the present invention provides an isolated antigen binding molecule, e.g., an antibody, that competes for binding to human PD-L1 with an antibody of any aspect.

In one embodiment, the antigen binding molecule, e.g., the antibody, is a humanized antibody or a chimeric antibody. In another embodiment, the antibody comprises a heavy chain constant region of a class selected from IgA, IgD, IgE, IgG, or IgM. In still another embodiment, the antibody comprises a heavy chain constant region of the class IgG, and wherein the IgG is selected from the group consisting of IgG1, IgG2, IgG3, and IgG4.

In another aspect, the present invention provides an isolated polynucleotide encoding the antigen binding molecule, e.g., the antibody of any aspects and the various embodiments thereof, an HCVR thereof, an LCVR thereof, a light chain thereof, a heavy chain thereof, or an antigen binding fragment thereof.

In still another aspect, the present invention provides an expression vector that includes comprising the polynucleotide.

In yet another aspect, the present invention provides a recombinant cell that includes the polynucleotide or the expression vector.

In one aspect, the present invention provides a method of producing the antigen binding molecule, e.g., the antibody of any aspects and the various embodiments thereof. The method includes expressing the antibody in the recombinant cell and isolating the expressed antibody.

In one aspect, the present invention provides a pharmaceutical composition. The pharmaceutical composition includes the antigen binding molecule, e.g., the antibody, of any aspects and various embodiments thereof, and a pharmaceutically acceptable carrier or diluent.

In one embodiment, the antibody in the pharmaceutical composition is in an amount effective to (a) specifically bind to a cell surface human or cynomolgus PD-L1; (b) blocks the interaction between PD-1 and PD-L1; (c) induces ADCC; (d) induces internalization of PD-L1; e) improve the immune response of an immune cell; (f) elicits cytotoxicity of PD-L1 expressing cells via the conjugation with cytotoxic agents; and (g) any combination of (a)-(f).

In one aspect, the present invention provides a method of blocking the binding of a PD-1 to a PD-L1 expressed on a cell surface, comprising contacting the cell with the isolated antibody of any aspect or the pharmaceutical composition of any aspect, thereby blocking the binding of PD-1 to PD-L1. In one embodiment, the binding of PD-1 to PD-L1 is reduced by at least about 10%, about 20%, about 30%, about 40%, about 50%, about 60%, about 70%, about 80%, about 90%, about 95%, or about 100%. In another embodiment, the method is used in treating cancer.

In another aspect, the present invention provides a method of enhancing an immune response in a subject. The method includes administering an isolated antibody of any aspect or the pharmaceutical composition of any aspect to the subject, thereby enhancing the immune response in the subject. In one embodiment, the immune response includes, but is not limited to a) reversing T cell inactivation and function; b) promoting T cell proliferation; c) enhancing NK cell function; d) enhance the T-cell-mediated anti-tumor immune response; or e) reducing the immunosuppression in a tumor microenvironment. In certain embodiments, the methods of the invention increase an immune response by at least about 10%, about 20%, about 50%, about 60%, about 70%, about 80%, about 90%, about 1-fold, about 2-folds, about 4 folds, or more, as compared to a baseline level.

In still another aspect, the present invention provides a method of inhibiting growth of a tumor in a subject. The method includes administering an isolated antibody of any aspect or the pharmaceutical composition of any aspect to the subject, thereby inhibiting growth of the tumor.

In yet another aspect, the present invention provides a method of treating cancer in a subject, comprising administering an isolated antibody of any aspect or the pharmaceutical composition of aspect, thereby treating the cancer. In one embodiment, the cancer is any cancer described herein. In one particular embodiment, the cancer is selected from the group of non-small cell lung cancer (NSCLC), small cell lung cancer (SCLC), head and neck squamous cell carcinoma (HNSCC), bladder cancer (BC), esophageal squamous cell carcinoma (ESCC), triple negative breast cancer (TNBC), renal cell carcinoma (RCC), colorectal cancer (CRC), hepatocellular carcinoma (HCC), large B cell lymphoma (BCL), Merkel cell carcinoma (MCC), cervical cancer (CC), classical Hodgkin's lymphoma (cHL), and microsatellite instability high (MSI-H), and mismatch repair-deficient (dMMR).

In one embodiment, the method of any of above aspect results in activating T cells and directing them to kill a tumor target cell.

In another embodiment, the method of any of above aspect further includes administering an additional therapeutic agent. In one embodiment, the additional therapeutic agent includes any therapeutic agent described herein. In another embodiment, the additional therapeutic agent comprises an anti-tumor agent, radiotherapy, a chemotherapeutic agent, a surgery, a cancer vaccine, an agonist to a stimulatory receptor of an immune cell, a cytokine, a cell therapy, or a checkpoint inhibitor. In one embodiment, the additional therapeutic agent is an antibody, including multi-specific antibody, e.g., bispecific antibody.

In still another embodiment, checkpoint inhibitor is an agent that inhibits TIGIT, CTLA-4, PD-L2, LAG-3, TIM-3, neuritin, BTLA, CECAM-1, CECAM-5, IL-1R8, VISTA, LAIR1, LILRB1, LILRB2, LILRB3, LILRB4, LILRB5, CD96, CD112R, CD 160, 2B4, TGFβ-R, KIR, NKG2A, MICA, MICB, A2aR, A2bR, and any combination thereof. In one embodiment, the CTLA inhibitor is ipilimumab, cadonilimab, YH001 (Encure Biopharma).

In yet another embodiment, the additional therapeutic agent is an agonist to a stimulatory receptor of an immune cell selected from OX40, CD2, CD16, CD27, CDS, ICAM-1, LFA-1, ICOS (CD278), 4-1 BB (CD137), GITR, CD28, CD30, CD40, BAFFR, HVEM, CD7, LIGHT, NKG2C, NKG2D, SLAMF7, NKp46, NKp80, CD160, B7-H3, CD83 ligand, and any combination thereof.

In one embodiment, the additional therapeutic agent is formulated in the same pharmaceutical composition as the antibody. In another embodiment, the additional therapeutic agent is formulated in a different pharmaceutical composition from the antibody.

In still another embodiment, the additional therapeutic agent is administered prior to the antigen binding molecule, e.g., antibody, of various aspect. In yet another embodiment, the additional therapeutic agent is administered subsequent to the antigen biding molecule, e.g., antibody, subsequently to administering the antibody. In another embodiment, the additional therapeutic agent is administered concurrently with the antigen binding molecule, e.g., the antibody.

In one aspect, the present invention provides a method of inducing antibody-dependent cellular cytotoxicity (ADCC) of a PD-L1 expressing cell, comprising contacting the cell with the isolated antibody of any aspect or the pharmaceutical composition of any aspect, thereby inducing the antibody-dependent cellular cytotoxicity (ADCC) of the PD-L1 expressing cell.

In another aspect, the present invention provides a method of killing a PD-L1 expressing cell, comprising contacting the cell with the isolated antibody of any aspect or the pharmaceutical composition of any aspect, wherein the isolated antibody is conjugated to a cytotoxic agent, thereby killing the PD-L1 expressing cell.

In one aspect, the present invention provides a kit. The kit includes the pharmaceutical composition of any aspect. In one embodiment, the pharmaceutical composition further comprises any one or more of the additional therapeutic agents described herein.

### BRIEF DESCRIPTION OF THE FIGURES

FIG. 1 shows that exemplary antibodies of the present disclosure effectively block the interaction between PD-1 and PD-L1.

FIGS. 2A, 2B and 2C show that the exemplary antibodies of the present disclosure induce internalization upon binding to cell surface PD-L1.

FIG. 3 shows that exemplary antibodies of the present disclosure conjugated with a cytotoxic agent elicit potent killing of PD-L1 expressing cells.

FIGS. 4A and 4B show epitope binning of exemplary antibodies of the present disclosure with Avelumab, as well as certain other anti-PD-L1 antibodies with Avelumab.

### DETAILED DESCRIPTION

The invention and accompanying drawings will now be discussed to enable one skilled in the art to practice the present invention. The skilled artisan will understand, however, that the inventions described below can be practiced without employing these specific details, or that they can be used for purposes other than those described herein. Indeed, they can be modified and can be used in conjunction with products and techniques known to those of skill in the art considering the present disclosure. The drawings and descriptions are intended to be exemplary of various aspects of the invention and are not intended to narrow the scope of the appended claims. Furthermore, it will be appreciated that the drawings may show aspects of the invention in isolation and the elements in one figure may be used in conjunction with elements shown in other figures.

It will be appreciated that reference throughout this specification to aspects, features, advantages, or similar language does not imply that all the aspects and advantages may be realized with the present invention should be or are in any single embodiment of the invention. Rather, language referring to the aspects and advantages is understood to mean that a specific aspect, feature, advantage, or characteristic described in connection with an embodiment is included in at least one embodiment of the present invention. Thus, discussion of the aspects and advantages, and similar language, throughout this specification may, but does not necessarily, refer to the same embodiment.

The described aspects, features, advantages, and characteristics of the invention may be combined in any suitable manner in one or more further embodiments. Furthermore, one skilled in the relevant art will recognize that the invention may be practiced without one or more of the specific aspects or advantages of a particular embodiment. In other instances, additional aspects, features, and advantages may be recognized and claimed in certain embodiments that may not be present in all embodiments of the invention.

Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present invention. The described aspects and embodiments of the application are not limited to the methods and materials described.

Further, with respect to the teachings in the present invention, any cited references, any issued patent or patent application publication described in this application is expressly incorporated by reference herein.

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The use of the terms “a” and “an” and “the” and similar referents in the context of describing the invention (especially in the context of the following claims) are to be construed to cover both the singular and the plural (i.e., one or more), unless otherwise indicated herein or clearly contradicted by context. The terms “comprising, “having,” “including,” and “containing” are to be construed as open-ended terms (i.e., meaning “including, but not limited to”) unless otherwise noted. Recitation of ranges of values herein are merely intended to serve as a shorthand method of referring individually to each separate value recited or falling within the range, unless otherwise indicated herein, and each separate value is incorporated into the specification as if it were individually recited.

Where the phrases “in one embodiment”, “in another embodiment” “in other embodiments”, “in some embodiments” or “in certain embodiments” are used, the present disclosure should be construed as embracing combinations of any of the features defining the different embodiments described therein, unless the features are not combinable with one another, are mutually exclusive, or are expressly disclaimed herein.

The term “about” or “approximately” usually means within 10%, preferably within 5%, or more preferably within 1%, of a given value or range.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about”, it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

As used herein, the term “agent” is used with reference to any substance, compound (e.g., molecule), supramolecular complex, material, or combination or mixture thereof. A compound may be any agent that can be represented by a chemical formula, chemical structure, or sequence. Example of agents, include, e.g., small molecules, polypeptides, nucleic acids (e.g., RNAi agents, antisense oligonucleotide, aptamers), lipids, polysaccharides, etc. In general, agents may be obtained using any suitable method known in the art. The ordinary skilled artisan will select an appropriate method based, e.g., on the nature of the agent. An agent may be at least partly purified. In some embodiments an agent may be provided as part of a composition, which may contain, e.g., a counter-ion, aqueous or non-aqueous diluent or carrier, buffer, preservative, or other ingredient, in addition to the agent, in various embodiments. In some embodiments an agent may be provided as a salt, ester, hydrate, or solvate. In some embodiments an agent is cell-permeable, e.g., within the range of typical agents that are taken up by cells and acts intracellularly, e.g., within mammalian cells, to produce a biological effect. Certain compounds may exist in particular geometric or stereoisomeric forms. Such compounds, including cis- and trans-isomers, E- and Z-isomers, R- and S-enantiomers, diastereomers, (D)-isomers, (L)-isomers, (−)- and (+)-isomers, racemic mixtures thereof, and other mixtures thereof are encompassed by this disclosure in various embodiments unless otherwise indicated. Certain compounds may exist in a variety or protonation states, may have a variety of configurations, may exist as solvates (e.g., with water (i.e., hydrates) or common solvents) and/or may have different crystalline forms (e.g., polymorphs) or different tautomeric forms. Embodiments exhibiting such alternative protonation states, configurations, solvates, and forms are encompassed by the present disclosure where applicable.

In certain embodiment and depending on the context, an “agent” also includes a method of treatment, such as radiotherapy, chemotherapy, or surgery.

The term “agonist” refers to a substance which promotes (i.e., induces, causes, enhances, or increases) the biological activity or effect of another molecule. The term agonist encompasses substances which bind receptor, such as an antibody, and substances which promote receptor function without binding thereto (e.g., by activating an associated protein).

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gin; Q), Glycine (Gly; G); histidine (His; H), isoleucine (He; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The term “antagonist” or “inhibitor” refers to a substance that prevents, blocks, inhibits, neutralizes, or reduces a biological activity or effect of another molecule, such as a receptor.

The term “antibody”, as used herein, means any antigen binding molecule or molecular complex comprising at least one complementarity determining region (CDR) that specifically binds to or interacts with a particular antigen (e.g., PD-L1). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}I). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-PD-L1 antibody (or antigen binding fragment thereof) may be identical to the murine or human germ line sequences or may be naturally or artificially modified. An amino acid consensus sequence may be defined based on a side-by-side analysis of two or more CDRs.

The term “antibody”, as used herein, also includes antigen binding fragments of full antibody molecules. The terms “antigen binding portion” of an antibody, “antigen binding fragment” of an antibody, and the like, as used herein, include any naturally occurring, enzymatically obtainable, synthetic, or genetically engineered polypeptide or glycoprotein that specifically binds an antigen to form a complex. Antigen binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains.

Non-limiting examples of antigen binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen binding fragment,” as used herein.

When describing polypeptide domain arrangements with hyphens between individual domains (e.g., CH2-CH3), it should be understood that the order of the listed domains is from the N-terminus to the C-terminus.

An antigen binding fragment of an antibody will typically comprise at least one variable domain. The variable domain may be of any size or amino acid composition and will generally comprise at least one CDR which is adjacent to or in frame with one or more framework sequences. In antigen binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen binding fragment of an antibody may contain a monomeric V_{H} or V_{L} domain.

In certain embodiments, an antigen binding fragment of an antibody may contain at least one variable domain covalently linked to at least one constant domain. Non-limiting, exemplary configurations of variable and constant domains that may be found within an antigen binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) VL-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen binding fragment may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen binding fragments may be monospecific or multispecific (e.g., bispecific). A multispecific antigen binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen binding fragment of an antibody of the present invention using routine techniques available in the art.

The antibodies of the invention may be isolated antibodies. An “isolated” molecule, such as an isolated antibody or an isolated polypeptide, as used herein, means a molecule, e.g., an antibody, that has been identified and separated and/or recovered from at least one component of its natural environment. For example, a molecule, e.g., an antibody, that has been separated or removed from at least one component of an organism, or from a tissue or cell in which the antibody naturally exists or is naturally produced, is an “isolated” molecule, e.g., antibody, for purposes of the present invention. An isolated molecule, e.g., an antibody also includes a molecule, e.g., an antibody in situ within a recombinant cell. In certain embodiments, isolated molecules, e.g., antibodies, are molecules, e.g., antibodies that have been subjected to at least one purification or isolation step. According to certain embodiments, an isolated molecule, e.g., antibody may be substantially free of other cellular material and/or chemicals.

The present invention also includes one-arm antibodies that bind PD-L1. As used herein, a “one-arm antibody” means an antigen binding molecule comprising a single antibody heavy chain and a single antibody light chain. The one-arm antibodies of the present invention may comprise any of the HCVR/LCVR or CDR amino acid sequences as set forth in Tables 1-20.

The anti-PD-L1 antibodies herein, or the antigen binding fragments thereof, may comprise one or more amino acid substitutions, insertions and/or deletions in the framework and/or CDR regions of the heavy and light chain variable domains as compared to the corresponding germline sequences from which the antigen binding molecules, e.g., antibodies or antigen binding fragments were derived. Such mutations can be readily ascertained by comparing the amino acid sequences disclosed herein to germline sequences available from, for example, public antibody sequence databases. The present invention includes antibodies, and the antigen binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can produce numerous antibodies and antigen binding fragments, which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the frameworks and/or CDR residue(s) are mutated to the corresponding residue(s) of a different germline sequence (i.e., a germline sequence that is different from the germline sequence from which the antibody was originally derived). Furthermore, the antibodies, or the antigen binding domains thereof, of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies, or the antigen binding fragments thereof, that contain one or more germline mutations can be easily tested for one or more desired property such as, improved binding specificity, increased binding affinity, improved or enhanced antagonistic or agonistic biological properties (as the case may be), reduced immunogenicity, etc. Antibodies, or the antigen binding fragments thereof, obtained in this general manner are encompassed within the present invention.

The present invention also includes anti-PD-L1 antibodies comprising variants of any of the HCVR, LCVR, and/or CDR amino acid sequences disclosed herein. Exemplary variants included within this aspect of the invention include variants of any of the HCVR, LCVR, and/or CDR amino acid sequences disclosed herein having one or more conservative substitutions. For example, the present invention includes anti-PD-L1 antibodies and antigen binding proteins having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences set forth in the Tables herein.

Light chains are classified as either kappa or lambda (K, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

As used herein, the term “light chain constant region” or “C_{L}” are used interchangeably herein with reference to amino acid sequences derived from an antibody light chain. Preferably, the light chain constant region comprises at least one of a constant kappa domain or constant lambda domain.

As used herein, the term “heavy chain constant region” includes amino acid sequences derived from an immunoglobulin heavy chain. A polypeptide comprising a heavy chain constant region comprises at least one of: a C_{H}1 domain, a hinge (e.g., upper, middle, and/or lower hinge region) domain, a C_{H}2 domain, a C_{H}3 domain, or a variant or fragment thereof. For example, an antigen binding polypeptide for use in the disclosure may comprise a polypeptide chain comprising a C_{H}1 domain; a polypeptide chain comprising a C_{H}1 domain, at least a portion of a hinge domain, and a C_{H}2 domain; a polypeptide chain comprising a C_{H}1 domain and a C_{H}3 domain; a polypeptide chain comprising a C_{H}1 domain, at least a portion of a hinge domain, and a C_{H}3 domain, or a polypeptide chain comprising a C_{H}1 domain, at least a portion of a hinge domain, a C_{H}2 domain, and a C_{H}3 domain. In some embodiments, a polypeptide of the disclosure comprises a polypeptide chain comprising a C_{H}3 domain. Further, an antibody for use in the disclosure may lack at least a portion of a C_{H}2 domain (e.g., all or part of a C_{H}2 domain). It should be understood that the heavy chain constant region may be modified such that they vary in amino acid sequence from the naturally occurring immunoglobulin molecule.

The heavy chain constant region of an antibody disclosed herein may be derived from different immunoglobulin molecules. For example, a heavy chain constant region of a polypeptide may comprise a C_{H}1 domain derived from an IgG₁ molecule and a hinge region derived from an IgG₃ molecule. In another example, a heavy chain constant region can comprise a hinge region derived, in part, from an IgG₁ molecule and, in part, from an IgG₃ molecule. In another example, a heavy chain portion can comprise a chimeric hinge derived, in part, from an IgG₁ molecule and, in part, from an IgG₄ molecule.

The subunit structures and three-dimensional configurations of the constant regions of the various immunoglobulin classes are well known. As used herein, the term “V_{H} domain” includes the N terminal variable domain of an immunoglobulin heavy chain and the term “C_{H}1 domain” includes the first (most N terminal) constant region domain of an immunoglobulin heavy chain. The C_{H}1 domain is adjacent to the V_{H} domain and is N-terminal to the hinge region of an immunoglobulin heavy chain molecule.

As used herein the term “C_{H}2 domain” includes the portion of a heavy chain molecule that extends, e.g., from about residue 244 to residue 360 of an antibody using conventional numbering schemes (residues 244 to 360, Kabat numbering system; and residues 231-340, EU numbering system). The C_{H}2 domain is unique in that it is not closely paired with another domain. Rather, two N-linked branched carbohydrate chains are interposed between the two C_{H}2 domains of an intact native IgG molecule. The C_{H}3 domain extends from the C_{H}2 domain to the C-terminal of the IgG molecule and comprises approximately 108 residues.

As used herein, the term “hinge region” includes the portion of a heavy chain molecule that joins the C_{H}1 domain to the C_{H}2 domain. This hinge region comprises approximately 25 residues and is flexible, thus allowing the two N-terminal antigen binding regions to move independently. Hinge regions can be subdivided into three distinct domains: upper, middle, and lower hinge domains.

As used herein the term “disulfide bond” includes a covalent bond formed between two sulfur atoms. The amino acid cysteine comprises a thiol group that can form a disulfide bond or bridge with a second thiol group. In most naturally occurring IgG molecules, the C_{H}1 and C_{L} regions are linked by a disulfide bond and the two heavy chains are linked by two disulfide bonds at positions corresponding to 239 and 242 using the Kabat numbering system (position 226 or 229, EU numbering system).

The term “antibody” encompasses various broad classes of polypeptides that can be distinguished biochemically. Those skilled in the art will appreciate that heavy chains are classified as alpha, delta, epsilon, gamma, and mu, or α, δ, ε, γ and μ) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG1, IgG2, IgG3, IgG4, IgG5, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are within the scope of the present disclosure, the following discussion will generally be directed to the IgG class of immunoglobulin molecules.

Antibodies of the disclosure include, but are not limited to, polyclonal, monoclonal, multispecific, bispecific, trispecific, human, humanized, primatized, chimeric and single chain antibodies. Antibodies disclosed herein may be from any animal origin, including birds and mammals. Preferably, the antibodies are human, murine, donkey, rabbit, goat, guinea pig, camel, llama, horse, or chicken antibodies. In some embodiments, the variable region may be condricthoid in origin (e.g., from sharks).

As used herein, the phrase “chimeric antibody,” refers to an antibody where the immunoreactive region or site is obtained or derived from a first species and the constant region (which may be intact, partial or modified in accordance with the instant disclosure) is obtained from a second species. In certain embodiments the target binding region or site will be from a non-human source (e.g., mouse or primate) and the constant region is human.

The term “humanized antibody” as used herein, refers to a genetically engineered non-human antibody, which contains human antibody constant domains and non-human variable domains modified to contain a high level of sequence homology to human variable domains. This can be achieved by grafting the six non-human antibody complementarity-determining regions (CDRs), which together form the antigen binding site, onto a homologous human acceptor framework region (FR). In order to reconstitute the binding affinity and specificity of the parental antibody, the substitution of framework residues from the parental antibody (i.e., the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

A “single-chain fragment variable” or “scFv” refers to a fusion protein of the variable regions of the heavy (VH) and light chains (VL) of immunoglobulins. In some aspects, the regions are connected with a short linker peptide of ten to about 25 amino acids. The linker can be rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker.

The antibodies of the invention may, in some embodiments, be recombinant antibodies. The term “recombinant antibody”, as used herein, is intended to include all antibodies that are prepared, expressed, created or isolated by recombinant means, such as antibodies expressed using a recombinant expression vector transfected into a host cell, antibodies isolated from a recombinant, combinatorial antibody library or antibodies prepared, expressed, created or isolated by any other means that involves splicing of immunoglobulin gene sequences to other DNA sequences. Such recombinant antibodies have variable and constant regions derived from germline immunoglobulin sequences. In certain embodiments, however, such recombinant antibodies are subjected to in vitro mutagenesis and thus the amino acid sequences of the VH and VL regions of the recombinant antibodies are sequences that, while derived from and related to germ line VH and VL sequences, may not naturally exist within the antibody germ line repertoire in vivo.

The term “conjugate” or “antibody conjugate” refers to an antibody linked to one or more agents. The antibody can be covalently linked to the agent via a covalent bond or a linker. In certain embodiments, the linker is covalently bonded to the antibody and also covalently bonded to the agent. In certain embodiments, the linker is linked to the antibody and/or the agent via non-covalent means. In certain embodiments, the linker is linked to the agent via a covalent bond and linked to the antibody via specifical binding. In certain embodiments, the linker is a moiety that can specifically binds to the antibody, e.g., an antibody that binds to the Fc region of the antibody.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstances, an epitope may include moieties of saccharides, phosphoryl groups, or sulfonyl groups on the antigen.

The term “immunoconjugate” refers to an antibody which is fused by covalent linkage to a peptide or small molecule drug. The peptide or small molecule drug can be linked to the C-terminus of a constant heavy chain or to the N-terminus of a variable light and/or heavy chain.

The terms, “improve,” “increase,” or “reduce,” as used in this context, indicate values or parameters relative to a baseline/control/reference measurement, such as a measurement in a cell or a tissue prior to initiation of the treatment described herein, or a measurement in a cell or a tissue in the absence of the treatment described herein, a measurement in the same individual prior to initiation of the treatment described herein, or a measurement in a control individual (or a standard measurement derived from multiple control individuals, such as the average value of the multiple control individuals) in the absence of the treatment described herein. A “control individual” is an individual with similar condition, e.g., an individual afflicted with the same cell proliferative disorder as the individual being treated, who is about the same age as the individual being treated (to ensure that the stages of the disease in the treated individual and the control individual(s) are comparable). The individual (also referred to as “patient” or “subject”) being treated may be a fetus, infant, child, adolescent, or adult human with a cell proliferative disorder.

As used herein, the term “recombinant,” as it refers to polypeptide or nucleic acid, refers to polypeptides or polynucleotides that do not exist naturally and which may be created by combining polynucleotides or polypeptides in arrangements that would not normally occur together. The term “recombinant cell” is defined herein as a non-naturally occurring host cell comprising one or more (e.g., two, several) heterologous polynucleotides.

The term “prevent” refers to a decrease in the occurrence of disease symptoms (e.g., associated with PD-L1 activity or function thereof) in a patient. As indicated above, the prevention may be complete (no detectable symptoms) or partial, such that fewer symptoms are observed than would likely occur absent treatment.

The phrase “small molecule drug” refers to a molecular entity, often organic or organometallic, that is not a polymer, that has medicinal activity, and that has a molecular weight less than about 2 kDa, less than about 1 kDa, less than about 900 Da, less than about 800 Da or less than about 700 Da. The term encompasses most medicinal compounds termed “drugs” other than protein or nucleic acids, although a small peptide or nucleic acid analog can be considered a small molecule drug. Examples include chemotherapeutic anticancer drugs and enzymatic inhibitors. Small molecules drugs can be derived synthetically, semi-synthetically (i.e., from naturally occurring precursors), or biologically.

As used herein, the terms “specific binding” or “specifically binds” refer to an ability to discriminate between possible binding partners in the environment in which binding is to occur. In some embodiments, an antibody that interacts, e.g., preferentially interacts, with one particular antigen when other potential antibodies are present is said to “bind specifically” to the antigen with which it interacts. In some embodiments, specific binding is assessed by detecting or determining the degree of association between the antibody and its targeted antigen; in some embodiments, specific binding is assessed by detecting or determining degree of dissociation of an antibody-antigen complex. In some embodiments, specific binding is assessed by detecting or determining ability of the antibody to compete with an alternative interaction between its target and another antibody. In some embodiments, specific binding is assessed by performing such detections or determinations across a range of concentrations. In general, an antibody binds to an epitope via its antigen binding domain, and that the binding entails some complementarity between the antigen binding domain and the epitope. Thus, an antibody is said to “specifically bind” to an epitope when it binds to that epitope via its antigen binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B”, or antibody “A” may be said to bind to epitope “C” with a higher specificity than it has for related epitope “D”. In some embodiments, an antibody or an antibody fragment “has specificity to” an antigen if the antibody or the antigen binding fragment thereof forms a complex with the antigen with a dissociation constant (K_{d}) of 10⁻⁶M or less, 10⁻⁷M or less, 10⁻⁸M or less, 10⁻⁹M or less, or 10⁻¹⁰M or less. In certain embodiments, the specific binding of the antigen binding molecules, e.g., anti-human PD-L1 antibodies or antigen binding fragment thereof, can be shown by the preferential binding of the antigen binding molecules to human PD-L1 expressed on a cell surface using assays described in Examples 4-7, or substantially similar methods.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or Gap, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et a/. (1992) Science 256: 1443-1445. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215:403-410 and Altschul et a/. (1997) Nucleic Acids Res. 25:3389-402.

The terms “therapeutically effective amount,” “pharmacologically effective amount”, and “physiologically effective amount” are used interchangeably to mean the amount of an active agent sufficient to ameliorate at least one symptom of the disease or disorder. For example, for the given parameter, a therapeutically effective amount will show an increase or decrease of at least 5%, 10%, 15%, 20%, 25%, 40%, 50%, 60%, 75%, 80%, 90%, 95%, 99%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control. The precise amount will depend upon numerous factors, e.g., the particular active agent, the components and physical characteristics of the composition, intended patient population, patient considerations, including weight, sex and the like, and can readily be determined by one skilled in the art, based upon the information provided herein or otherwise available in the relevant literature.

The terms “treat” and “treatment” refer to the amelioration of one or more symptoms associated with a disease or disorder. As such, these terms refer to any indicia of success in the therapy or amelioration of an injury, disease, pathology or condition, including prevention or delay of the onset of one or more symptoms of the disease or disorder; lessening of the severity or frequency of one or more symptoms of the disease or disorder; any objective or subjective parameter such as abatement; remission; diminishing of symptoms or making the injury, pathology or condition more tolerable to the patient; slowing in the rate of degeneration or decline; making the final point of degeneration less debilitating; and/or improving a patient's physical or mental well-being. “Treating” and treatment” may also include prophylactic treatment.

The phrases “to a patient in need thereof,” “to a patient in need of treatment,” “to a subject in need thereof,” or “to a subject in need of treatment” includes subjects, such as mammalian subjects, that would benefit from administration of the antibodies of the present disclosure for treatment of a cell proliferative disorder.

The term “variant,” as used herein, refers to a polypeptide, e.g., an antibody, or a polynucleotide, that is derived by incorporation of one or more amino acid or nucleotide insertions, substitutions, or deletions in a precursor polypeptide or polynucleotide (e.g., “parent” polypeptide or polynucleotide). In certain embodiments, a variant polypeptide or polynucleotide has at least about 85% amino acid or nucleotide sequence identity, e.g., about 90%, about 91%, about 92%, about 93%, about 94%, about 95%, about 96%, about 97%, about 98%, about 99%, or about 100%, amino acid or nucleotide sequence identity to the entire amino acid or nucleotide sequence of a parent polypeptide or polynucleotide. A variant of a protein or peptide maintains substantially the similar or identical structures, functions or activities of the protein. For example, a variant of an antibody maintains the function or activities of specifically binding to its antigen and/or modulates, e.g., inhibits, the activities of the antigen. In the case of a polynucleotide, a variant thereof maintains its function or activities of the parent polynucleotide. For example, a variant polynucleotide may encode a protein or peptide that has similar functions or activities of the polypeptide encoded by the parent polynucleotide.

As used herein, the term “PD-L1” refers to the programmed death ligand 1. Unless indicated otherwise, such as by specific reference to human PD-L1, the term “PD-L1” includes all mammalian species of native PD-L1 from, e.g., human, primate, rodent, canine, feline, equine, and bovine. The nucleotide and amino acid sequence of PD-L1 is known and may be found in, for example, GenBank Accession Nos. NP_054862.1, XP_015292694.1, NP_068693.1, XP 014973151.1, NP_068693.1, NP_001178883.1, XP_005615993.1, and G1SUI3-1, the entire contents of each of which are incorporated herein by reference. The following is an exemplary human PD-L1 amino acid sequence:

(SEQ ID NO: 312)
MRIFAVFIFMTYWHLLNAFTVTVPKDLYVVEYGSNMTIECKFPVEKQLDLAALIVYWEMEDKNIIQFVHG
EEDLKVQHSSYRQRARLLKDQLSIGNAALQITDVKLQDAGVYRCMISYGGADYKRITVKVNAPYNKINQR
ILVVDPVTSEHELTCQAEGYPKAEVIWTSSDHQVLSGKTTTTNSKREEKLFNVTSTLRINTTTNEIFYCT
FRRLDPEENHTAELVIPELPLAHPPNERTHLVILGAILLCLGVALTFIFRLRKGRMMDVKKCGIQDTNSK
KQSDTHLEET

An exemplary cynomolgus PD-L1 amino acid sequence is shown below:

(SEQ ID NO: 313)
MRIFAVFIFTIYWHLLNAFTVTVPKDLYVVEYGSNMTIECKFPVEKQLDLTSLIVYWEMEDKNIIQFVHG
EEDLKVQHSNYRQRAQLLKDQLSLGNAALRITDVKLQDAGVYRCMISYGGADYKRITVKVNAPYNKINQR
ILVVDPVTSEHELTCQAEGYPKAEVIWTSSDHQVLSGKTTTTNSKREEKLLNVTSTLRINTTANEIFYCI
FRRLDPEENHTAELVIPELPLALPPNERTHLVILGAIFLLLGVALTFIFYLRKGRMMDMKKCGIRVINSK
KQRGKNIRRNWEVEGKGNKKLKQ

The term “anti-PD-L1 antibody,” or “PD-L1 antibody” refers to an antibody or polypeptide that specifically binds to PD-L1. In certain embodiments, the anti-PD-L1 antibody is able to inhibit PD-L1 biological activity, e.g., via blocking the interaction between PD-1 and PD-L1 and/or downstream signal pathways mediated by PD-L1. Anti-PD-L1 antibodies encompass antibodies or polypeptides contain one or more antigen binding domains in the form of CDRs or variable regions. In certain embodiments, anti-PD-L1 antibodies of the invention block, antagonize, suppress or reduce (to any degree including significantly) PD-L1 biological activity, including downstream events mediated by PD-L1 or PD1, such as PD-L1 binding, PD1/PD-L1 interaction and downstream signaling, inhibition of anti-tumor immune responses, and immunosuppression in immune-compromised disease states. In certain embodiments, anti-PD-L1 antibodies of the invention are able to internalize into PD-L1 expressing tumor cells and elicit antibody drug conjugate induced killing of the tumor cells.

### II. PD-L1 Antibodies and Antigen Binding Proteins

The present invention provides PD-L1 antigen binding molecules that bind specifically to PD-L1. As used herein, the term “antigen binding molecule” refers to a protein, polypeptide or molecular complex comprising or consisting of at least one complementarity determining region (CDR) that alone, or in combination with one or more additional CDRs and/or framework regions (FRs), specifically binds to a particular antigen. In certain embodiments, an antigen binding molecule is an antibody or an antigen binding fragment thereof, as those terms are defined elsewhere herein. In certain embodiments, the antigen binding molecules of the present invention inhibit one or more of its biological functions of PD-L1.

In certain embodiments, the PD-L1 is a human PD-L1. An exemplary human PD-L1 has the amino acid sequence as set forth in SEQ ID NO:. In some embodiments, the PD-L1 is a cynomolgus PD-L1. An exemplary cynomolgus PD-L1 has the amino acid sequence as set forth in SEQ ID NO:.

### 1. The Sequences of Exemplary Antigen Binding Molecules

The PD-L1 antigen binding molecules may be in the form of monoclonal antibodies; one or more polypeptide fragment(s) containing one or more PD-L1 antigen binding domains; or one or more nucleic acids encoding one or more PD-L1 binding domains.

In various exemplary embodiments of the present invention, an antigen binding molecules, e.g., an anti-PD-L1 antibodies or antigen binding fragments thereof, includes (1) a heavy chain variable region, wherein the heavy chain variable region comprises three complementarity determining regions (HCDRs): HCDR1, HCDR2 and HCDR3 and (2) a light chain variable region, wherein the light chain variable region comprises three complementarity determining regions (LCDRs): LCDR1, LCDR2 and LCDR3; wherein the antigen binding molecules binds specifically to human PD-L1. Exemplary HCDR- and LCDR amino acid sequences corresponding to the exemplary anti-human PD-L1 monoclonal antibodies disclosed in the present invention are shown in Tables 1-5.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol, 2001, 309:657-70 (“AHo” numbering scheme); each of which is incorporated by reference in its entirety. Tables 1 and 2 show the sequences of heavy chain CDRs of exemplary antibodies of the invention according to Kabat numbering scheme and IMGT numbering scheme, respectively. Table 3 shows the sequences of heavy chain CDRs of exemplary antibodies of the invention, in which the CDR sequences are defined by combining the CDRs based on Kabat and IMGT numbering schemes. Tables 4 and 5 show the sequences of light chain CDRs of exemplary antibodies of the invention according to Kabat numbering scheme and IMGT numbering scheme.

In certain embodiments, the present invention includes antigen binding molecules, e.g., anti-PD-L1 antibodies or antigen binding fragments thereof, comprising CDRs which are defined based on Kabat and IMGT numbering scheme, or the combination thereof. Accordingly, in certain embodiments, the present invention includes antigen binding molecules, e.g., anti-PD-L1 antibodies or antigen binding fragments thereof, comprising (1) a HCDR1 having an amino acid sequence that is about 80%, 85%, 90%, 95%, 96%, 97%, 98%, 99% to about 100% identical to an amino acid sequence selected from the HCDR1 sequences listed in Tables 1 and 6; (2) a HCDR2 having an amino acid sequence that is about 80%, 85%, 90%, 95%, 96%, 97%, 98%, 99% to about 100% identical to an amino acid sequence that is about 80%, 85%, 90%, 95%, 96%, 97%, 98%, 99% to about 100% identical to an amino acid sequence selected from the HCDR2 sequences listed in Tables 1 and 9; (3) a HCDR3 having an amino acid sequence that is about 80%, 85%, 90%, 95%, 96%, 97%, 98%, 99% to about 100% identical to an amino acid sequence selected from the HCDR3 sequences listed in Tables 1 and 11; (4) a LCDR1 having an amino acid sequence that is about 80%, 85%, 90%, 95%, 96%, 97%, 98%, 99% to about 100% identical to an amino acid sequence selected from the LCDR1 sequences listed in Tables 4 and 13; (5) a LCDR2 having an amino acid sequence that is about 80%, 85%, 90%, 95%, 96%, 97%, 98%, 99% to about 100% identical to an amino acid sequence selected from the LCDR2 sequences listed in Tables 4 and 15; and (6) a LCDR3 having an amino acid sequence that is about 80%, 85%, 90%, 95%, 96%, 97%, 98%, 99% to about 100% identical to an amino acid sequence selected from the LCDR3 sequences listed in Tables 4 and 16.

In certain embodiments, the present invention includes antigen binding molecules, e.g., anti-PD-L1 antibodies or antigen binding fragments thereof, comprising (1) a HCDR1 having an amino acid sequence selected from the HCDR1 sequences that is about 80%, 85%, 90%, 95%, 96%, 97%, 98%, 99% to about 100% identical to an amino acid sequence listed in Tables 2 and 7; (2) a HCDR2 having an amino acid sequence that is about 80%, 85%, 90%, 95%, 96%, 97%, 98%, 99% to about 100% identical to an amino acid sequence selected from the HCDR2 sequences listed in Tables 2 and 10; (3) a HCDR3 having an amino acid sequence that is about 80%, 85%, 90%, 95%, 96%, 97%, 98%, 99% to about 100% identical to an amino acid sequence selected from the HCDR3 sequences listed in Tables 2 and 12; (4) a LCDR1 having an amino acid sequence that is about 80%, 85%, 90%, 95%, 96%, 97%, 98%, 99% to about 100% identical to an amino acid sequence selected from the LCDR1 sequences listed in Table 5 and 14; (5) a LCDR2 having an amino acid sequence that is about 80%, 85%, 90%, 95%, 96%, 97%, 98%, 99% to about 100% identical to an amino acid sequence selected from the LCDR2 sequences listed in Table 5; and (6) a LCDR3 having an amino acid sequence that is about 80%, 85%, 90%, 95%, 96%, 97%, 98%, 99% to about 100% identical to an amino acid sequence selected from the LCDR3 sequences listed in Tables 5 and 16.

In certain embodiments, the present invention includes antigen binding molecules, e.g., anti-PD-L1 antibodies or antigen binding fragments thereof, comprising (1) a HCDR1 having an amino acid sequence that is about 80%, 85%, 90%, 95%, 96%, 97%, 98%, 99% to about 100% identical to an amino acid sequence selected from the HCDR1 sequences listed in Tables 3 and 8; (2) a HCDR2 having an amino acid sequence that is about 80%, 85%, 90%, 95%, 96%, 97%, 98%, 99% to about 100% identical to an amino acid sequence selected from the HCDR2 sequences listed in Tables 3 and 9; (3) a HCDR3 having an amino acid sequence that is about 80%, 85%, 90%, 95%, 96%, 97%, 98%, 99% to about 100% identical to an amino acid sequence selected from the HCDR3 sequences listed in Tables 3 and 11; (4) a LCDR1 having an amino acid sequence that is about 80%, 85%, 90%, 95%, 96%, 97%, 98%, 99% to about 100% identical to an amino acid sequence selected from the LCDR1 sequences listed in Tables 4 and 13; (5) a LCDR2 having an amino acid sequence that is about 80%, 85%, 90%, 95%, 96%, 97%, 98%, 99% to about 100% identical to an amino acid sequence selected from the LCDR2 sequences listed in Tables 4 and 15; and (6) a LCDR3 having an amino acid sequence that is about 80%, 85%, 90%, 95%, 96%, 97%, 98%, 99% to about 100% identical to an amino acid sequence selected from the LCDR3 sequences listed in Tables 4 and 16.

As used herein, a “position” in a CDR refers to the amino acid counted from the N-terminus of the CDR. For example, position 1 or the 1^{st} position in HCDR1 refers to the first amino acid in HCDR1. Accordingly, in clone 27A2, position 1 of HCDR1 based on Kabat numbering scheme is a serine (S).

TABLE 1
Amino Acid Sequences of Heavy Chain CDRs of Exemplary Antibodies
(Kabat Numbering Scheme)

| | | SEQ | | SEQ | | SEQ |
|---|---|---|---|---|---|---|
| CLONE | HCDR1 | ID NO | HCDR2 | ID NO | HCDR3 | ID NO |
| 27A2 | SNHYMS | 1 | SIGVGSGITDYASWAKG | 22 | WTSGGGGFGL | 42 |
| 26G2 | SNHYMC | 2 | CIGVGSGITDYASWAKG | 23 | WTSGGGGFGL | 42 |
| 28A12 | SAYYMC | 3 | CIGVGSGITDYASWAKG | 23 | WTSGGGGFGL | 42 |
| 72G5 | ASYYMC | 4 | CIGIGSGITDYANWAEG | 24 | WTSGGGGFGL | 42 |
| 67B6 | SNYYMC | 5 | CIGIGSYITDYASWAKG | 25 | WTSGGGGFGL | 42 |
| 35C9 | NNHYMC | 6 | CIGIGSGISDYANWAKG | 26 | WTSGGGGFGL | 42 |
| 65B6 | ANYYMC | 7 | CIGIGSGITDYASWAKG | 27 | WTSGGGGFGL | 42 |
| 56H3 | NNYYMS | 8 | SIGIGSGITDYASWAKG | 28 | WTSGGGGFGL | 42 |
| 39C10 | NNYYMC | 9 | CIGIGSGITDYASWAKG | 27 | WTSGGGGFGL | 42 |
| 27C10 | SNYYMC | 5 | CIGIGSGISDYASWAKG | 29 | WTSGGGGFGL | 42 |
| 33H7 | RYFYMS | 11 | SISTGSDITDYASWAKG | 30 | WTSGGGGFGL | 42 |
| 30A6 | SAYWIC | 12 | CISTGSAITDYASWAKG | 31 | WTSGGGGFGL | 42 |
| 32E2 | SSYYMS | 13 | SISGGVTDNAYYASWAK | 32 | DSSSGYFFLL | 43 |
| | | | G | | | |
| 28B3 | SSYYMI | 14 | YVYTGSGNTWYASWAKG | 33 | ASGADGVYDWGW | 44 |
| | | | | | DI | |
| 33E8 | SGYDMS | 15 | SIFTTSGSTWYANWAKG | 34 | TKDGVGSFYMNI | 45 |
| 53G7 | SGYDMC | 16 | CIFTGSGSTWYANWAKG | 35 | TRDGAGSFYMNL | 46 |
| 74A9 | STYALG | 17 | SISIGGATYYASWAKG | 36 | ARNVDSIYLDAF | 47 |
| | | | | | HT | |
| 38D10 | STYAMG | 18 | SISIGGATYFATWAKG | 37 | ARNVDIIYLNAF | 48 |
| | | | | | DI | |
| 55H3/ | GDYYMS | 19 | SIYTGSLNSYYATWAKG | 38 | RNKNADYGHFDL | 49 |
| 76D6 | | | | | | |
| 56E5 | GDYYMS | 19 | SIYTGSLNTYYATWAKG | 39 | RHKNADYGHFDL | 50 |
| 57B10 | DDYYMS | 20 | SIYTGSLNTYYATWAKG | 39 | RNKNADWGHFDL | 51 |
| 57F11 | GDYYMS | 19 | SIYTGSLNTYYATWAKG | 39 | RTKNGDYGYFDL | 52 |
| 62E6 | GDYYMS | 19 | SIYTGSLNIYYATWAKG | 40 | RNKNADYGHEDL | 49 |
| 69D10 | GDWYMS | 21 | SIYSGSLNTYYATWAKG | 41 | RNKNADYGHFDL | 49 |
| 73C8 | GDYYMS | 19 | SIYTGSLNTYYATWAKG | 39 | RNKNADYGHFDL | 49 |
| 74F4 | GDYYMS | 19 | SIYTGSLNTYYATWAKG | 39 | RNKNADYGHFDL | 49 |

TABLE 2
Amino Acid Sequences of Heavy Chain CDRS of Exemplary Antibodies
(IMGT Numbering Scheme)

| | | SEQ | | SEQ | | SEQ |
|---|---|---|---|---|---|---|
| Clone | HCDR1 | ID NO | HCDR2 | ID NO | HCDR3 | ID NO |
| 27A2 | GFSFSSNHY | 144 | IGVGSGI | 161 | ARWTSGGGGFGL | 172 |
| 26G2 | GFSFSSNHY | 144 | IGVGSGI | 161 | ARWTSGGGGFGL | 172 |
| 28A12 | GFSFSSAYY | 145 | IGVGSGI | 161 | ARWTSGGGGFGL | 172 |
| 72G5 | GFSFSASYY | 146 | IGIGSGI | 162 | ARWTSGGGGFGL | 172 |
| 67B6 | EFSFSSNYY | 147 | IGIGSGI | 162 | ARWTSGGGGFGL | 172 |
| 35C9 | AFSFNNNHY | 148 | IGIGSGI | 162 | ARWTSGGGGFGL | 172 |
| 65B6 | EFSFGANYY | 149 | IGIGSGI | 162 | ARWTSGGGGFGL | 172 |
| 56H3 | EFSFSNNYY | 150 | IGIGSGI | 162 | ARWTSGGGGFGL | 172 |
| 39C10 | EFSFSNNYY | 150 | IGIGSGI | 162 | ARWTSGGGGFGL | 172 |
| 27C10 | EFSFSSNYY | 147 | IGIGSGI | 162 | ARWTSGGGGFGL | 172 |
| 33H7 | GFDLSRYFY | 151 | ISTGSDI | 163 | ARWTSGGGGFGL | 172 |
| 30A6 | GFDFSSAYW | 152 | ISTGSAI | 164 | ARWTSGGGGFGL | 172 |
| 32E2 | GFSFSSSYY | 153 | ISGGVTD | 165 | ARDSSSGYFFLL | 173 |
| 28B3 | GFSLSSYY | 154 | VYTGSGN | 166 | ASGADGVYDWGWDI | 44 |
| 33E8 | GFYFSSGYD | 155 | IFTTSGS | 167 | TKDGVGSFYMNL | 45 |
| 53G7 | GFYFSSGYD | 155 | IFTGSGS | 168 | TRDGAGSFYMNL | 46 |
| 74A9 | GFSLSTYA | 156 | ISIGGAT | 169 | ARNVDSIYLDAFHT | 47 |
| 38D10 | GIDLSTYA | 157 | ISIGGAT | 169 | ARNVDIIYLNAFDI | 48 |
| 55H3/ | GFSFSGDYY | 158 | IYTGSLN | 170 | ARRNKNADYGHFDL | 174 |
| 76D6 | | | | | | |
| 56E5 | GFSFSGDYY | 158 | IYTGSLN | 170 | ARRHKNADYGHFDL | 175 |
| 57B10 | GFSFSDDYY | 159 | IYTGSLN | 170 | ARRNKNADWGHFDL | 176 |
| 57F11 | GFSFSGDYY | 158 | IYTGSLN | 170 | ARRTKNGDYGYFDL | 177 |
| 62E6 | GFSFSGDYY | 158 | IYTGSLN | 170 | ARRNKNADYGHFDL | 174 |
| 69D10 | GFSFSGDWY | 160 | IYSGSLN | 171 | ARRNKNADYGHFDL | 174 |
| 73C8 | GFSFSGDYY | 158 | IYTGSLN | 170 | ARRNKNADYGHFDL | 174 |
| 74F4 | GFSFSGDYY | 158 | IYTGSLN | 170 | ARRNKNADYGHFDL | 174 |

TABLE 3
Amino Acid Sequences of Heavy Chain CDRs of Exemplary Antibodies
(Combing Kabat and IMGT Numbering Scheme)

| | | SEQ ID | | SEQ ID | | SEQ ID |
|---|---|---|---|---|---|---|
| Clone | HCDR1 | NO | HCDR2 | NO | HCDR3 | NO |
| 27A2 | GFSFSSNHYMS | 178 | SIGVGSGITDYASWAKG | 22 | ARWTSGGGGFGL | 172 |
| 26G2 | GFSFSSNHYMC | 179 | CIGVGSGITDYASWAKG | 23 | ARWTSGGGGFGL | 172 |
| 28A12 | GFSFSSAYYMC | 180 | CIGVGSGITDYASWAKG | 23 | ARWTSGGGGFGL | 172 |
| 72G5 | GFSFSASYYMC | 181 | CIGIGSGITDYANWAEG | 24 | ARWTSGGGGFGL | 172 |
| 67B6 | EFSFSSNYYMC | 182 | CIGIGSYITDYASWAKG | 25 | ARWTSGGGGFGL | 172 |
| 35C9 | AFSFNNNHYMC | 183 | CIGIGSGISDYANWAKG | 26 | ARWTSGGGGFGL | 172 |
| 65B6 | EFSFGANYYMC | 184 | CIGIGSGITDYASWAKG | 27 | ARWTSGGGGFGL | 172 |
| 56H3 | EFSFSNNYYMS | 185 | SIGIGSGITDYASWAKG | 28 | ARWTSGGGGFGL | 172 |
| 39C10 | EFSFSNNYYMC | 186 | CIGIGSGITDYASWAKG | 27 | ARWTSGGGGFGL | 172 |
| 27C10 | EFSFSSNYYMC | 182 | CIGIGSGISDYASWAKG | 29 | ARWTSGGGGFGL | 172 |
| 33H7 | GFDLSRYFYMS | 187 | SISTGSDITDYASWAKG | 30 | ARWTSGGGGFGL | 172 |
| 30A6 | GFDFSSAYWIC | 188 | CISTGSAITDYASWAKG | 31 | ARWTSGGGGFGL | 172 |
| 32E2 | GFSFSSSYYMS | 189 | SISGGVTDNAYYASWAKG | 32 | ARDSSSGYFFLL | 173 |
| 28B3 | GFSLSSYYMI | 190 | YVYTGSGNTWYASWAKG | 33 | ASGADGVYDWGWDI | 44 |
| 33E8 | GFYFSSGYDMS | 191 | SIFTTSGSTWYANWAKG | 34 | TKDGVGSFYMNL | 45 |
| 53G7 | GFYFSSGYDMC | 192 | CIFTGSGSTWYANWAKG | 35 | TRDGAGSFYMNL | 46 |
| 74A9 | GFSLSTYALG | 193 | SISIGGATYYASWAKG | 36 | ARNVDSIYLDAFHT | 47 |
| 38D10 | GIDLSTYAMG | 194 | SISIGGATYFATWAKG | 37 | ARNVDIIYLNAFDI | 48 |
| 55H3/ | GFSFSGDYYMS | 195 | SIYTGSLNSYYATWAKG | 38 | ARRNKNADYGHEDL | 174 |
| 76D6 | | | | | | |
| 56E5 | GFSFSGDYYMS | 195 | SIYTGSLNTYYATWAKG | 39 | ARRHKNADYGHFDL | 175 |
| 57B10 | GFSFSDDYYMS | 196 | SIYTGSLNTYYATWAKG | 39 | ARRNKNADWGHFDL | 176 |
| 57F11 | GFSFSGDYYMS | 195 | SIYTGSLNTYYATWAKG | 39 | ARRTKNGDYGYFDL | 177 |
| 62E6 | GFSFSGDYYMS | 195 | SIYTGSLNIYYATWAKG | 40 | ARRNKNADYGHEDL | 174 |
| 69D10 | GFSFSGDWYMS | 197 | SIYSGSLNTYYATWAKG | 41 | ARRNKNADYGHEDL | 174 |
| 73C8 | GFSFSGDYYMS | 195 | SIYTGSLNTYYATWAKG | 39 | ARRNKNADYGHEDL | 174 |
| 74F4 | GFSFSGDYYMS | 195 | SIYTGSLNTYYATWAKG | 39 | ARRNKNADYGHEDL | 174 |

TABLE 4
Amino Acid Sequences Light Chain CDRs of Exemplary Antigen Binding Molecules
(Kabat Numbering Scheme)

| | | SEQ | | SEQ | | SEQ |
|---|---|---|---|---|---|---|
| Clone | LCDR1 | ID NO | LCDR2 | ID NO | LCDR3 | ID NO |
| 27A2 | QSSQTIYSDYLF | 53 | QTSTLAS | 69 | QGYYSGYIWT | 83 |
| 26G2 | QSSQTIYSDYLF | 53 | QTSTLAS | 69 | QGYYSGYIWT | 83 |
| 28A12 | QSSQSVYSNYLS | 54 | YASTLAS | 70 | QGYYSGYIWT | 83 |
| 72G5 | QSSQSVYSNYLA | 55 | GASTLAS | 71 | QGYYSGYIWT | 83 |
| 67B6 | QSSQTIYSDYLS | 56 | QTSTLAS | 69 | QGYYSGYIWT | 83 |
| 35C9 | QSSQNIYSDYLS | 57 | GASTLAS | 71 | QGYYSGYIWT | 83 |
| 65B6 | QSSQTIYSDYLS | 56 | QTSTLAS | 69 | QGYYSGYIWT | 83 |
| 56H3 | QSSQTIYSDYLF | 53 | QTSTLAS | 69 | QGYYSGYIWT | 83 |
| 39C10 | QSSQTIYSDYLF | 53 | QTSTLAS | 69 | QGYYSGYIWT | 83 |
| 27C10 | QSSQTIYSDYLF | 53 | QTSTLAS | 69 | QGYYSGYIWT | 83 |
| 33H7 | QSSQSIYNNYLS | 58 | STATLAS | 72 | QGYYSGYIWT | 83 |
| 30A6 | QSSQSVYSNYLC | 59 | DASTLAS | 73 | QGYYSGYIWT | 83 |
| 32E2 | QASQNIYSNLA | 60 | GASNLRS | 74 | QEGYSIGNVDNP | 84 |
| 28B3 | QASQSIYSLLA | 61 | GASNLES | 75 | QNNYDSGRIYGLA | 85 |
| 33E8 | QASQTIGSNLA | 62 | KAFTLAS | 76 | QQGATRINIDNA | 86 |
| 53G7 | QASGTIGSNLA | 63 | KTFTLAS | 77 | QQGASRINIDNA | 87 |
| 74A9 | QASQNIYNNLA | 64 | RASTLAS | 78 | QTYYLTTTTNA | 88 |
| 38D10 | QASQNIYNNLA | 64 | SSSTLAS | 79 | QTYYLTSTINA | 89 |
| 55H3/ | QASESINDRLA | 65 | AASTLAS | 80 | QQGWTVSSLDNA | 90 |
| 76D6 | | | | | | |
| 56E5 | QASQSINDRLA | 66 | GASTLAS | 71 | QQGWTVSSLDNA | 90 |
| 57B10 | QASQSISTALA | 67 | AASTLAS | 80 | QQGWTVSSLDNA | 90 |
| 57F11 | QASQSINDRLA | 66 | AASTLAS | 80 | QQGWTVSSLENA | 91 |
| 62E6 | QASQSINDRLA | 66 | GASTLAS | 71 | QQGWTVSSLDNA | 90 |
| 69D10 | QASQSIGNALA | 68 | ATSTLAS | 82 | QQGWTVSSLDNA | 90 |
| 73C8 | QASQSINDRLA | 66 | AASTLAS | 80 | QQGWTVSSLDNA | 90 |
| 74F4 | QASQSINDRLA | 66 | GASTLAS | 71 | QQGWTVSSLDNA | 90 |

TABLE 5
Amino Acid Sequences Light Chain CDRs of Exemplary Antigen Binding Molecules
(IMGT Numbering Scheme)

| | | SEQ | | SEQ | | SEQ ID |
|---|---|---|---|---|---|---|
| Clone | LCDR1 | ID NO | LCDR2 | ID NO | LCDR3 | NO |
| 27A2 | QTIYSDY | 198 | QTS | 211 | QGYYSGYIWT | 83 |
| 26G2 | QTIYSDY | 198 | QTS | 211 | QGYYSGYIWT | 83 |
| 28A12 | QSVYSNY | 199 | YAS | 212 | QGYYSGYIWT | 83 |
| 72G5 | QSVYSNY | 199 | GAS | 213 | QGYYSGYIWT | 83 |
| 67B6 | QTIYSDY | 198 | QTS | 211 | QGYYSGYIWT | 83 |
| 35C9 | QNIYSDY | 200 | GAS | 213 | QGYYSGYIWT | 83 |
| 65B6 | QTIYSDY | 198 | QTS | 211 | QGYYSGYIWT | 83 |
| 56H3 | QTIYSDY | 198 | QTS | 211 | QGYYSGYIWT | 83 |
| 39C10 | QTIYSDY | 198 | QTS | 211 | QGYYSGYIWT | 83 |
| 27C10 | QTIYSDY | 198 | QTS | 211 | QGYYSGYIWT | 83 |
| 33H7 | QSIYNNY | 201 | STA | 214 | QGYYSGYIWT | 83 |
| 30A6 | QSVYSNY | 199 | DAS | 215 | QGYYSGYIWT | 83 |
| 32E2 | QNIYSN | 202 | GAS | 213 | QEGYSIGNVDNP | 84 |
| 28B3 | QSIYSL | 203 | GAS | 213 | QNNYDSGRIYGLA | 85 |
| 33E8 | QTIGSN | 204 | KAF | 216 | QQGATRINIDNA | 86 |
| 53G7 | GTIGSN | 205 | KTF | 217 | QQGASRINIDNA | 87 |
| 74A9 | QNIYNN | 206 | RAS | 218 | QTYYLTTTTNA | 88 |
| 38D10 | QNIYNN | 206 | SSS | 219 | QTYYLTSTINA | 89 |
| 55H3/ | ESINDR | 207 | AAS | 220 | QQGWTVSSLDNA | 90 |
| 76D6 | | | | | | |
| 56E5 | QSINDR | 208 | GAS | 213 | QQGWTVSSLDNA | 90 |
| 57B10 | QSISTA | 209 | AAS | 220 | QQGWTVSSLDNA | 90 |
| 57F11 | QSINDR | 208 | AAS | 220 | QQGWTVSSLENA | 91 |
| 62E6 | QSINDR | 208 | GAS | 213 | QQGWTVSSLDNA | 90 |
| 69D10 | QSIGNA | 210 | ATS | 221 | QQGWTVSSLDNA | 90 |
| 73C8 | QSINDR | 208 | AAS | 220 | QQGWTVSSLDNA | 90 |
| 74F4 | QSINDR | 208 | GAS | 213 | QQGWTVSSLDNA | 90 |

Tables 6-16 show the consensus sequences for several CDRs in several exemplary antibodies of the present disclosure.

TABLE 6
Consensus Sequences of Heavy Chain CDR1 in Exemplary Antigen
Binding Molecules (Kabat Numbering Scheme)

| Clone | HCDR1 | SEQ ID NO: |
|---|---|---|
| 27A2 | SNHYMS | 1 |
| 26G2 | SNHYMC | 2 |
| 35C9 | NNHYMC | 6 |
| N/A | X1-N-H-Y-M-X2 | 301 |
| 28A12 | SAYYMC | 3 |
| 72G5 | ASYYMC | 4 |
| 67B6 | SNYYMC | 5 |
| 65B6 | ANYYMC | 7 |
| 56H3 | NNYYMS | 8 |
| 39C10 | NNYYMC | 9 |
| 27C10 | SNYYMC | 5 |
| N/A | X3-X4-Y-Y-M-X5 (SEQ ID NO: 302) | 302 |
| | or X3-X4-Y-Y-M-C (SEQ ID NO: | 303 |
| | 303) (Excluding clone 56H3) | |
| 33E8 | SGYDMS | 15 |
| 53G7 | SGYDMC | 16 |
| N/A | S-G-Y-D-M-X6 | 304 |
| 74A9 | STYALG | 17 |
| 38D10 | STYAMG | 18 |
| N/A | S-T-Y-A-X7-G | 305 |
| 55H3/76D6 | GDYYMS | 19 |
| 56E5 | GDYYMS | 19 |
| 57B10 | DDYYMS | 20 |
| 57F11 | GDYYMS | 19 |
| 62E6 | GDYYMS | 19 |
| 69D10 | GDWYMS | 21 |
| 73C8 | GDYYMS | 19 |
| 74F4 | GDYYMS | 19 |
| N/A | X8-D-X9-Y-M-S | 306 |

As used herein, X1 is N or 5, X2 is C or S, X3 is A, N, or 5, X4 is A, N or 5, X5 is C or S, X6 is C or S, X7 is L or M, X8 is G or D, and X9 is W or Y.

TABLE 7
Consensus Sequences of Heavy Chain CDR1 in Exemplary Antigen
Binding Molecules (IMGT Numbering Scheme)

| Clone | HCDR1 | SEQ ID NO: |
|---|---|---|
| 27A2 | GFSFSSNHY | 144 |
| 26G2 | GFSFSSNHY | 144 |
| 28A12 | GFSFSSAYY | 145 |
| 72G5 | GFSFSASYY | 146 |
| N/A | G-F-S-F-S-X10-X11-X12-Y | 222 |
| 67B6 | EFSFSSNYY | 147 |
| 65B6 | EFSFGANYY | 149 |
| 56H3 | EFSFSNNYY | 150 |
| 39C10 | EFSFSNNYY | 150 |
| 27C10 | EFSFSSNYY | 147 |
| N/A | E-F-S-F-X13-X14-N-Y-Y | 223 |
| 74A9 | GFSLSTYA | 156 |
| 38D10 | GIDLSTYA | 157 |
| N/A | G-X15-X16-L-S-T-Y-A | 224 |
| 55H3/76D6 | GFSFSGDYY | 158 |
| 56E5 | GFSFSGDYY | 158 |
| 57B10 | GFSFSDDYY | 159 |
| 57F11 | GFSFSGDYY | 158 |
| 62E6 | GFSFSGDYY | 158 |
| 69D10 | GFSFSGDWY | 160 |
| 73C8 | GFSFSGDYY | 158 |
| 74F4 | GFSFSGDYY | 158 |
| N/A | G-F-S-F-S-X17-D-X18-Y | 225 |

As used herein, X10 is A or 5, X11 is A, N, or 5, X12 is H or Y, X 13 is G or S, X14 is A, N, or S, X15 is F or I, X16 is D or S, X17 is D or G, and X18 is W or Y.

TABLE 8
Consensus Sequences of Heavy Chain CDR1 in Exemplary Antigen
Binding Molecules (Combing Kabat and IMGT Numbering Scheme)

| Clone | HCDR1 | SEQ ID NO: |
|---|---|---|
| 27A2 | GFSFSSNHYMS | 178 |
| 26G2 | GFSFSSNHYMC | 179 |
| 28A12 | GFSFSSAYYMC | 180 |
| 72G5 | GFSFSASYYMC | 181 |
| N/A | G-F-S-F-S-X19-X20-X21-Y-M-X22 | 307 |
| 67B6 | EFSFSSNYYMC | 182 |
| 65B6 | EFSFGANYYMC | 184 |
| 56H3 | EFSFSNNYYMS | 185 |
| 39C10 | EFSFSNNYYMC | 186 |
| 27C10 | EFSFSSNYYMC | 182 |
| N/A | E-F-S-F-X23-X24-N-Y-Y-M-X25 | 308 |
| 33E8 | GFYFSSGYDMS | 191 |
| 53G7 | GFYFSSGYDMC | 192 |
| N/A | G-F-Y-F-S-S-G-Y-D-M-X26 | 309 |
| 74A9 | GFSLSTYALG | 193 |
| 38D10 | GIDLSTYAMG | 194 |
| N/A | G-X27-X28-L-S-T-Y-A-X29-G | 310 |
| 55H3/76D6 | GFSFSGDYYMS | 195 |
| 56E5 | GFSFSGDYYMS | 195 |
| 57B10 | GFSFSDDYYMS | 196 |
| 57F11 | GFSFSGDYYMS | 195 |
| 62E6 | GFSFSGDYYMS | 195 |
| 69D10 | GFSFSGDWYMS | 197 |
| 73C8 | GFSFSGDYYMS | 195 |
| 74F4 | GFSFSGDYYMS | 195 |
| N/A | G-F-S-F-S-X30-D-X31-Y-M-S | 311 |

As used herein, X19 is A or S, X20 is A, N, or S, X21 is H or Y, X22 is C or S, X23 is G or S, X24 is A, N, or 5, X25 is C or 5, X26 is C or 5, X27 is F or I, X28 is D or S, X29 is L or M, X30 is D or G, and X31 is W or Y.

TABLE 9
Consensus Sequences of Heavy Chain CDR2 in Exemplary Antigen
Binding Molecules (Kabat Numbering Scheme)

| Clone | HCDR2 | SEQ ID NO: |
|---|---|---|
| 27A2 | SIGVGSGITDYASWAKG | 22 |
| 26G2 | CIGVGSGITDYASWAKG | 23 |
| 28A12 | CIGVGSGITDYASWAKG | 23 |
| 72G5 | CIGIGSGITDYANWAEG | 24 |
| 67B6 | CIGIGSYITDYASWAKG | 25 |
| 35C9 | CIGIGSGISDYANWAKG | 26 |
| 65B6 | CIGIGSGITDYASWAKG | 27 |
| 56H3 | SIGIGSGITDYASWAKG | 28 |
| 39C10 | CIGIGSGITDYASWAKG | 27 |
| 27C10 | CIGIGSGISDYASWAKG | 29 |
| 33H7 | SISTGSDITDYASWAKG | 30 |
| 30A6 | CISTGSAITDYASWAKG | 31 |
| N/A | X32-I-X33-X34-G-S-X35-I-X36-D-Y-A-X37- | 226 |
| | W-A-K-G | |
| 33E8 | SIFTTSGSTWYANWAKG | 34 |
| 53G7 | CIFTGSGSTWYANWAKG | 35 |
| N/A | X38-I-F-T-X39-S-G-S-T-W-Y-A-N-W-A-K-G | 227 |
| 74A9 | SISIGGATYYASWAKG | 36 |
| 38D10 | SISIGGATYFATWAKG | 37 |
| N/A | S-I-S-I-G-G-A-T-Y-X40-A-X41-W-A-K-G | 228 |
| 55H3/76D6 | SIYTGSLNSYYATWAKG | 38 |
| 56E5 | SIYTGSLNTYYATWAKG | 39 |
| 57B10 | SIYTGSLNTYYATWAKG | 39 |
| 57F11 | SIYTGSLNTYYATWAKG | 39 |
| 62E6 | SIYTGSLNIYYATWAKG | 40 |
| 69D10 | SIYSGSLNTYYATWAKG | 41 |
| 73C8 | SIYTGSLNTYYATWAKG | 39 |
| 74F4 | SIYTGSLNTYYATWAKG | 39 |
| N/A | S-I-Y-X42-G-S-L-N-X43-Y-Y-A-T-W-A-K-G | 229 |

As used herein, X32 is C or S, X33 is G or S, X34 is I, T, or V, X35 is A, D, G, or Y, X36 is S or T, X37 is N or 5, X38 is C or S, X39 is G or T, X40 is F or Y, X 41 is S or T, X42 is S or T, and X43 is I, S or T.

TABLE 10
Consensus Sequences of Heavy Chain CDR2 in Exemplary Antigen
Binding Molecules (IMGT Numbering Scheme)

| Clone | HCDR2 | SEQ ID NO: |
|---|---|---|
| 27A2 | IGVGSGI | 161 |
| 26G2 | IGVGSGI | 161 |
| 28A12 | IGVGSGI | 161 |
| 72G5 | IGIGSGI | 162 |
| 67B6 | IGIGSGI | 162 |
| 35C9 | IGIGSGI | 162 |
| 65B6 | IGIGSGI | 162 |
| 56H3 | IGIGSGI | 162 |
| 39C10 | IGIGSGI | 162 |
| 27C10 | IGIGSGI | 162 |
| 33H7 | ISTGSDI | 163 |
| 30A6 | ISTGSAI | 164 |
| N/A | I-X44-X45-G-S-X46-I (SEQ ID NO: 230) or | 230 |
| | I-G-X47-G-S-G-I (SEQ ID NO: 231) (excluding | 231 |
| | clones 33H7 and 30A6) or I-S-T-G-S-X48-I | 232 |
| | (SEQ ID NO: 232) (clones 33H7 and 30A6 | |
| | only) | |
| 33E8 | IFTTSGS | 167 |
| 53G7 | IFTGSGS | 168 |
| N/A | I-F-T-X49-S-G-S | 233 |
| 55H3/76D6 | IYTGSLN | 170 |
| 56E5 | IYTGSLN | 170 |
| 57B10 | IYTGSLN | 170 |
| 57F11 | IYTGSLN | 170 |
| 62E6 | IYTGSLN | 170 |
| 69D10 | IYSGSLN | 171 |
| 73C8 | IYTGSLN | 170 |
| 74F4 | IYTGSLN | 170 |
| N/A | I-Y-X50-G-S-L-N | 234 |

As used herein, X44 is G or S, X45 is I, T, or V, X46 is A, D, or G, X47 is I or V, X48 is A or D, X49 is G or T, and X50 is S or T.

TABLE 11
Consensus Sequences of Heavy Chain CDR3 in Exemplary Antigen
Binding Molecules (Kabat Numbering Scheme)

| Clone | HCDR3 | SEQ ID NO: |
|---|---|---|
| 33E8 | TKDGVGSFYMNL | 45 |
| 53G7 | TRDGAGSFYMNL | 46 |
| N/A | T-X51-D-G-X52-G-S-F-Y-M-N-L | 235 |
| 74A9 | ARNVDSIYLDAFHT | 47 |
| 38D10 | ARNVDIIYLNAFDI | 48 |
| N/A | A-R-N-V-D-X53-I-Y-L-X54-A-F-X55-X56 | 236 |
| 55H3/76D6 | RNKNADYGHFDL | 49 |
| 56E5 | RHKNADYGHFDL | 50 |
| 57B10 | RNKNADWGHFDL | 51 |
| 57F11 | RTKNGDYGYFDL | 52 |
| 62E6 | RNKNADYGHFDL | 49 |
| 69D10 | RNKNADYGHFDL | 49 |
| 73C8 | RNKNADYGHFDL | 49 |
| 74F4 | RNKNADYGHFDL | 49 |
| N/A | R-X57-K-N-X58-D-X59-G-X60-F-D-L | 237 |

As used herein, X51 is K or R, X52 is A or V, X53 is I or S, X54 is D or N, X55 is D or H, X56 is I or T, X57 is H, N, or T, X58 is A or G, X59 is W or Y, and X60 is H or Y.

TABLE 12
Consensus Sequences of Heavy Chain CDR3 in Exemplary Antigen
Binding Molecules (IMGT Numbering Scheme)

| Clone | HCDR3 | SEQ ID NO: |
|---|---|---|
| 33E8 | TKDGVGSFYMNL | 45 |
| 53G7 | TRDGAGSFYMNL | 46 |
| N/A | T-X51-D-G-X52-G-S-F-Y-M-N-L | 235 |
| 74A9 | ARNVDSIYLDAFHT | 47 |
| 38D10 | ARNVDIIYLNAFDI | 48 |
| N/A | A-R-N-V-D-X53-I-Y-L-X54-A-F-X55-X56 | 236 |
| 55H3/76D6 | ARRNKNADYGHFDL | 174 |
| 56E5 | ARRHKNADYGHFDL | 175 |
| 57B10 | ARRNKNADWGHFDL | 176 |
| 57F11 | ARRTKNGDYGYFDL | 177 |
| 62E6 | ARRNKNADYGHFDL | 174 |
| 69D10 | ARRNKNADYGHFDL | 174 |
| 73C8 | ARRNKNADYGHFDL | 174 |
| 74F4 | ARRNKNADYGHFDL | 174 |
| N/A | A-R-R-X61-K-N-X62-D-X63-G-X64-F-D-L | 238 |

As used herein, X61 is H, N, or T, X62 is A or G, X63 is W or Y, and X64 is H or Y.

TABLE 13
Consensus Sequences of Light Chain CDR1 in Exemplary Antigen Binding Molecules
(Kabat Numbering Scheme)

| Clone | LCDR1 | SEQ ID NO: |
|---|---|---|
| 27A2 | QSSQTIYSDYLF | 53 |
| 26G2 | QSSQTIYSDYLF | 53 |
| 28A12 | QSSQSVYSNYLS | 54 |
| 72G5 | QSSQSVYSNYLA | 55 |
| 67B6 | QSSQTIYSDYLS | 56 |
| 35C9 | QSSQNIYSDYLS | 57 |
| 65B6 | QSSQTIYSDYLS | 56 |
| 56H3 | QSSQTIYSDYLF | 53 |
| 39C10 | QSSQTIYSDYLF | 53 |
| 27C10 | QSSQTIYSDYLF | 53 |
| 33H7 | QSSQSIYNNYLS | 58 |
| 30A6 | QSSQSVYSNYLC | 59 |
| N/A | Q-S-S-Q-X65-X66-Y-X67-X68-Y-L-X69 (SEQ ID | 239 |
| | NO: 239) or Q-S-S-Q-X70-I-Y-S-D-Y-L-X71 | 240 |
| | (SEQ ID NO: 240) (clones with a N or T at | 241 |
| | 5^{th} position) or Q-S-S-Q-S-X72-Y-X73-N-Y- | |
| | L-X74 (SEQ ID NO: 241) (clones with an S at | |
| | 5^{th} position) | |
| 33E8 | QASQTIGSNLA | 62 |
| 53G7 | QASGTIGSNLA | 63 |
| N/A | Q-A-S-X75-T-I-G-S-N-L-A | 242 |
| 55H3/76D6 | QASESINDRLA | 65 |
| 56E5 | QASQSINDRLA | 66 |
| 57B10 | QASQSISTALA | 67 |
| 57F11 | QASQSINDRLA | 66 |
| 62E6 | QASQSINDRLA | 66 |
| 69D10 | QASQSIGNALA | 68 |
| 73C8 | QASQSINDRLA | 66 |
| 74F4 | QASQSINDRLA | 66 |
| N/A | Q-A-S-X76-S-I-X77-X78-X79-L-A (SEQ ID NO: | 243 |
| | 243) or Q-A-S-X80-S-I-N-D-R-L-A (SEQ ID | |
| | NO: 244) (excluding clones 57B10 and | |
| | 69D10) or Q-A-S-Q-S-I-X81-X82-A-L-A (SEQ | |
| | ID NO: 245) (clones 57B10 and 69D10 only) | |

As used herein, X65 is N, 5, or T, X66 is I or V, X67 is N or 5, X68 is D or N, X69 is A, C, F, or S, X70 is N or T, X71 is F or S, X72 is I or V, X73 is N or S, X74 is A, C, or S, X75 is G or Q, X76 is E or Q, X77 is G, N, or 5, X78 is D, N, or T, X79 is A or R, X80 is E or Q, X81 is G or S, and X82 is N or T.

TABLE 14
Consensus Sequences of Light Chain CDR1 in Exemplary Antigen Binding Molecules
(IMGT Numbering Scheme)

| Clone | LCDR1 | SEQ ID NO: |
|---|---|---|
| 27A2 | QTIYSDY | 198 |
| 26G2 | QTIYSDY | 198 |
| 28A12 | QSVYSNY | 199 |
| 72G5 | QSVYSNY | 199 |
| 67B6 | QTIYSDY | 198 |
| 35C9 | QNIYSDY | 246 |
| 65B6 | QTIYSDY | 198 |
| 56H3 | QTIYSDY | 198 |
| 39C10 | QTIYSDY | 198 |
| 27C10 | QTIYSDY | 198 |
| 33H7 | QSIYNNY | 247 |
| 30A6 | QSVYSNY | 199 |
| N/A | Q-X83-X84-Y-X85-X86-Y (SEQ ID No: 248) or | 248 |
| | Q-X87-I-Y-S-D-Y (SEQ ID NO: 249) (clones | 249 |
| | with a N or T at 2nd position) or Q-S-X88- | 250 |
| | Y-X89-N-Y (SEQ ID NO: 250) (clones with an | |
| | S at 2^{nd} position) | |
| 33E8 | QTIGSN | 204 |
| 53G7 | GTIGSN | 205 |
| N/A | X90-T-I-G-S-N | 251 |
| 55H3/76D6 | ESINDR | 252 |
| 56E5 | QSINDR | 208 |
| 57B10 | QSISTA | 209 |
| 57F11 | QSINDR | 208 |
| 62E6 | QSINDR | 208 |
| 69D10 | QSIGNA | 263 |
| 73C8 | QSINDR | 208 |
| 74F4 | QSINDR | 208 |
| N/A | X91-S-I-X92-X93-X94 (SEQ ID NO: 253) or | 253 |
| | X95-S-I-N-D-R (SEQ ID NO: 254) (excluding | 254 |
| | clones 57B10 and 69D10) or Q-S-I-X96-X97-A | 255 |
| | (SEQ ID NO: 255) (clones 57B10 and 69D10 | |
| | only) | |

As used herein, X83 is N, 5, or T, X84 is 1, or V, X85 is N or 5, X86 is D or N, X87 is N or T, X88 is I or V, X89 is N or S, X90 is G or Q, X91 is E or Q, X92 is G, N, or S, X93 is D, N, or T, X94 is A or R, X95 is E or Q, X96 is G or S, and X97 is N or T.

TABLE 15
Consensus Sequences of Light Chain CDR2 in Exemplary Antigen Binding Molecules
(Kabat Numbering Scheme)

| Clone | LCDR2 | SEQ ID NO: |
|---|---|---|
| 27A2 | QTSTLAS | 69 |
| 26G2 | QTSTLAS | 69 |
| 28A12 | YASTLAS | 70 |
| 72G5 | GASTLAS | 71 |
| 67B6 | QTSTLAS | 69 |
| 35C9 | GASTLAS | 71 |
| 65B6 | QTSTLAS | 69 |
| 56H3 | QTSTLAS | 69 |
| 39C10 | QTSTLAS | 69 |
| 27C10 | QTSTLAS | 69 |
| 33H7 | STATLAS | 72 |
| 30A6 | DASTLAS | 73 |
| N/A | X98-X99-X100-T-L-A-S (SEQ ID NO: 256) or | 256 |
| | X101-X102-S-T-L-A-S (SEQ ID NO: 257) | 257 |
| | (excluding clone 33H7) | |
| 33E8 | KAFTLAS | 76 |
| 53G7 | KTFTLAS | 77 |
| N/A | K-X103-F-T-L-A-S | 258 |
| 74A9 | RASTLAS | 78 |
| 38D10 | SSSTLAS | 79 |
| N/A | X104-X105-S-T-L-A-S | 259 |
| 55H3/76D6 | AASTLAS | 80 |
| 56E5 | GASTLAS | 71 |
| 57B10 | AASTLAS | 80 |
| 57F11 | AASTLAS | 80 |
| 62E6 | GASTLAS | 71 |
| 69D10 | ATSTLAS | 82 |
| 73C8 | AASTLAS | 80 |
| 74F4 | GASTLAS | 71 |
| N/A | X106-X107-S-T-L-A-S | 260 |

As used herein, X98 is D, G, Q, 5, or Y, X99 is A or T, X100 is A or 5, X101 is D, G, Q, or Y, X102 is A or T, X103 is A or T, X104 is R or 5, X105 is A or 5, X106 is A or G, and X107 is A or T.

TABLE 16
Consensus Sequences of Light Chain CDR3 in Exemplary Antigen Binding Molecules
(Kabat and IMGT Numbering Scheme)

| Clone | LCDR3 | SEQ ID NO: |
|---|---|---|
| 33E8 | QQGATRINIDNA | 86 |
| 53G7 | QQGASRINIDNA | 87 |
| N/A | Q-Q-G-A-X108-R-I-N-I-D-N-A | 261 |
| 74A9 | QTYYLTTTTNA | 88 |
| 38D10 | QTYYLTSTINA | 89 |
| N/A | Q-T-Y-Y-L-T-X109-T-X110-N-A | 262 |
| 55H3/76D6 | QQGWTVSSLDNA | 90 |
| 56E5 | QQGWTVSSLDNA | 90 |
| 57B10 | QQGWTVSSLDNA | 90 |
| 57F11 | QQGWTVSSLENA | 91 |
| 62E6 | QQGWTVSSLDNA | 90 |
| 69D10 | QQGWTVSSLDNA | 90 |
| 73C8 | QQGWTVSSLDNA | 90 |
| 74F4 | QQGWTVSSLDNA | 90 |
| N/A | Q-Q-G-W-T-V-S-S-L-X111-N-A | 263 |

As used herein, X108 is S or T, X119 is S or T, X110 is I or T, and X111 is D or E.

In some embodiments, the antibody, or the antigen binding fragment thereof, comprises: (1) a heavy chain variable region (HCVR) having an amino acid sequence that is about 80%, 85%, 90%, 95%, 96%, 97%, 98%, 99% to about 100% identical to an amino acid sequence selected from the HCVR sequence listed in Tables 17 and 18; and (2) a light chain variable region (LCVR) having an amino acid sequence that is about 80%, 85%, 90%, 95%, 96%, 97%, 98%, 99% to about 100% identical to an amino acid sequence selected from the LCVR sequence listed in Tables 19 and 20, wherein the antibody, or the antigen binding fragment thereof, binds specifically to human PD-L1. Exemplary HCVR- and LCVR amino acid sequences corresponding to the exemplary anti-human PD-L1 monoclonal antibodies disclosed in the present invention are shown in Tables 17-20.

TABLE 17
Amino Acid Sequences of HCVRs of Exemplary Antigen Binding Molecules

| Clone | HCVR Sequences | SEQ ID NOS |
|---|---|---|
| 27A2 | QEQLEESGGGLVQPEGSLTLTCTASGFSFSSNHYMSWVRQAPGKGLEWIGSIGVGSGITDYASWAKGR | 92 |
| | FTISKASSTTLTLQMTTLTAADTAIYFCARWTSGGGGFGLWGPGTLVTVSS | |
| 26G2 | QEQLEESGGGLVQPEGSLTLTCTASGFSFSSNHYMCWVRQAPGKGLEWIGCIGVGSGITDYASWAKGR | 93 |
| | FTISKASSTTLTLQMTTLTAADTAIYFCARWTSGGGGFGLWGPGTLVTVSS | |
| 28A12 | QEQLVESGGGLVQPEGSLTLTCTASGFSFSSAYYMCWVRQAPGKGLEWVGCIGVGSGITDYASWAKGR | 94 |
| | FTISKTSSTTVTLQMTTLTAADTAIYFCARWTSGGGGFGLWGPGTLVTVSS | |
| 72G5 | QEQLVESGGGLVQPEGSLTLTCTASGFSFSASYYMCWVRQAPGKGLEWIGCIGIGSGITDYANWAEGR | 95 |
| | FTISKTSTTVTLQMTTLTAADTAIYFCARWTSGGGGFGLWGPGTLVTVSS | |
| 67B6 | QEQLVESGGGLVQPEGSLTLTCTASEFSFSSNYYMCWVRQAPGKGLEWIGCIGIGSYITDYASWAKGR | 96 |
| | FTISKTSSTTVTLQMTTLTAADTAIYFCARWTSGGGGFGLWGPGTLVTVSS | |
| 35C9 | QEQLVESGGGLVQPEGSLTLTCTASAFSFNNNHYMCWVRQAPGKGLEWIGCIGIGSGISDYANWAKGR | 97 |
| | FTISKTSSTTVTLQMTTLTAADTAIYFCARWTSGGGGFGLWGPGTLVTVSS | |
| 65B6 | QEQLVESGGGLVQPEGSLTLTCTASEFSFGANYYMCWVRQAPGKGLEWIGCIGIGSGITDYASWAKGR | 98 |
| | FTISKTSSTTVTLQMTTLTAADTAIYFCARWTSGGGGFGLWGPGTLVTVSS | |
| 56H3 | QEQLVESGGGLVQPEGSLTLTCTASEFSFSNNYYMSWVRQAPGKGLEWLGSIGIGSGITDYASWAKGR | 99 |
| | FTISKASSTTVTLQMTTLTAADTAIYFCARWTSGGGGFGLWGPGTLVTVSS | |
| 39C10 | QEQLVESGGGLVQPEGSLTLTCTASEFSFSNNYYMCWVRQAPGKGLEWLGCIGIGSGITDYASWAKGR | 100 |
| | FTISKASSTTVTLQMTTLTAADTAIYFCARWTSGGGGFGLWGPGTLVTVSS | |
| 27C10 | QEQLVESGGGLVQPEGSLTLTCTASEFSFSSNYYMCWVRQAPGKGLEWIGCIGIGSGISDYASWAKGR | 101 |
| | FTISKASSTTVTLQMTTLTAADTAIYFCARWTSGGGGFGLWGPGTLVTVSS | |
| 33H7 | QEQLVESGGGLVQPEGSLTVTCTASGFDLSRYFYMSWVRQAPGKGLEWIASISTGSDITDYASWAKGR | 102 |
| | FTISKTSSTTVTLQMTTLTAADTAIYFCARWTSGGGGFGLWGPGTLVTVSS | |
| 30A6 | QEQLEESGGGLVQPEGSLTLTCTASGFDFSSAYWICWVRQAPGKGLEWIGCISTGSAITDYASWAKGR | 103 |
| | FTISKTSSTTVTLQMTTLTAADTAIYFCARWTSGGGGFGLWGPGTLVTVSS | |
| 32E2 | QEQLEESGGDLVKPEGSLTLTCTASGFSFSSSYYMSWVRQAPGMGLEWIASISGGVTDNAYYASWAKG | 104 |
| | RFTVSKTSSTTVTLQLTSLTAADTATYFCARDSSSGYFFLLWGPGTLVTVSS | |
| 28B3 | QSVEESGGRLVTPGTPLTLSCTASGFSLSSYYMIWVRQAPGKGLEYIGYVYTGSGNTWYASWAKGRFA | 105 |
| | ISKASTTVDLMITSPTTEDTATYFCASGADGVYDWGWDIWGPGTLVTVSL | |
| 33E8 | QSLEESGGDLVKPGASLTLTCTASGFYFSSGYDMSWVRQAPGKGLEWIASIFTTSGSTWYANWAKGRF | 106 |
| | TISKTSSTTVTLQMTSLTDADTATYFCTKDGVGSFYMNLWGPGTLVTVSS | |
| 53G7 | QSLEESGGDLVKPGASLTLTCTASGFYFSSGYDMCWVRQAPGKGLEWIACIFTGSGSTWYANWAKGRF | 107 |
| | TISKTSSTTVTLQMTSLTDADTATYFCTRDGAGSFYMNLWGPGTLVTVSS | |
| 74A9 | QSVEESGGRLVTPGTPLTLTCAVSGFSLSTYALGWVRQAPGKGLEWIGSISIGGATYYASWAKGRFTI | 108 |
| | SKTSTTVDLKIASPTTEDTATYFCARNVDSIYLDAFHTWGPGTLVTVSL | |
| 38D10 | QSVEESGGRLVTPGTPLTLTCTVSGIDLSTYAMGWVRQAPGKGLEWIGSISIGGATYFATWAKGRFTI | 109 |
| | SKGSTTVDLKIASPTTEDTATYFCARNVDIIYLNAFDIWGPGTLVTVSL | |
| 55H3/ | QEQLEESGGGLVQPEGSLTLTCTVSGFSFSGDYYMSWVRQAPGKGLEWVASIYTGSLNSYYATWAKGR | 110 |
| 76D6 | FTISLTSSTTVTLQMTSLTAADTATYFCARRNKNADYGHFDLWGPGTLVTVSS | |
| 56E5 | QEQLEESGGGLVQPEGSLTLTCTASGFSFSGDYYMSWVRQAPGKGLEWIASIYTGSLNTYYATWAKGR | 111 |
| | FTISLTSSTTVTLQMTSLTAADTATYFCARRHKNADYGHFDLWGPGTLVTVSS | |
| 57B10 | QEQLEESGGGLVQPEGSLTLTCTASGFSFSDDYYMSWVRQAPGKGLEWIASIYTGSLNTYYATWAKGR | 112 |
| | FTISLTSSTTVTLQMTSLTAADTATYFCARRNKNADWGHFDLWGPGTLVTVSS | |
| 57F11 | QEHLEESGGGLVQPEGALTLTCTASGFSFSGDYYMSWVRQAPGKGLEWIASIYTGSLNTYYATWAKGR | 113 |
| | FTISIASSTTVTLQVTSLTAADTATYFCARRTKNGDYGYFDLWGPGTLVTVSS | |
| 62E6 | QEQLEESGGDLVKPEGSLTLTCTASGFSFSGDYYMSWVRQAPGKGLEWIASIYTGSLNIYYATWAKGR | 114 |
| | FTISLTSSTTVTLQMTSLTAADTATYFCARRNKNADYGHFDLWGPGTLVTVSS | |
| 69D10 | QEQLEESGGGLVQPEGSLTLTCTASGFSFSGDWYMSWVRQAPGKGLEWIASIYSGSLNTYYATWAKGR | 115 |
| | FTVSLTSSTTVTLQMTSLTAADTATYFCARRNKNADYGHFDLWGPGTLVTVSS | |
| 73C8 | QEQLEESGGGLVQPEGSLTLTCTVSGFSFSGDYYMSWVRQAPGKGLEWVASIYTGSLNTYYATWAKGR | 116 |
| | FTISLTSSTTVTLQMTSLTAADTATYFCARRNKNADYGHFDLWGPGTLVTVSS | |
| 74F4 | QEQLEESGGGLVQPEGSLTLTCTASGFSFSGDYYMSWVRQAPGKGLEWIASIYTGSLNTYYATWAKGR | 117 |
| | FTISLTSSTTVTLQMTSLTAADTATYFCARRNKNADYGHFDLWGPGTLVTVSS | |

TABLE 18
Amino Acid Sequences of Pyroglutamylated HCVRs of Exemplary Antigen Binding Molecules

| Clone | Pyroglutamylated HCVR Sequences | SEQ ID NOS |
|---|---|---|
| 27A2 | pEEQLEESGGGLVQPEGSLTLTCTASGFSFSSNHYMSWVRQAPGKGLEWIGSIGVGSGITDYASWAKG | 264 |
| | RFTISKASSTTLTLQMTTLTAADTAIYFCARWTSGGGGFGLWGPGTLVTVSS | |
| 26G2 | PEEQLEESGGGLVQPEGSLTLTCTASGFSFSSNHYMCWVRQAPGKGLEWIGCIGVGSGITDYASWAKG | 265 |
| | RFTISKASSTTLTLQMTTLTAADTAIYFCARWTSGGGGFGLWGPGTLVTVSS | |
| 28A12 | PEEQLVESGGGLVQPEGSLTLTCTASGFSFSSAYYMCWVRQAPGKGLEWVGCIGVGSGITDYASWAKG | 266 |
| | RFTISKTSSTTVTLQMTTLTAADTAIYFCARWTSGGGGFGLWGPGTLVTVSS | |
| 72G5 | PEEQLVESGGGLVQPEGSLTLTCTASGFSFSASYYMCWVRQAPGKGLEWIGCIGIGSGITDYANWAEG | 267 |
| | RFTISKTSTTVTLQMTTLTAADTAIYFCARWTSGGGGFGLWGPGTLVTVSS | |
| 67B6 | PEEQLVESGGGLVQPEGSLTLTCTASEFSFSSNYYMCWVRQAPGKGLEWIGCIGIGSYITDYASWAKG | 268 |
| | RFTISKTSSTTVTLQMTTLTAADTAIYFCARWTSGGGGFGLWGPGTLVTVSS | |
| 35C9 | PEEQLVESGGGLVQPEGSLTLTCTASAFSFNNNHYMCWVRQAPGKGLEWIGCIGIGSGISDYANWAKG | 269 |
| | RFTISKTSSTTVTLQMTTLTAADTAIYFCARWTSGGGGFGLWGPGTLVTVSS | |
| 65B6 | PEEQLVESGGGLVQPEGSLTLTCTASEFSFGANYYMCWVRQAPGKGLEWIGCIGIGSGITDYASWAKG | 270 |
| | RFTISKTSSTTVTLQMTTLTAADTAIYFCARWTSGGGGFGLWGPGTLVTVSS | |
| 56H3 | pEEQLVESGGGLVQPEGSLTLTCTASEFSFSNNYYMSWVRQAPGKGLEWLGSIGIGSGITDYASWAKG | 271 |
| | RFTISKASSTTVTLQMTTLTAADTAIYFCARWTSGGGGFGLWGPGTLVTVSS | |
| 39C10 | PEEQLVESGGGLVQPEGSLTLTCTASEFSFSNNYYMCWVRQAPGKGLEWLGCIGIGSGITDYASWAKG | 272 |
| | RFTISKASSTTVTLQMTTLTAADTAIYFCARWTSGGGGFGLWGPGTLVTVSS | |
| 27C10 | PEEQLVESGGGLVQPEGSLTLTCTASEFSFSSNYYMCWVRQAPGKGLEWIGCIGIGSGISDYASWAKG | 273 |
| | RFTISKASSTTVTLQMTTLTAADTAIYFCARWTSGGGGFGLWGPGTLVTVSS | |
| 33H7 | PEEQLVESGGGLVQPEGSLTVTCTASGFDLSRYFYMSWVRQAPGKGLEWIASISTGSDITDYASWAKG | 274 |
| | RFTISKTSSTTVTLQMTTLTAADTAIYFCARWTSGGGGFGLWGPGTLVTVSS | |
| 30A6 | PEEQLEESGGGLVQPEGSLTLTCTASGFDFSSAYWICWVRQAPGKGLEWIGCISTGSAITDYASWAKG | 275 |
| | RFTISKTSSTTVTLQMTTLTAADTAIYFCARWTSGGGGFGLWGPGTLVTVSS | |
| 32E2 | PEEQLEESGGDLVKPEGSLTLTCTASGFSFSSSYYMSWVRQAPGMGLEWIASISGGVTDNAYYASWAK | 276 |
| | GRFTVSKTSSTTVTLQLTSLTAADTATYFCARDSSSGYFFLLWGPGTLVTVSS | |
| 28B3 | pESVEESGGRLVTPGTPLTLSCTASGFSLSSYYMIWVRQAPGKGLEYIGYVYTGSGNTWYASWAKGRF | 277 |
| | AISKASTTVDLMITSPTTEDTATYFCASGADGVYDWGWDIWGPGTLVTVSL | |
| 33E8 | pESLEESGGDLVKPGASLTLTCTASGFYFSSGYDMSWVRQAPGKGLEWIASIFTTSGSTWYANWAKGR | 278 |
| | FTISKTSSTTVTLQMTSLTDADTATYFCTKDGVGSFYMNLWGPGTLVTVSS | |
| 53G7 | pESLEESGGDLVKPGASLTLTCTASGFYFSSGYDMCWVRQAPGKGLEWIACIFTGSGSTWYANWAKGR | 279 |
| | FTISKTSSTTVTLQMTSLTDADTATYFCTRDGAGSFYMNLWGPGTLVTVSS | |
| 74A9 | pESVEESGGRLVTPGTPLTLTCAVSGFSLSTYALGWVRQAPGKGLEWIGSISIGGATYYASWAKGRFT | 280 |
| | ISKTSTTVDLKIASPTTEDTATYFCARNVDSIYLDAFHTWGPGTLVTVSL | |
| 38D10 | pESVEESGGRLVTPGTPLTLTCTVSGIDLSTYAMGWVRQAPGKGLEWIGSISIGGATYFATWAKGRFT | 281 |
| | ISKGSTTVDLKIASPTTEDTATYFCARNVDIIYLNAFDIWGPGTLVTVSL | |
| 55H3/ | pEEQLEESGGGLVQPEGSLTLTCTVSGFSFSGDYYMSWVRQAPGKGLEWVASIYTGSLNSYYATWAKG | 282 |
| 76D6 | RFTISLTSSTTVTLQMTSLTAADTATYFCARRNKNADYGHFDLWGPGTLVTVSS | |
| 56E5 | PEEQLEESGGGLVQPEGSLTLTCTASGFSFSGDYYMSWVRQAPGKGLEWIASIYTGSLNTYYATWAKG | 283 |
| | RFTISLTSSTTVTLQMTSLTAADTATYFCARRHKNADYGHFDLWGPGTLVTVSS | |
| 57B10 | pEEQLEESGGGLVQPEGSLTLTCTASGFSFSDDYYMSWVRQAPGKGLEWIASIYTGSLNTYYATWAKG | 284 |
| | RFTISLTSSTTVTLQMTSLTAADTATYFCARRNKNADWGHFDLWGPGTLVTVSS | |
| 57F11 | pEEHLEESGGGLVQPEGALTLTCTASGFSFSGDYYMSWVRQAPGKGLEWIASIYTGSLNTYYATWAKG | 285 |
| | RFTISIASSTTVTLQVTSLTAADTATYFCARRTKNGDYGYFDLWGPGTLVTVSS | |
| 62E6 | PEEQLEESGGDLVKPEGSLTLTCTASGFSFSGDYYMSWVRQAPGKGLEWIASIYTGSLNIYYATWAKG | 286 |
| | RFTISLTSSTTVTLQMTSLTAADTATYFCARRNKNADYGHFDLWGPGTLVTVSS | |
| 69D10 | PEEQLEESGGGLVQPEGSLTLTCTASGFSFSGDWYMSWVRQAPGKGLEWIASIYSGSLNTYYATWAKG | 287 |
| | RFTVSLTSSTTVTLQMTSLTAADTATYFCARRNKNADYGHFDLWGPGTLVTVSS | |
| 73C8 | PEEQLEESGGGLVQPEGSLTLTCTVSGFSFSGDYYMSWVRQAPGKGLEWVASIYTGSLNTYYATWAKG | 288 |
| | RFTISLTSSTTVTLQMTSLTAADTATYFCARRNKNADYGHFDLWGPGTLVTVSS | |
| 74F4 | PEEQLEESGGGLVQPEGSLTLTCTASGFSFSGDYYMSWVRQAPGKGLEWIASIYTGSLNTYYATWAKG | 289 |
| | RFTISLTSSTTVTLQMTSLTAADTATYFCARRNKNADYGHFDLWGPGTLVTVSS | |

TABLE 19
Amino Acid Sequences of LCVRs of Exemplary Antigen Binding Molecules

| Clone | LCVR Sequences | SEQ ID NOS |
|---|---|---|
| 27A2 | QVLTQTSSPVSAAVGGTVTINCQSSQTIYSDYLFWYQQTPGQPPKLLIYQTSTLASGVPSRFKGSGSGTEFTLT | 118 |
| | ISEIQCDDAATYYCQGYYSGYIWTFGGGTEVVVR | |
| 26G2 | QVLTQTSSPVSAAVGGTVTINCQSSQTIYSDYLFWYQQTPGQPPKLLIYQTSTLASGVPSRFKGSGSGTEFTLT | 119 |
| | ISEIQCDDAATYYCQGYYSGYIWTFGGGTEVVVR | |
| 28A12 | QVLTQTASPVSAAVGGTVTISCQSSQSVYSNYLSWYQQKPGQPPKLLIYYASTLASGVPSRFKGSGSGTQFTLT | 120 |
| | ISGVQCDDAATYYCQGYYSGYIWTFGGGTEVVVK | |
| 72G5 | QVLTQTASPVSAAVGSTVTINCQSSQSVYSNYLAWYQQKPGQPPNLLIYGASTLASGVPSRFKGSGSGTQFTLT | 121 |
| | ISDLQCDDAATYYCQGYYSGYIWTFGGGTEVVVK | |
| 67B6 | QVLTQTSSPVSAAVGGTVTINCQSSQTIYSDYLSWYQQKPGQPPKLLIYQTSTLASGVPSRFKGSGSGTEFTLT | 122 |
| | IREIQCDDAATYYCQGYYSGYIWTFGGGTEVVVK | |
| 35C9 | QVLTQTASPVSAAVGSTVTINCQSSQNIYSDYLSWYQQKPGQPPKLLIYGASTLASGVPSRFKGNGSGTQFTLT | 123 |
| | ISDLQCDDAATYYCQGYYSGYIWTFGGGTEVVVK | |
| 65B6 | AVLTQTPSPVSAAVGGTVTINCQSSQTIYSDYLSWYQQKPGQPPKLLIYQTSTLASGVPSRFKGSGSGTEFTLT | 124 |
| | ISEIQCDDAATYYCQGYYSGYIWTFGGGTEVVVK | |
| 56H3 | QVLTQTSSPVSAAVGGTVTINCQSSQTIYSDYLFWYQQKPGQPPKLLIYQTSTLASGVPSRFSGSGSGTQFTLT | 125 |
| | ISGVQCDDAATYYCQGYYSGYIWTFGGGTEVVVN | |
| 39C10 | QVLTQTSSPVSAAVGGTVTINCQSSQTIYSDYLFWYQQKPGQPPKLLIYQTSTLASGVPSRFSGSGSGTQFTLT | 126 |
| | ISGVQCDDAATYYCQGYYSGYIWTFGGGTEVVVN | |
| 27C10 | QVLTQTSSPVSAAVGGTVTISCQSSQTIYSDYLFWYQQKPGQPPKLLIYQTSTLASGVPSRFKGSGSGTEFTLT | 127 |
| | ISEMQCDDAATYYCQGYYSGYIWTFGGGTEVVVK | |
| 33H7 | QVLTQTASPVSAAVGGTVTLNCQSSQSIYNNYLSWYQQRAGQPPKLLIYSTATLASGVPSRFKGSGSGTQFTLT | 128 |
| | ISEIQCEDAATYYCQGYYSGYIWTFGGGTEVVVK | |
| 30A6 | QVLTQTASPVSAAVGGTVTINCQSSQSVYSNYLCWYQQKPGQPPKLLMYDASTLASGVPSRFKGSGSGTQFTLT | 129 |
| | ISGVQCDDAATYYCQGYYSGYIWTFGGGTEVVVK | |
| 32E2 | LVMTQTPSSVSAAVGGTVTINCQASQNIYSNLAWYQQKPGQRPKLLIFGASNLRSGVPSRFKGSGSGTEYTLTI | 130 |
| | SDLECADAATYYCQEGYSIGNVDNPFGGGTEVVVK | |
| 28B3 | DIVMTQTPASVEAAVGDTVTIKCQASQSIYSLLAWYQQKPGQRPKLLIAGASNLESGVPSRFSGSGSGTEYTLT | 131 |
| | ISALECADAATYYCQNNYDSGRIYGLAFGGGTEVVVK | |
| 33E8 | LVMTQTPASVEVAVGGTVTIKCQASQTIGSNLAWYQQRPGQPPKLLIYKAFTLASGVSSRFKGSGSGTQFTLTI | 132 |
| | SGVECADAATYYCQQGATRINIDNAFGGGTEVVVK | |
| 53G7 | LVMTQTPASVEVPVGGTVTIKCQASGTIGSNLAWYQQKPGQPPKLLIYKTFTLASGVSSRFKGSGSGTEFTLTI | 133 |
| | SGVECADAATYYCQQGASRINIDNAFGGGTEVVVK | |
| 74A9 | DIVMTQTPASVEAAVGGTVTIKCQASQNIYNNLAWYQQKPGQPPKLLIYRASTLASGVPSRFKGSGSGTEYTLT | 134 |
| | ISDLECDDAATYYCQTYYLTTTTNAFGGGTEVVVK | |
| 38D10 | DIVMTQTPASVEAAVGGTVTIKCQASQNIYNNLAWYQQKPGQPPKLLIYSSSTLASGVSSRFKGSGSGTEYTLT | 135 |
| | ISDLECADAATYYCQTYYLTSTINAFGGGTEVVVK | |
| 55H3/ | YDMTQTPASVEVAVGGTVTIKCQASESINDRLAWYQQKPGQPPKLLIYAASTLASGVPSRFRGSGSGTDETLTI | 136 |
| 76D6 | SDLECADAATYYCQQGWTVSSLDNAFGGGTEVVAM | |
| 56E5 | YDMTQTPGSVEVAVGGTVTIKCQASQSINDRLAWYQQTPGQPPKLLIYGASTLASGVPSRFKGSGSGTEFTLTI | 137 |
| | SDLECADAATYYCQQGWTVSSLDNAFGGGTEVVAK | |
| 57B10 | YDMTQTPASVEVAVGGTVTIKCQASQSISTALAWYQQKPGQPPKLLIYAASTLASGVPSRFRGSGSGTDFTLTI | 138 |
| | SDLECADAATYYCQQGWTVSSLDNAFGGGTEVVAM | |
| 57F11 | YDMTQTPASVEVAVGGTVTIKCQASQSINDRLAWYQQKPGQPPKLLIYAASTLASGVPSRFKGSGSGTEFTLTI | 139 |
| | SDLECADAATYYCQQGWTVSSLENAFGGGTEVVAK | |
| 62E6 | YDMTQTPASVEVAVGGTVTIKCQASQSINDRLAWYQQKPRQPPKLLIYGASTLASGVPSRFKGSGSGTEFTLTI | 140 |
| | SDLECVDAATYYCQQGWTVSSLDNAFGGGTEVVAK | |
| 69D10 | YDMTQTPASVEVAVGGTVTIKCQASQSIGNALAWYQQKPGQPPKLLIYATSTLASGVPSRFKGSGSGTEFTLTI | 141 |
| | SDLECGDAATYYCQQGWTVSSLDNAFGGGTEVVAK | |
| 73C8 | YDMTQTPASVEVAVGGTVTIKCQASQSINDRLAWYQQKPGQPPKLLIYAASTLASGVPSRFRGSGSGTDFTLTI | 142 |
| | SDLECADAATYYCQQGWTVSSLDNAFGGGTEVVAM | |
| 74F4 | YDMTQTPASVEVAVGGTVTIKCQASQSINDRLAWYQQSPGQPPKLLIYGASTLASGVPSRFKGSGSGTDFTLTI | 143 |
| | SDLECADAATYYCQQGWTVSSLDNAFGGGTEVVAK | |

TABLE 20
Amino Acid Sequences of Pyroglutamylated LCVRs of Exemplary Antigen Binding Molecules

| Clone | Pyroglutamylated LCVR Sequences | SEQ ID Nos |
|---|---|---|
| 27A2 | pEVLTQTSSPVSAAVGGTVTINCQSSQTIYSDYLFWYQQTPGQPPKLLIYQTSTLASGVPSRFKGSGSGTEFTL | 290 |
| | TISEIQCDDAATYYCQGYYSGYIWTFGGGTEVVVR | |
| 26G2 | pEVLTQTSSPVSAAVGGTVTINCQSSQTIYSDYLFWYQQTPGQPPKLLIYQTSTLASGVPSRFKGSGSGTEFTL | 291 |
| | TISEIQCDDAATYYCQGYYSGYIWTFGGGTEVVVR | |
| 28A12 | pEVLTQTASPVSAAVGGTVTISCQSSQSVYSNYLSWYQQKPGQPPKLLIYYASTLASGVPSRFKGSGSGTQFTL | 292 |
| | TISGVQCDDAATYYCQGYYSGYIWTFGGGTEVVVK | |
| 72G5 | pEVLTQTASPVSAAVGSTVTINCQSSQSVYSNYLAWYQQKPGQPPNLLIYGASTLASGVPSRFKGSGSGTQFTL | 293 |
| | TISDLQCDDAATYYCQGYYSGYIWTFGGGTEVVVK | |
| 67B6 | pEVLTQTSSPVSAAVGGTVTINCQSSQTIYSDYLSWYQQKPGQPPKLLIYQTSTLASGVPSRFKGSGSGTEFTL | 294 |
| | TIREIQCDDAATYYCQGYYSGYIWTFGGGTEVVVK | |
| 35C9 | pEVLTQTASPVSAAVGSTVTINCQSSQNIYSDYLSWYQQKPGQPPKLLIYGASTLASGVPSRFKGNGSGTQFTL | 295 |
| | TISDLQCDDAATYYCQGYYSGYIWTFGGGTEVVVK | |
| 56H3 | pEVLTQTSSPVSAAVGGTVTINCQSSQTIYSDYLFWYQQKPGQPPKLLIYQTSTLASGVPSRFSGSGSGTQFTL | 296 |
| | TISGVQCDDAATYYCQGYYSGYIWTFGGGTEVVVN | |
| 39C10 | pEVLTQTSSPVSAAVGGTVTINCQSSQTIYSDYLFWYQQKPGQPPKLLIYQTSTLASGVPSRFSGSGSGTQFTL | 297 |
| | TISGVQCDDAATYYCQGYYSGYIWTFGGGTEVVVN | |
| 27C10 | pEVLTQTSSPVSAAVGGTVTISCQSSQTIYSDYLFWYQQKPGQPPKLLIYQTSTLASGVPSRFKGSGSGTEFTL | 298 |
| | TISEMQCDDAATYYCQGYYSGYIWTFGGGTEVVVK | |
| 33H7 | pEVLTQTASPVSAAVGGTVTLNCQSSQSIYNNYLSWYQQRAGQPPKLLIYSTATLASGVPSRFKGSGSGTQFTL | 299 |
| | TISEIQCEDAATYYCQGYYSGYIWTFGGGTEVVVK | |
| 30A6 | pEVLTQTASPVSAAVGGTVTINCQSSQSVYSNYLCWYQQKPGQPPKLLMYDASTLASGVPSRFKGSGSGTQFTL | 300 |
| | TISGVQCDDAATYYCQGYYSGYIWTFGGGTEVVVK | |

In certain embodiments, the antigen binding molecules of the present invention, e.g., an antibody or an antigen binding fragment thereof, are modified after translation. Examples of the posttranslational modification include cleavage of lysine at the C terminal of the heavy chain by a carboxypeptidase; modification of glutamine or glutamic acid at the N terminal of the heavy chain and the light chain to pyroglutamic acid by pyroglutamylation; glycosylation; oxidation; deamidation; and glycation, and it is known that such posttranslational modifications occur in various antibodies (See journal of Pharmaceutical Sciences, 2008, Vol. 97, p. 2426-2447, incorporated by reference in its entirety). Examples of an antigen binding molecule, e.g., an antibody or antigen binding fragment thereof which have undergone posttranslational modification include an antigen binding molecule, e.g., an antibody or antigen binding fragments thereof which have undergone pyroglutamylation at the N terminal of the heavy chain variable region and/or deletion of lysine at the C terminal of the heavy chain. The sequences of exemplary antigen binding molecules that undergo pyroglutamylation at the N-terminus is listed in Table 7. As used herein, “pE” refers to pyroglutamic acid when used to represent an amino acid in a polypeptide.

### 2. Variants of Antigen Binding Molecules

In certain embodiments, the PD-L1 antigen binding molecules of the present invention, e.g., the anti-PD-L1 antibodies, can be a monoclonal antibody, a chimeric antibody, a humanized antibody, a Fab, a (Fab)₂, a scFv or a multi-specific antibody comprising additional binding specificities described herein.

Accordingly, in certain embodiments the anti-PD-L1 antibodies described herein may be linked to an Fc comprising one or more modifications, typically to alter one or more functional properties of the antibody, such as serum half-life, complement fixation, Fc receptor binding, and/or antigen-dependent cellular cytotoxicity. Furthermore, an antibody described herein may be chemically modified (e.g., one or more chemical moieties can be attached to the antibody) or it may be modified to alter its glycosylation, to alter one or more functional properties of the antibody. More specifically, in certain embodiments, the antibodies in the present invention may include modifications in the Fc region in order to generate an Fc variant with (a) increased or decreased antibody-dependent cell-mediated cytotoxicity (ADCC), (b) increased or decreased complement mediated cytotoxicity (CDC), (c) increased or decreased affinity for C1q and/or (d) increased or decreased affinity for a Fc receptor relative to the parent Fc. Such Fc region variants will generally comprise at least one amino acid modification in the Fc region. Combining amino acid modifications is thought to be particularly desirable. For example, the variant Fc region may include two, three, four, five, etc. substitutions therein, e.g., of the specific Fc region positions identified herein.

For uses where effector function is to be avoided altogether, e.g., when antigen binding alone is sufficient to generate the desired therapeutic benefit, and effector function only leads to (or increases the risk of) undesired side effects, IgG4 antibodies or ADCC-null version of IgG1 L234F, L235E, P331S may be used, or antibodies or fragments lacking the Fc region or a substantial portion thereof can be devised, or the Fc may be mutated to eliminate glycosylation altogether (e.g., N297A). Alternatively, a hybrid construct of human IgG2 (CH1 domain and hinge region) and human IgG4 (C_{H}2 and C_{H}3 domains) may be generated that is devoid of effector function, lacking the ability to bind FcγRs (like IgG2) and activate complement (like IgG4). When using an IgG4 constant domain, it is usually preferable to include the substitution S228P which mimics the hinge sequence in IgG1 and R409K mutation which prevents Fab arm exchange and thereby stabilizes IgG4 molecules, reducing Fab-arm exchange between the therapeutic antibody and endogenous IgG4 in the patient being treated.

In certain embodiments, the anti-PD-L1 antibody or fragment(s) thereof may be modified to provide increased biological half-life. Various approaches may be employed, including e.g., those that increase the binding affinity of the Fc region for FcRn. In one embodiment, the antibody is altered within the CH1 or CL region to contain a salvage receptor binding epitope taken from two loops of a C_{H}2 domain of an Fc region of an IgG, as described in U.S. Pat. Nos. 5,869,046 and 6,121,022. The numbering of residues in the Fc region is that of the EU index of Kabat. Sequence variants disclosed herein are provided with reference to the residue number followed by the amino acid that is substituted in place of the naturally occurring amino acid, optionally preceded by the naturally occurring residue at that position. Where multiple amino acids may be present at a given position, e.g., if sequences differ between naturally occurring isotypes, or if multiple mutations may be substituted at the position, they are separated by slashes (e.g., “X/Y/Z”).

Exemplary Fc variants that increase binding to FcRn and/or improve pharmacokinetic properties include substitutions at positions 259, 308, and 434, including for example 2591, 308F, 428L, 428M, 434S, 434H, 434F, 434Y, and 434M. Other variants that increase Fc binding to FcRn include: 250E, 250Q, 428L, 428F, 250Q/428L (Hinton et al., 2004, J. Biol. Chem. 279(8): 6213-6216, Hinton et al. 2006 Journal of Immunology 176:346-356), 256A, 272A, 305A, 307A, 31 IA, 312A, 378Q, 380A, 382A, 434A (Shields et al. (2001) J. Biol. Chem., 276(9):6591-6604), 252F, 252Y, 252W, 254T, 256Q, 256E, 256D, 433R, 434F, 434Y, 252Y/254T/256E, 433K/434F/436H (Dall'Acqua et al. (2002) J. Immunol., 169:5171-5180, Dall'Acqua et al. (2006) J. Biol. Chem., 281:23514-23524, and U.S. Pat. No. 8,367,805.

Modification of certain conserved residues in IgG Fc (1253, H310, Q311, H433, N434), such as the N434A variant (Yeung et al. (2009) J. Immunol. 182:7663), have been proposed as a way to increase FcRn affinity, thus increasing the half-life of the antibody in circulation (WO 98/023289). The combination Fc variant comprising M428L and N434S has been shown to increase FcRn binding and increase serum half-life up to five-fold (Zalevsky et al. (2010) Nat. Biotechnol. 28:157). The combination Fc variant comprising T307A, E380A and N434A modifications also extends half-life of IgG1 antibodies (Petkova et al. (2006) Int. Immunol. 18:1759). In addition, combination Fc variants comprising M252Y-M428L, M428L-N434H, M428L-N434F, M428L-N434Y, M428L-N434A, M428L-N434M, and M428L-N434S variants have also been shown to extend half-life (U.S. 2006/173170). Further, a combination Fc variant comprising M252Y, S254T and T256E was reported to increase half-life-nearly 4-fold. Dall'Acqua et al. (2006) J. Biol. Chem. 281:23514.

In certain embodiments, the PD-L1 antigen binding molecule of the present invention is a bispecific antibody, comprising: a first targeting domain that binds specifically to PD-L1 and a second targeting domain that binds specifically another epitope in PD-L1 or another protein. In some embodiments, the first targeting domain includes an antigen binding fragment from any of the PD-L1 antibodies of the present invention. In certain embodiments, the first targeting domain of the bispecific antibody binds specifically to PD-L1 and the second targeting domain specifically binds to a protein expressed on a surface of an immune cell, such as a T cell, a NK cell, a NK T cell, a macrophage, a dendritic cell or a Myeloid-derived suppressor cells (MDSC). Without wishing to be bound by any theory, it is hypothesized that a bispecific antibody may bind to PD-L1 on the surface of a tumor cell and a protein expressed on a surface of an immune cell. The bispecific antibody thus facilitates the killing of the tumor cell by the immune cell.

In certain embodiments, the antigen binding molecules, e.g., anti-PD-L1 antibodies or antigen binding fragments thereof, of the present invention are chemically conjugated to one or more therapeutically active peptides and/or small molecule drugs to form an antibody drug conjugate (ADC). The peptides or small molecule drugs can be attached, for example to reduced SH groups and/or to carbohydrate side chains. Methods for making covalent or non-covalent conjugates of peptides or small molecule drugs with antibodies are known in the art and any such known method may be utilized. Without wishing to be bound by any theory, it is hypothesized that the ADC according to the present disclosure can bind to PD-L1 expressed on the surface of a tumor cell. The internalization of the antibody-antigen complex thus introduce the drug into a tumor cell, thereby the tumor cell may be killed by the drug conjugated to the PD-L1 of the present disclosure.

In some embodiments the peptide or small molecule drug is attached to the hinge region of a reduced antibody component via disulfide bond formation. Alternatively, such agents can be attached using a heterobifunctional cross-linkers, such as N-succinyl 3-(2-pyridyldithio)propionate (SPDP). General techniques for such conjugation are well-known in the art. In some embodiments, the peptide or small molecule drug is conjugated via a carbohydrate moiety in the Fc region of the antibody. The carbohydrate group can be used to increase the loading of the same agent that is bound to a thiol group, or the carbohydrate moiety can be used to bind a different therapeutic or diagnostic agent. Methods for conjugating peptide inhibitors or small molecule drugs to antibodies via antibody carbohydrate moieties is well-known to those of skill in the art. For example, in one embodiment, the method involves reacting an antibody component having an oxidized carbohydrate portion with a carrier polymer that has at least one free amine function. This reaction results in an initial Schiff base (imine) linkage, which can be stabilized by reduction to a secondary amine to form the final conjugate. Exemplary methods for conjugating small molecule drugs and peptides to antibodies are described in U.S. Patent Application Publication No. 2014/0356385.

### 3. Biological Characteristics of the Antibodies and Antigen Binding Molecules

The present invention includes antibodies and antigen binding fragments thereof that bind human and cynomolgus PD-L1.

The present invention includes PD-L1 antigen binding molecules, e.g., PD-L1 antibodies or the antigen binding fragments thereof, which are capable of specifically binding to human and cynomolgus PD-L1 expressed on a cell surface and inhibits the PD-L1 activities or functions. According to certain embodiments, the antigen binding molecules blocks the interaction between the human PD-L1 expressed on a cell surface and PD-L1 agonist, e.g., PD-1. The extent to which an PD-L1 antigen binding protein, e.g., an PD-L1 antibody or an antigen binding fragment thereof, blocks the interaction between PD-1 and PD-L1 expressed on a cell surface, can be assessed by the assays described in Example 5, or a substantially similar assay. The present invention includes antigen binding molecules, e.g., antibodies or antigen binding fragments thereof, which blocks the interaction between human PD-L1 expressed on a cell surface and an PD-1 with an IC₅₀ value of about 1 nM, about 0.75 nM, about 0.5 nM, or less, or between about 0.2 to 1.0 nM, as determined using an assay as set forth in Example 5, or a substantially similar assay.

The present invention includes PD-L1 antigen binding molecules, e.g., PD-L1 antibodies or the antigen binding fragments thereof, which are capable of competing with PD-1 to bind PD-L1 and blocking the interaction between PD-L1 and PD-1 protein. In certain embodiments, the antigen binding molecules of the present invention, e.g., a PD-L1 antibody or an antigen binding fragment thereof, competes with PD-1 to bind PD-L1 and block the interaction between PD-L1 and PD1, as determined using a competitive ELISA assay as set forth in Example 5. In certain embodiments, the present invention includes antigen binding molecules, e.g., antibodies or antigen binding fragments thereof, which competes with PD-1 to bind to PD-L1 and blocks the interaction of PD-1 and PD-L1 with an IC₅₀ value of about 10 nM, about 7.5 nM, about 5.0 nM, about 4.0 nM, about 3.0 nM, about 2.5 nM, about 2.0 nM, about 1.5 nM, about 1.0 nM or less, or between about 1.0 and 10 nM, as determined using a competition ELISA assay as set forth in Example 5, or a substantially similar assay.

The present invention includes PD-L1 antigen binding molecules, e.g., PD-L1 antibodies or the antigen binding fragments thereof, which bind to human PD-L1 or non-human primate PD-L1 specifically. In certain embodiments, the binding of the antigen binding molecules of the present invention to a PD-L1 family member other than PD-L1 or PD-L1 from certain non-human mammal, e.g., murine PD-L1, is either undetectable or weak, as determined using an assay as set forth in Example 3, or a substantially similar assay. In certain embodiments, the present invention includes antigen binding molecules, e.g., antibodies or antigen binding fragments thereof, which binds to human PD-L1 or cynomolgus PD-L1 with an EC₅₀ value of about 0.2 nM, 0.1 nM, 0.09 nM, 0.08 nM, 0.07 nM, 0.06 nM, 0.05 nM, 0.04 nM, 0.03 nM, about 0.02 nM, about 0.01 nM, about 0.009 nM, about 0.008 nM, about 0.007 nM, or less, or between about 0.007 nM and 0.2 nM, as determined using an assay as set forth In Example 3, or a substantially similar assay, while the antigen binding molecules, e.g., PD-L1 antibodies or antigen binding fragment thereof, does not show binding to a PD-L1 family member other than PD-L1 or PD-L1 from certain non-human mammal, e.g., murine PD-

The present invention includes PD-L1 antigen binding molecules, e.g., PD-L1 antibodies, which bind to human or cynomolgus PD-L1 with high affinity. In certain embodiments, the present invention includes antigen binding molecules, e.g., antibodies or antigen binding fragments thereof, which bind to human or cynomolgus PD-L1 with a K_{D} value from about 10 nM, about 5 nM, about 2 nM, about 1 nM, about 0.9 nM, about 0.8 nM, about 0.7 nM, about 0.6 nM, about 0.5 nM, about 0.4 nM, about 0.3 nM, about 0.2 nM, about 0.1 nM, about 0.09 nM, about 0.08 nM, about 0.05 nM, about 0.04 nM, about 0.03 nM, about 0.02 nM, about 0.01 nM or less, or between about 0.01 nM and 10 nM, as determined using an assay as set forth in Example 3, or a substantially similar assay.

The present invention includes PD-L1 antigen binding molecules, e.g., PD-L1 antibodies or antigen binding fragments thereof, which specifically bind to a PD-L1 expressing cell, e.g., cells with endogenous human and/or non-human primate PD-L1, e.g., cynomolgus PD-L1, expressed on the surface of human or non-human primate immune cells, as determined using an assay as set forth in Example 4, or a substantially similar assay. In certain embodiments, the present invention includes anti-PD-L1 antigen binding molecules, e.g., anti-PD-L1 antibodies or antigen binding fragments thereof, which bind to human or non-human private PD-L1 expressing cell with an EC₅₀ value of about 20 nM, about 18 nM, about 15 nM, about 10 nM, about 10 nM, about 5 nM, about 4 nM, about 3 nM, about 2 nM, about 1 nM, about 0.9 nM, about 0.8 nM, about 0.7 nM, about 0.6 nM, about 0.5 nM, about 0.4 nM, about 0.3 nM, about 0.2 nM, about 0.1 nM, or less, or between about 0.1 nM and 20 nM, as determined using an assay as set forth in Example 4, or a substantially similar assay.

The present invention includes PD-L1 antigen binding molecules, e.g., PD-L1 antibodies or antigen binding fragments thereof, which induce antibody-dependent cellular cytotoxicity (ADCC), as determined using an assay as set forth in Example 6, or a substantially similar assay. In certain embodiments, the present invention includes anti-PD-L1 antigen binding molecules, e.g., anti-PD-L1 antibodies or antigen binding fragments thereof, which induce ADCC with an EC₅₀ value of about 10 nM, about 5 nM, about 4 nM, about 3 nM, about 2 nM, about 1 nM, about 0.9 nM, about 0.8 nM, about 0.7 nM, about 0.6 nM, about 0.5 nM, about 0.4 nM, about 0.3 nM, about 0.2 nM, about 0.1 nM, or less, or between about 0.1 nM and about 10 nM, between about 0.1 nM and 2.5 nM, or between about 1.0 nM and 5.0 nM as determined using an assay as set forth in Example 6, or a substantially similar assay.

The present invention includes PD-L1 antigen binding molecules, e.g., PD-L1 antibodies or antigen binding fragments thereof, which induce internalization upon binding to a PD-L1, e.g., a human or a cynomolgus PD-L1, expressed on a cell surface, as determined using an assay as set forth in Example 7, or a substantially similar assay. In certain embodiments, the present invention includes anti-PD-L1 antigen binding molecules, e.g., anti-PD-L1 antibodies or antigen binding fragments thereof, which induce internalization of about 4%, about 5%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, or more, or between about 4%-about 40%, of the cell surface complexes formed by the binding of PD-L1 antigen binding molecules, e.g., anti-PD-L1 antibodies or antigen binding fragments thereof, to cell surface PD-L1, e.g., human or cynomolgus PD-L1, as determined using an assay as set forth in Example 7, or a substantially similar assay.

The present invention includes PD-L1 antigen binding molecules, e.g., PD-L1 antibodies or antigen binding fragments thereof, which induce cytotoxic reaction of PD-L1 expressing cells when the PD-L1 antigen binding molecules, e.g., PD-L1 antibodies or antigen binding fragments thereof, are conjugated to a cytotoxic agent, as determined using an assay as set forth in Example 8, or a substantially similar assay.

The present invention includes PD-L1 antigen binding molecules, e.g., PD-L1 antibodies or antigen binding fragments thereof, which belong to the same epitope bin or different epitope bin as to Avelumab, as determined using an assay as set forth in Example 9, or a substantially similar assay.

### 4. Species Selectivity and Species Cross-Reactivity

The present invention, according to certain embodiments, provides antigen binding molecules that bind to human PD-L1 but not to PD-L1 from other species. The present invention also includes antigen binding molecules that bind to human PD-L1 and to PD-L1 from one or more non-human species, e.g., non-human primates.

According to certain exemplary embodiments of the invention, antigen binding molecules are provided which bind to human PD-L1 and may bind or not bind, as the case may be, to one or more of mouse, rat, guinea pig, hamster, gerbil, pig, cat, dog, rabbit, goat, sheep, cow, horse, camel, cynomolgus, marmoset, rhesus or chimpanzee PD-L1. For example, in a particular exemplary embodiment of the present invention, antigen binding molecules are provided comprising an antigen binding domain that binds human PD-L1 and non-human primate, e.g., cynomolgus PD-L1.

### III. Therapeutic Use of the Anti-PD-L1 Antigen Binding Molecules

The anti-PD-L1 antigen binding molecules of the present invention, including antibodies, antigen binding fragment thereof, and multi-specific antibodies thereof, have numerous in vitro, in vivo and ex vivo utilities associated with enhancement of immune responses by blocking signaling by PD-L1 and other signaling pathways in the treatment of cancers. Without wishing to be bound by any theory, it is hypothesized that the antigen binding molecules of the present invention, e.g., anti-PD-L1 antibodies or antigen binding fragments thereof, binds to PD-L1 expressed on cell surface and inhibits the interaction thereof to a PD-1 and such a binding may have various effects, e.g., increasing immune activity as a result of blockade of the interaction between PD-1 and PD-L1. Accordingly, the antigen binding molecules of the invention (and therapeutic compositions comprising the same) are useful, inter alia, for treating any disease or disorder in which blockade of PD-1/PD-L1 interaction, e.g., stimulation and/or activation of an immune response, would be beneficial. In view of the expression of PD-L1 and the pleiotropic effects mediated by interaction between PD-1 and PD-L1, the anti-PD-L1 antigen binding molecules, e.g., antibodies or the antigen binding fragments thereof of the present invention may be used individually or in combination with a variety of active agents for treating a broad scope of diseases or disorders, including a variety of cancers.

Accordingly, the present invention provides a method of blocking the interaction between PD-1 and PD-L1, including contacting the cell with the antigen binding molecules of the present invention, e.g., anti-PD-L1 antibodies or antigen binding fragment thereof, with a cell. The blockade of the interaction between PD-1 and PD-L1 can be measured by a method as described in Example 5, or a substantially similar method. In certain embodiment, the methods of the invention block the concentration of the interaction of PD-1 and PD-L1 by at least about 10%, about 20%, about 50%, about 60%, about 70%, about 80%, about 90%, about 95%, or more, as compared to a baseline level.

In some embodiments, the antigen binding molecules, e.g., anti-PD-L1 antibodies or antigen binding fragments thereof, of the present invention are administered to cells in culture (in vitro) or to human subjects, in vivo or ex vivo, to enhance immunity in a variety of diseases. Accordingly, in one embodiment, a method for stimulating an immune response in a subject in need thereof includes administering to the subject an anti-PD-L1 antibody, antigen binding fragments thereof (e.g., anti-PD-L1 HCVRs and LCVRs) or multispecific anti-PD-L1 antibodies described herein, such that an immune response is enhanced, stimulated, up-regulated in the subject, for example, to inhibit tumor growth, stimulate anti-tumor T-cell immunity and/or stimulate antimicrobial immunity.

In one aspect, a method for enhancing an immune response (e.g., T cell response) in a subject includes the step of administering an anti-PD-L1 antibody described herein to a subject such that an immune response (e.g., T cell response) in the subject is enhanced. In some embodiments, the subject is a tumor-bearing subject and an immune response against the tumor is enhanced. A tumor may be a solid tumor or a liquid tumor, e.g., a hematological malignancy. In certain embodiments, the tumor is an immunogenic tumor. In other embodiments, a tumor is non-immunogenic. In other embodiments, the subject is pathogen-bearing subject in which an immune response against the pathogen is enhanced as a consequence of administering an anti-PD-L1 antibody described herein. The immune response includes, but is not limited to, a) reversing T cell inactivation and function; b) promoting T cell proliferation; c) enhancing NK cell function; or d) d) enhance the T-cell-mediated anti-tumor immune response.

In certain embodiments, the methods of the invention increase immune response by at least about 10%, about 20%, about 50%, about 60%, about 70%, about 80%, about 90%, about 1-fold, about 2-folds, about 4 folds, or more, as compared to a baseline level.

Preferred subjects include human patients in whom enhancement of an immune response would be desirable. The methods are particularly suitable for treating human patients having a disorder that can be treated by augmenting an immune response (e.g., the T-cell mediated immune response). The methods are particularly suitable for treatment of cancer, chronic infections and chronic inflammatory disease conditions. Preferably, the antibodies for use in the disclosed methods described herein are human or humanized antibodies.

In one embodiment, a method for inhibiting the growth of tumor cells in a subject comprises administering to the subject an anti-PD-L1 antibody described herein such that growth of the tumor is inhibited in the subject. The inhibition of tumor growth can be measured by various methods. The tumor growth can be measured using methods, e.g., as described in Talkington, A and Durrett, R, Estimating Tumor Growth Rates in vivo, Bull Math Biol., 2015 October: 77 (10): 1934-54, available at https://www.ncbi.nlm.nih.gov/pmc/articles/PMC4764475/, the entire contents of which are incorporated herein by reference. The inhibition of tumor growth can also be measured by the reduction of tumor size. In certain embodiment, the methods of the invention inhibit the tumor growth by at least about 10%, about 20%, about 50%, about 60%, about 70%, about 80%, about 90%, about 95%, or more, as compared to a baseline level.

In certain embodiment, the antigen binding molecules of the present invention, e.g., anti-PD-L1 antibodies or antigen binding fragments thereof, can be used in a method to reduce the immunosuppression in a tumor microenvironment. Such reduction can be measured by various methods. For example, the level immunosuppression in a tumor microenvironment can be measured by the presence and/or abundance of certain biomarkers in the tumor, such as PD-L1, CD39, CD73, LAG-3, IL-10, or TGF-β. The level of immunosuppression can also be measure by the ratio of CD8+ cytotoxic T cells to regulatory T (T_{reg}) cells in a tumor. In general, immunosuppression decreases the ration of CD8+ cytotoxic T cells to T_{reg}. In certain embodiments, the antigen binding molecules of the present invention, e.g., anti-PD-L1 antibodies or antigen binding fragments thereof, reduces the level of the immunosuppression in a tumor microenvironment by at least about 10%, about 20%, about 50%, about 60%, about 70%, about 80%, about 90%, about 95%, or more, as compared to a baseline level.

Also encompassed herein are methods for depleting T_{reg} cells from the tumor microenvironment of a subject with a tumor, e.g., cancerous tumor, comprising administering to the subject a therapeutically effective amount of an anti-PD-L1 antibody described herein that comprises an Fc that stimulates depletion of T_{reg} cells in the tumor microenvironment. An Fc may, for example, be an Fc with a suitable effector function or an enhanced effector function conferred by one or more activating Fc receptors.

In certain preferred embodiments, the subject has a cell proliferative disease or cancer. Blocking of PD-1/PD-L1 interaction with the antigen binding molecules, e.g., anti-PD-L1 antibodies or antigen binding fragments thereof, of the present invention can enhance the immune response to cancerous cells in the patient. Therefore, the present invention provides methods for treating a subject having cancer, comprising administering to the subject an anti-PD-L1 antigen binding molecule, e.g., an antibody or the antigen binding fragment thereof, as described herein, such that the subject is treated, e.g., such that growth of a cancerous tumor is inhibited or reduced and/or that the tumor regresses. The anti-PD-L1 antibody can be used alone to inhibit the growth of cancerous tumors. Alternatively, the anti-PD-L1 antibody can be used in conjunction with targeting one or more other active agents, e.g., other anti-cancer targets, immunogenic agents, standard cancer treatments, or other antibodies, as described below. The antigen binding molecules of the present invention may be used to treat, e.g., primary and/or metastatic tumors. The present invention also includes methods for treating residual cancer in a subject. As used herein, the term “residual cancer” means the existence or persistence of one or more cancerous cells in a subject following treatment with an anti-cancer therapy.

Accordingly, in one aspect, a method of treating cancer includes the step of administering to a subject in need thereof, a therapeutically effective amount of an anti-PD-L1 antibody as described herein. Preferably, the antibody inhibits the activity of human anti-PD-L1 and includes one or more HCVRs and LCVRs described herein. Further, the anti-PD-L1 antigen binding molecules, e.g., antibodies for use in this method may include chimeric or humanized non-human anti-PD-L1 antibodies therefrom. The efficacy of treating cancer can be measured by various methods. For example, the efficacy of treating cancer can be measured by improvements in survival, or reduction in tumor size. In certain embodiments, the methods of the invention increase the efficacy of treating a cancer by at least about 10%, about 20%, about 50%, about 60%, about 70%, about 80%, about 90%, about 95%, about 1-fold, about 2 folds, about 4 folds, or more, as compared to a baseline level. The baseline level, as used in the context of cancer treatment, refers to the efficacy using a placebo if the PD-L1 antigen binding molecule of the invention is the sole therapeutic agent, or the efficacy using a placebo or an additional therapeutic agent if the PD-L1 antigen binding molecule of the invention is used in combination with the additional therapeutic agent.

Cancers whose growth may be inhibited using the antibodies of the invention include a broad variety of cancers, especially those that are unresponsive or that have a tendency to become unresponsive to monotherapies with other antibodies or chemotherapeutic agents. Non-limiting examples of cancers for treatment include squamous cell carcinoma, small-cell lung cancer, non-small cell lung cancer, squamous non-small cell lung cancer (NSCLC), non NSCLC, glioma, gastrointestinal cancer, renal cancer (e.g., clear cell carcinoma), ovarian cancer, liver cancer, colorectal cancer, endometrial cancer, kidney cancer (e.g., renal cell carcinoma (RCC)), prostate cancer (e.g., hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally-induced cancers including those induced by asbestos, virus-related cancers (e.g., human papilloma virus (HPV)-related tumor), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CIVIL), undifferentiated AML (MO), myeloblastic leukemia (M1), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NEIL), B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukemia (T-Lbly/T-ALL), peripheral T-cell lymphoma, lymphoblastic lymphoma, post-transplantation lymphoproliferative disorder, true histiocytic lymphoma, primary central nervous system lymphoma, primary effusion lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of the central and peripheral nervous, including astrocytoma, schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) preferably of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, as well as any combinations of said cancers. The methods described herein may also be used for treatment of metastatic cancers, refractory cancers (e.g., cancers refractory to previous immunotherapy, e.g., with a blocking CTLA-4 or PD-1 antibody), and recurrent cancers.

In some embodiments, treatment of a cancer patient with an anti-PD-L1 antibody and/or other active agents according to the present invention may lead to a long-term durable response relative to the current standard of care, including long term survival of at least 1, 2, 3, 4, 5, 10 or more years and/or recurrence free survival of at least 1, 2, 3, 4, 5, or 10 or more years. In certain embodiments, treatment of a cancer patient with an anti-PD-L1 antibody and/or other active agents according to the present invention prevents recurrence of cancer or delays recurrence of cancer by, e.g., 1, 2, 3, 4, 5, or 10 or more years. The anti-PD-L1 treatment can be used as a primary or secondary line of treatment.

In some embodiments, ex vivo activation in the presence of anti-PD-L1 antibodies and expansion of antigen specific T cells and adoptive transfer of these cells into recipients may be employed to stimulate antigen-specific T cells against cancers or viral infections by increasing the frequency and activity of the adoptively transferred T cells.

Suitable routes for administering the antigen binding molecules, e.g., anti-PD-L1 antibodies or antigen binding fragment thereof, of the present invention (e.g., humanized monoclonal antibodies, multi-specific antibodies, and immunoconjugates) described herein in vivo, ex vivo or in vitro are well known in the art and can be selected by those of ordinary skill. For example, the antibody compositions can be administered by parenteral injection (e.g., intravenous or subcutaneous). Suitable dosages will depend on the age and weight of the subject and the concentration and/or formulation of the antibody composition as further described below.

The term “cell proliferative disorder” refers to a disorder characterized by abnormal proliferation of cells. A proliferative disorder does not imply any limitation with respect to the rate of cell growth, but merely indicates loss of normal controls that affect growth and cell division. Thus, in some embodiments, cells of a proliferative disorder can have the same cell division rates as normal cells but do not respond to signals that limit such growth. Within the ambit of “cell proliferative disorder” is a neoplasm, cancer or tumor.

The term “cancer” refers to any one of a variety of malignant neoplasms characterized by the proliferation of cells that have the capability to invade surrounding tissue and/or metastasize to new colonization sites, and includes carcinomas, sarcomas, adenocarcinomas, melanomas, leukemias, lymphomas, germ cell tumors and blastomas, including both solid and lymphoid cancers. Exemplary cancers that may be treated in accordance with the compositions and methods of the present invention include cancers of the brain, bladder, breast, cervix, colon, head and neck, kidney, lung, non-small cell lung, mesothelioma, ovary, prostate, stomach and uterus, leukemia, and medulloblastoma.

The term “carcinoma” refers to the malignant growth of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, pancreatic ductal adenocarcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “sarcoma” refers to a tumor made up of a substance like the embryonic connective tissue and is generally composed of closely packed cells embedded in a fibrillar or homogeneous substance. Exemplary sarcomas include, for example, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphomas (e.g., Non-Hodgkin Lymphoma), immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” refers to a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma subungal melanoma, and superficial spreading melanoma.

The term “lymphoma” refers to a group of cancers affecting hematopoietic and lymphoid tissues, which begins in lymphocytes, the blood cells that are found primarily in lymph nodes, spleen, thymus, and bone marrow. Two main types of lymphoma are non-Hodgkin's lymphoma and Hodgkin's disease. Hodgkin's disease represents approximately 15% of all diagnosed lymphomas.

This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas include, but are not limited to, cutaneous T-cell lymphoma, peripheral T-cell lymphoma, anaplastic large cell lymphoma, mycosis fungoides, and precursor T-lymphoblastic lymphoma.

The term “leukemia” refers to progressive, malignant diseases of the blood-forming organs and is generally characterized by a distorted proliferation and development of leukocytes and their precursors in the blood and bone marrow. Exemplary leukemias include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

Additional cancers include, for example, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, thyroid cancer, neuroblastoma, esophageal cancer, genitourinary tract cancer, malignant hypercalcemia, cervical cancer, endometrial cancer, and adrenal cortical cancer

### IV. Combination Therapies

In another aspect, the present invention provides therapeutic compositions and combination therapies for enhancing antigen-specific T cell responses, reducing immunosuppression, and/or reducing tumor growth in a subject. The present invention includes compositions and therapeutic formulations comprising any of the exemplary antigen binding molecules, e.g., herein in combination with one or more additional therapeutical agents, and methods of treatment comprising administering such combinations to subjects in need thereof. The term “additional therapeutic agent,” as used herein, refers to any agents which can be used to treat a disease or disorder, and any method of treatment for certain disease or disorder. For example, radiotherapy and surgery are deemed as “additional therapeutic agent” when they are used in combination with the antigen binding molecules, e.g., anti-PD-L1 antibodies or antigen binding fragment thereof, of the invention.

In certain embodiments, the additional therapeutic agent may be an agent that blocks the interaction between PD-1 and PD-L1 that is different to the antigen binding molecule, e.g., anti-PD-L1 antibodies or antigen binding fragment thereof, of the present invention. Exemplary blocking agents for PD1/PD-L1 interaction include, but are not limited to pembrolizumab, nivolumab, atezolizumab, avelumab, durvalumab, BMS-936559, sulfamonomethoxine 1, and sulfamethizole 2.

In some embodiments, the anti-PD-L1 antigen binding molecule, e.g., an anti-PD-L1 antibody or antigen binding fragment thereof, of the present invention is co-administered with one or more additional therapeutical agents in amount(s) effective in stimulating an immune response and/or apoptosis so as to further enhance, stimulate or upregulate an immune response and/or apoptosis in a subject. In addition, the one or more additional therapeutically active agents are administered prior to or subsequent to treatment with the anti-PD-L1 antibody.

In certain embodiments, the anti-PD-L1 antibodies described herein are administered in combination with or concurrently combined with one or other more other active agents, such as anti-cancer antibodies or polypeptides, chemotherapeutic agents, and radiotoxic agents. In other embodiments, the anti-PD-L1 antibodies described herein are administered in combination with or concurrently combined with a standard cancer treatment, such as surgery or radiation.

Co-administration of the anti-PD-L1 antibodies with these active agents or treatment modalities may address clinical deficiencies with regard to drug resistance, changes in the antigenicity of the tumor cells that render them unreactive with the antibody, and toxicities (by administering lower doses of one or more agents). PD-L1 blockade is particularly well suited for use when combined with otherwise refractory chemotherapeutic regimes. In these instances, it may be possible to achieve enhanced efficacy, but to reduce the dose of chemotherapeutic reagent administered (Mokyr et al. (1998) Cancer Research 58: 5301-5304). The rationale for PD-L1 blockade with radiation or chemotherapy is predicated on promoting cell death as a consequence of the cytotoxic action of radiation and most chemotherapeutic compounds, which can further result in increased levels of tumor antigen in the antigen presentation pathway. Other combination therapies that may act additively or synergistically with PD-L1 inhibition through cell death are surgery and hormone deprivation or inhibition. Each of these protocols further creates a source of tumor antigen in the host.

In some embodiments the anti-PD-L1 antibodies described herein are linked to another active agent in the form of an immuno-complex, immunoconjugate, or fusion protein. Alternatively, the anti-PD-L1 antibodies can be administered separate from the other active agent. In this case, the anti-PD-L1 antibodies and other antagonists can be administered before, after or concurrently with the other active agent or they may be co-administered with other known therapies, e.g., other anti-cancer agents, radiation etc. Accordingly, the present invention provides compositions and methods for providing two or more anti-cancer agents operating additively or synergistically via different mechanisms to beneficially provide both cytotoxic and immunoprotective effects in human cancer cells.

For example, in some embodiments, the anti-PD-L1 antibodies described herein may be combined with an anti-cancer agent, such an alkylating agent; an anthracycline antibiotic; an anti-metabolite; a detoxifying agent; an interferon; a polyclonal or monoclonal antibody; an EGFR inhibitor; a HER2 inhibitor; a histone deacetylase inhibitor; a hormone; a mitotic inhibitor; a phosphatidylinositol-3-kinase (PI3K) inhibitor; an Akt inhibitor; a mammalian target of rapamycin (mTOR) inhibitor; a proteasomal inhibitor; a poly(ADP-ribose) polymerase (PARP) inhibitor; a Ras/MAPK pathway inhibitor; a centrosome declustering agent; a multi-kinase inhibitor; a serine/threonine kinase inhibitor; a tyrosine kinase inhibitor; a VEGF/VEGFR inhibitor; a taxane or taxane derivative, an aromatase inhibitor, an anthracycline, a microtubule targeting drug, a topoisomerase poison drug, an inhibitor of a molecular target or enzyme (e.g., a kinase or a protein methyltransferase), a cytidine analogue or combination thereof.

Exemplary alkylating agents include, but are not limited to, cyclophosphamide (Cytoxan; Neosar); chlorambucil (Leukeran); melphalan (Alkeran); carmustine (BiCNU); busulfan (Busulfex); lomustine (CeeNU); dacarbazine (DTIC-Dome); oxaliplatin (Eloxatin); carmustine (Gliadel); ifosfamide (Ifex); mechlorethamine (Mustargen); busulfan (Myleran); carboplatin (Paraplatin); cisplatin (CDDP; Platinol); temozolomide (Temodar); thiotepa (Thioplex); bendamustine (Treanda); or streptozocin (Zanosar).

Exemplary anthracycline antibiotics include, but are not limited to, doxorubicin (Adriamycin); doxorubicin liposomal (Doxil); mitoxantrone (Novantrone); bleomycin (Blenoxane); daunorubicin (Cerubidine); daunorubicin liposomal (DaunoXome); dactinomycin (Cosmegen); epirubicin (Ellence); idarubicin (Idamycin); plicamycin (Mithracin); mitomycin (Mutamycin); pentostatin (Nipent); or varubicin (Valstar).

Exemplary anti-metabolites include, but are not limited to, fluorouracil (Adrucil); capecitabine (Xeloda); hydroxyurea (Hydrea); mercaptopurine (Purinethol); pemetrexed (Alimta); fludarabine (Fludara); nelarabine (Arranon); cladribine (Cladribine Novaplus); clofarabine (Clolar); cytarabine (Cytosar-U); decitabine (Dacogen); cytarabine liposomal (DepoCyt); hydroxyurea (Droxia); pralatrexate (Folotyn); floxuridine (FUDR); gemcitabine (Gemzar); cladribine (Leustatin); fludarabine (Oforta); methotrexate (MTX; Rheumatrex); methotrexate (Trexall); thioguanine (Tabloid); TS-1 or cytarabine (Tarabine PFS).

Exemplary detoxifying agents include, but are not limited to, amifostine (Ethyol) or mesna (Mesnex).

Exemplary interferons include, but are not limited to, interferon alfa-2b (Intron A) or interferon alfa-2a (Roferon-A).

Exemplary polyclonal or monoclonal antibodies include, but are not limited to, trastuzumab (Herceptin); ofatumumab (Arzerra); bevacizumab (Avastin); rituximab (Rituxan); cetuximab (Erbitux); panitumumab (Vectibix); tositumomab/odine131 tositumomab (Bexxar); alemtuzumab (Campath); ibritumomab (Zevalin; In-111; Y-90 Zevalin); gemtuzumab (Mylotarg); eculizumab (Soliris) ordenosumab.

Exemplary EGFR inhibitors include, but are not limited to, gefitinib (Iressa); lapatinib (Tykerb); cetuximab (Erbitux); erlotinib (Tarceva); panitumumab (Vectibix); PKI-166; canertinib (CI-1033); matuzumab (Emd7200) or EKB-569.

Exemplary HER2 inhibitors include, but are not limited to, trastuzumab (Herceptin); lapatinib (Tykerb) or AC-480.

Exemplary histone deacetylase inhibitors include, but are not limited to, vorinostat (Zolinza), valproic acid, romidepsin, entinostat abexinostat, givinostat, and mocetinostat.

Exemplary hormones include, but are not limited to, tamoxifen (Soltamox; Nolvadex); raloxifene (Evista); megestrol (Megace); leuprolide (Lupron; Lupron Depot; Eligard; Viadur); fulvestrant (Faslodex); letrozole (Femara); triptorelin (Trelstar LA; Trelstar Depot); exemestane (Aromasin); goserelin (Zoladex); bicalutamide (Casodex); anastrozole (Arimidex); fluoxymesterone (Androxy; Halotestin); medroxyprogesterone (Provera; Depo-Provera); estramustine (Emcyt); flutamide (Eulexin); toremifene (Fareston); degarelix (Firmagon); nilutamide (Nilandron); abarelix (Plenaxis); or testolactone (Teslac).

Exemplary mitotic inhibitors include, but are not limited to, paclitaxel (Taxol; Onxol; Abraxane); docetaxel (Taxotere); vincristine (Oncovin; Vincasar PFS); vinblastine (Velban); etoposide (Toposar; Etopophos; VePesid); teniposide (Vumon); ixabepilone (Ixempra); nocodazole; epothilone; vinorelbine (Navelbine); camptothecin (CPT); irinotecan (Camptosar); topotecan (Hycamtin); amsacrine or lamellarin D (LAM-D).

Exemplary phosphatidyl-inositol-3 kinase (PI3K) inhibitors include wortmannin an irreversible inhibitor of PI3K, demethoxyviridin a derivative of wortmannin, LY294002, a reversible inhibitor of PI3K; BKM120 (Buparlisib); Idelalisib (a PI3K Delta inhibitor); duvelisib (IPI-145, an inhibitor of PI3K delta and gamma); alpelisib (BYL719), an alpha-specific PI3K inhibitor; TGR 1202 (previously known as RP5264), an oral PI3K delta inhibitor; and copanlisib (BAY 80-6946), an inhibitor PI3Kα,δ isoforms predominantly.

Exemplary Akt inhibitors include, but are not limited to miltefosine, AZD5363, GDC-0068, MK2206, Perifosine, RX-0201, PBI-05204, GSK2141795, and SR13668.

Exemplary MTOR inhibitors include, but are not limited to, everolimus (Afinitor) or temsirolimus (Torisel); rapamune, ridaforolimus; deforolimus (AP23573), AZD8055 (AstraZeneca), OSI-027 (OSI), INK-128, BEZ235, PI-103, Torin1, PP242, PP30, Ku-0063794, WAY-600, WYE-687, WYE-354, and CC-223.

Exemplary proteasomal inhibitors include, but are not limited to, bortezomib (PS-341), ixazomib (MLN 2238), MLN 9708, delanzomib (CEP-18770), carfilzomib (PR-171), YU101, oprozomib (ONX-0912), marizomib (NPI-0052), and disufiram.

Exemplary PARP inhibitors include, but are not limited to, olaparib, iniparib, velaparib, BMN-673, BSI-201, AG014699, ABT-888, GP121016, MK4827, INO-1001, CEP-9722, PJ-34, Tiq-A, Phen, PF-01367338 and combinations thereof.

Exemplary Ras/MAPK pathway inhibitors include, but are not limited to, trametinib, selumetinib, cobimetinib, CI-1040, PD0325901, AS703026, R04987655, R05068760, AZD6244, GSK1120212, TAK-733, U0126, MEK162, and GDC-0973.

Exemplary centrosome declustering agents include, but are not limited to, griseofulvin; noscapine, noscapine derivatives, such as brominated noscapine (e.g., 9-bromonoscapine), reduced bromonoscapine (RBN), N-(3-brormobenzyl) noscapine, aminonoscapine and water-soluble derivatives thereof; CW069; the phenanthridene-derived poly(ADP-ribose) polymerase inhibitor, PJ-34; N2-(3-pyridylmethyl)-5-nitro-2-furamide, N2-(2-thienylmethyl)-5-nitro-2-furamide, and N2-benzyl-5-nitro-2-furamide.

Exemplary multi-kinase inhibitors include, but are not limited to, regorafenib; sorafenib (Nexavar); sunitinib (Sutent); BIBW 2992; E7080; Zd6474; PKC-412; motesanib; or AP24534.

Exemplary serine/threonine kinase inhibitors include, but are not limited to, ruboxistaurin; eril/easudil hydrochloride; flavopiridol; seliciclib (CYC202; Roscovitrine); SNS-032 (BMS-387032); Pkc412; bryostatin; KAI-9803; SF1126; VX-680; Azd1152; Arry-142886 (AZD-6244); SCIO-469; GW681323; CC-401; CEP-1347 or PD 332991.

Exemplary tyrosine kinase inhibitors include, but are not limited to, erlotinib (Tarceva); gefitinib (Iressa); imatinib (Gleevec); sorafenib (Nexavar); sunitinib (Sutent); trastuzumab (Herceptin); bevacizumab (Avastin); rituximab (Rituxan); lapatinib (Tykerb); cetuximab (Erbitux); panitumumab (Vectibix); everolimus (Afinitor); alemtuzumab (Campath); gemtuzumab (Mylotarg); temsirolimus (Torisel); pazopanib (Votrient); dasatinib (Sprycel); nilotinib (Tasigna); vatalanib (Ptk787; ZK222584); CEP-701; SU5614; MLN518; XL999; VX-322; Azd0530; BMS-354825; SKI-606 CP-690; AG-490; WHI-P154; WHI-P131; AC-220; or AMG888.

Exemplary VEGF/VEGFR inhibitors include, but are not limited to, bevacizumab (Avastin); sorafenib (Nexavar); sunitinib (Sutent); ranibizumab; pegaptanib; or vandetinib.

Exemplary microtubule targeting drugs include, but are not limited to, paclitaxel, docetaxel, vincristin, vinblastin, nocodazole, epothilones and navelbine.

Exemplary topoisomerase poison drugs include, but are not limited to, teniposide, etoposide, adriamycin, camptothecin, daunorubicin, dactinomycin, mitoxantrone, amsacrine, epirubicin and idarubicin.

Exemplary taxanes or taxane derivatives include, but are not limited to, paclitaxel and docetaxol.

Exemplary general chemotherapeutic, anti-neoplastic, anti-proliferative agents include, but are not limited to, altretamine (Hexalen); isotretinoin (Accutane; Amnesteem; Claravis; Sotret); tretinoin (Vesanoid); azacitidine (Vidaza); bortezomib (Velcade) asparaginase (Elspar); levamisole (Ergamisol); mitotane (Lysodren); procarbazine (Matulane); pegaspargase (Oncaspar); denileukin diftitox (Ontak); porfimer (Photofrin); aldesleukin (Proleukin); lenalidomide (Revlimid); bexarotene (Targretin); thalidomide (Thalomid); temsirolimus (Torisel); arsenic trioxide (Trisenox); verteporfin (Visudyne); mimosine (Leucenol); (1M tegafur-0.4 M 5-chloro-2,4-dihydroxypyrimidine-1 M potassium oxonate) or lovastatin.

In certain embodiments, PD-L1 inhibition is carried out in combination with CD3 stimulation (e.g., by co-incubation with a cell expressing membrane CD3) before, at the same time, or after treatment with an anti-PD-L1 antibody. For example, in one embodiment, a method of enhancing an antigen-specific T cell response includes the step of contacting a T cell with an anti-PD-L1 antibody described herein, and a CD3-expressing cell, such that an antigen-specific T cell response is enhanced, and the PD-L1-mediated immunosuppression is reduced. Any suitable indicator of an antigen-specific T cell response can be used to measure the antigen-specific T cell response. Non-limiting examples of such suitable indicators include increased T cell proliferation in the presence of the antibody and/or increase cytokine production in the presence of the antibody. In a preferred embodiment, interleukin-2 and/or interferon-γ production by the antigen-specific T cell is enhanced.

In some embodiments, the anti-PD-L1 antibody described herein may also be used in combination with bispecific antibodies that target Fcα or Fcγ receptor-expressing effectors cells to tumor cells (see, e.g., U.S. Pat. Nos. 5,922,845 and 5,837,243). Such bispecific antibodies can be used to target two separate antigens. For example, anti-Fc receptor/anti-tumor antigen (e.g., Her-2/neu) bispecific antibodies have been used to target macrophages to sites of tumor. This targeting may more effectively activate tumor specific responses. The T cell arm of these responses would be augmented by the inhibition of PD-L1. Alternatively, antigen may be delivered directly to DCs by the use of bispecific antibodies that bind to tumor antigen and a dendritic cell specific cell surface marker.

In all of the above methods, PD-L1 inhibition can be combined with other forms of immunotherapy such as cytokine treatment (e.g., interferons, GM-CSF, G-CSF, IL-2), or bispecific antibody therapy using two different binding specificities to provide enhanced presentation of tumor antigens.

In some embodiments, the additional therapeutic agent for use in any of the foregoing methods of treatment, uses of an antigen binding molecule or uses of a pharmaceutical composition is an immunostimulatory agent selected from (a) an agent that blocks signaling of an inhibitory receptor (immune checkpoint) of an immune cell or a ligand thereof (immune checkpoint inhibitor) or a nucleic acid encoding such agent; (b) an agonist to a stimulatory receptor of an immune cell or a nucleic acid encoding such agonist; (c) a cytokine or a nucleic acid encoding a cytokine; (d) an oncolytic virus or a nucleic acid encoding an oncolytic virus; (e) a T cell expressing a chimeric antigen receptor; (f) a bi- or multi-specific T cell directed antibody or a nucleic acid encoding such antibody; (g) an anti-TGF-β antibody or a nucleic acid encoding such antibody; (h) a TGF-β trap or a nucleic acid encoding such trap; (i) a vaccine to a cancer-associated antigen, including such antigen or a nucleic acid encoding such antigen, (j) a cell therapy, and (k) combinations thereof. In some embodiments, the additional therapeutic agent is an agent that blocks signaling of an inhibitory receptor of an immune cell or a ligand thereof or a nucleic acid encoding such agent, and the inhibitory receptor or ligand thereof is selected from TIGIT, CTLA-4, PD-1, PD-L1, PD-L2, LAG-3, TIM-3, neuritin, BTLA, CECAM-1, CECAM-5, IL-1R8, VISTA, LAIR1, LILRB1, LILRB2, LILRB3, LILRB4, LILRB5, CD96, CD112R, CD 160, 2B4, TGFβ-R, KIR, NKG2A, MICA, MICB, A2aR, A2bR, and combinations thereof. In some embodiments, the additional therapeutic agent is an agonist to a stimulatory receptor of an immune cell or a nucleic acid encoding such agonist, and the stimulatory receptor of an immune cell is selected from OX40, CD2, CD16, CD27, CDS, ICAM-1, LFA-1 (CD11a/CD18), ICOS (CD278), 4-1BB (CD 137), GITR, CD28, CD30, CD40, BAFFR, HVEM, CD7, LIGHT, KG2C, SLAMF7, NKG2C, NKG2D, NKp46, NKp80, CD 160, B7-H3, CD83 ligand, and combinations thereof. In some embodiments, the additional therapeutic agent is a cytokine or a nucleic acid encoding a cytokine selected from IL-2, IL-5, IL-7, IL-12, IL-15, IL-2I, and combinations thereof. In some embodiments, the additional therapeutic agent is an oncolytic virus or a nucleic acid encoding an oncolytic virus selected from herpes simplex virus, vesicular stomatitis virus, adenovirus, Newcastle disease virus, vaccinia virus, a maraba virus, and combinations thereof. In some embodiments, the additional therapeutic agent is a cell therapy. A cell therapy may include a T cell, NK cell, or macrophage with a chimeric antigen receptor (CAR). In some embodiments, the cell therapy includes a bi- or multi-specific T cell directed antibody.

In certain embodiments, the present invention provides a method of treating a disease or disorder, e.g., cancer, in a subject. The method includes administering antigen binding molecules, e.g., anti-PD-L1 antibodies or antigen binding fragment thereof, of the present invention alone or in combination with a second one or more additional therapeutical agents into the subject, wherein the subject has previously received a treatment with a first one or more additional therapeutical agents. In certain embodiment, the treatment with the first one or more additional therapeutical agents may show low efficacy in treating the disease in the subject. For example, the treatment with the first one or more therapeutic agents may be treatment with an anti-PD1 antibody, to which the subject may show resistance. In some embodiments, the second one or more additional therapeutic agents are the same as the first one or more additional therapeutic agents. In some embodiments, the second one or more additional therapeutic agents are different to the first one or more additional therapeutic agents.

In certain embodiment, the immune checkpoint inhibitor is an antibody that interacts specifically with an immune checkpoint. In some embodiments, the additional therapeutic agent comprises an immunostimulatory agent. In some aspects, the immune checkpoint inhibitor is an and -CTLA-4 antibody (e.g., ipilimumab), and combinations thereof. In some aspects, the immune checkpoint inhibitor is pembrolizumab. In some aspects, the immune checkpoint inhibitor is nivolumab. In some aspects, the immune checkpoint inhibitor is atezolizumab.

In some embodiments, the anti-PD-L1 antibody is administered in combination with or concurrently with an immunogenic agent. Non-limiting examples of immunogenic agents include cancer cells, tumor vaccines, and purified tumor antigens (including recombinant proteins, peptides, and carbohydrate molecules); an oncolytic virus; cells transfected with genes encoding immune stimulating cytokines etc.

In certain embodiments, the anti-PD-L1 antibody is administered together with an antigen of interest or an antigen known to be present in the subject to be treated (e.g., a tumor-bearing or virus-bearing subject) to enhance antigen-specific immunity. When an anti-PD-L1 antibody is administered together with another agent, the two can be administered separately or simultaneously.

In certain embodiments, the anti-PD-L1 antibodies described herein may be used to enhance antigen-specific immune responses by co-administration of one or more of any of these antibodies with an antigen of interest (e.g., a vaccine). Accordingly, in one embodiment, a method for enhancing an immune response to an antigen in a subject, includes the steps of administering to the subject: (i) the antigen; and (ii) an PD-L1-based antibody such that an immune response to the antigen in the subject is enhanced. The antigen can be, for example, a tumor antigen, a viral antigen, a bacterial antigen or an antigen from a pathogen. Non-limiting examples of such antigens include those discussed in the sections above, such as the tumor antigens (or tumor vaccines) discussed above, or antigens from the viruses, bacteria or other pathogens described above.

In view of the benefits associated with synergistic active agent compositions, in certain embodiments, each of the anti-PD-L1 antibody and the other active agents are administered to a subject in need thereof at subtherapeutic doses relative to the doses used in monotherapies with the same.

In certain embodiments, PD-L1 inhibition is combined with standard cancer treatments (e.g., surgery, radiation, and chemotherapy). In these instances, it may be possible to reduce the dose of chemotherapeutic reagent administered. It is believed that the combined use of PD-L1 inhibition and chemotherapy can enhance apoptosis and increase tumor antigen presentation for cytotoxic immunity. Other synergistic combination therapies include PD-L1 inhibition in combination with radiation, surgery or hormone deprivation or inhibition. Each of these protocols creates a source of tumor antigen in the host.

The additional therapeutical agent may be administered prior to, concurrent with, or after the administration of an antigen binding molecule of the present invention; (for purposes of the present disclosure, such administration regimens are considered the administration of an antigen binding molecule “in combination with” an additional therapeutically active component).

The present invention includes pharmaceutical compositions in which an antigen binding molecule of the present invention is co-formulated with one or more of the additional therapeutical agents as described elsewhere herein.

### V. Nucleic Acids and Host Cells for Expressing Anti-PD-L1 Antibodies

In another aspect, the present invention provides nucleic acids encoding the antigen binding molecules, e.g., anti-PD-L1 antibodies or antigen binding fragments thereof, of the present invention, and expression vectors comprising such nucleic acids. In some embodiments, nucleic acids encode an HCVR and/or LCVR fragment of an antibody or fragment in accordance with the embodiments described herein, or any of the other antibodies and antibody fragments described herein.

DNA encoding an antigen binding site in a monoclonal antibody can be isolated and sequenced from the hybridoma cells using conventional procedures (e.g., by using oligonucleotide probes that are capable of binding specifically to genes encoding the heavy and light chains of the monoclonal antibodies). Alternatively, amino acid sequences from immunoglobulins of interest may be determined by direct protein sequencing, and suitable encoding nucleotide sequences can be designed according to a universal codon table. In other cases, nucleotide and amino acid sequences of antigen binding sites or other immunoglobulin sequences, including constant regions, hinge regions and the like may be obtained from published sources well known in the art.

Expression vectors may be used to synthesize the antibodies of the present disclosure in cultured cells in vitro or they may be directly administered to a patient to express the antibodies of the present disclosure in vivo or ex vivo. As used herein, an “expression vector” refers to a viral or non-viral vector comprising a polynucleotide encoding one or more antibodies of the present disclosure in a form suitable for expression from the polynucleotide(s) in a host cell for antibody preparation purposes or for direct administration as a therapeutic agent.

A nucleic acid sequence is “operably linked” to another nucleic acid sequence when the former is placed into a functional relationship with the latter. For example, a DNA for a presequence or signal peptide is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a signal peptide, contiguous and in reading phase. However, enhancers do not have to be contiguous. Linking is accomplished by ligation at convenient restriction sites. If such sites do not exist, synthetic oligonucleotide adaptors or linkers may be used in accordance with conventional practice.

Nucleic acid sequences for expressing the antibodies of the present disclosure typically include an N terminal signal peptide sequence, which is removed from the mature protein. Since the signal peptide sequences can affect the levels of expression, the polynucleotides may encode any one of a variety of different N-terminal signal peptide sequences. It will be appreciated by those skilled in the art that the design of the expression vector can depend on such factors as the choice of the host cell to be transformed, the level of expression of protein desired, and the like.

The above described “regulatory sequences” refer to DNA sequences necessary for the expression of an operably linked coding sequence in one or more host organisms. The term “regulatory sequences” is intended to include promoters, enhancers and other expression control elements (e.g., polyadenylation signals). Regulatory sequences include those which direct constitutive expression of a nucleotide sequence in many types of host cells or those which direct expression of the nucleotide sequence only in certain host cells (e.g., tissue-specific regulatory sequences). Expression vectors generally contain sequences for transcriptional termination, and may additionally contain one or more elements positively affecting mRNA stability.

The expression vector contains one or more transcriptional regulatory elements, including promoters and/or enhancers, for directing the expression of antibodies of the present disclosure. A promoter comprises a DNA sequence that functions to initiate transcription from a relatively fixed location in regard to the transcription start site. A promoter contains core elements required for basic interaction of RNA polymerase and transcription factors, and may operate in conjunction with other upstream elements and response elements.

As used herein, the term “promoter” is to be construed broadly so as to include e.g., transcriptional regulatory elements (TREs) from genomic genes or chimeric TREs therefrom, including the TATA box or initiator element for accurate transcription initiation, with or without additional TREs (i.e., upstream activating sequences, transcription factor binding sites, enhancers, and silencers) which regulate activation or repression of genes operably linked thereto in response to developmental and/or external stimuli, and trans-acting regulatory proteins or nucleic acids. A promoter may contain a genomic fragment or it may contain a chimera of one or more TREs combined together.

Preferred promoters are those capable of directing high-level expression in a target cell of interest. The promoters may include constitutive promoters (e.g., HCMV, SV40, elongation factor-la (EF-1α)) or those exhibiting preferential expression in a particular cell type of interest. Enhancers generally refer to DNA sequences that function away from the transcription start site and can be either 5′ or 3′ to the transcription unit. Furthermore, enhancers can be within an intron as well as within the coding sequence. They are usually between 10 and 300 bp in length, and they function in cis. Enhancers function to increase and/or regulate transcription from nearby promoters. Preferred enhancers are those directing high-level expression in the antibody producing cell. Cell or tissue-specific transcriptional regulatory elements (TREs) can be incorporated into expression vectors to restrict expression to desired cell types. An expression vector may be designed to facilitate expression of the antibodies of the present disclosure in one or more cell types.

To co-express the individual chains of the antibodies of the present disclosure, a suitable splice donor and splice acceptor sequences may be incorporated for expressing both products. Alternatively, an internal ribosome binding sequence (IRES) or a 2A peptide sequence, may be employed for expressing multiple products from one promoter. An IRES provides a structure to which the ribosome can bind that does not need to be at the 5′ end of the mRNA. It can therefore direct a ribosome to initiate translation at a second initiation codon within a mRNA, allowing more than one polypeptide to be produced from a single mRNA. A 2A peptide contains short sequences mediating co-translational self-cleavage of the peptides upstream and downstream from the 2A site, allowing production of two different proteins from a single transcript in equimolar amounts. CHYSEL is a non-limiting example of a 2A peptide, which causes a translating eukaryotic ribosome to release the growing polypeptide chain that it is synthesizing without dissociating from the mRNA. The ribosome continues translating, thereby producing a second polypeptide.

An expression vector may comprise a viral vector or a non-viral vector. A viral vector may be derived from an adeno-associated virus (AAV), adenovirus, herpesvirus, vaccinia virus, poliovirus, poxvirus, a retrovirus (including a lentivirus, such as HIV-1 and HIV-2), Sindbis and other RNA viruses, alphavirus, astrovirus, coronavirus, orthomyxovirus, papovavirus, paramyxovirus, parvovirus, picornavirus, togaviruses and the like. A non-viral vector is simply a “naked” expression vector that is not packaged with virally derived components (e.g., capsids and/or envelopes).

In certain cases, these vectors may be engineered to target certain diseases or cell populations by using the targeting characteristics inherent to the virus vector or engineered into the virus vector. Specific cells may be “targeted” for delivery of polynucleotides, as well as expression. Thus, the term “targeting”, in this case, may be based on the use of endogenous or heterologous binding agents in the form of capsids, envelope proteins, antibodies for delivery to specific cells, the use of tissue-specific regulatory elements for restricting expression to specific subset(s) of cells, or both.

In some embodiments, expression of the antibody chains is under the control of the regulatory element such as a tissue specific or ubiquitous promoter. In some embodiments, a ubiquitous promoter such as a CMV promoter, CMV-chicken beta-actin hybrid (CAG) promoter, a tissue specific or tumor-specific promoter to control the expression of a particular antibody heavy or light chain or single-chain derivative therefrom.

Non-viral expression vectors can be utilized for non-viral gene transfer, either by direct injection of naked DNA or by encapsulating the antibody-encoding polynucleotides in liposomes, microparticles, microcapsules, virus-like particles, or erythrocyte ghosts. Such compositions can be further linked by chemical conjugation to targeting domains to facilitate targeted delivery and/or entry of nucleic acids into desired cells of interest. In addition, plasmid vectors may be incubated with synthetic gene transfer molecules such as polymeric DNA-binding cations like polylysine, protamine, and albumin, and linked to cell targeting ligands such as asialoorosomucoid, insulin, galactose, lactose or transferrin.

Alternatively, naked DNA may be employed. Uptake efficiency of naked DNA may be improved by compaction or by using biodegradable latex beads. Such delivery may be improved further by treating the beads to increase hydrophobicity and thereby facilitate disruption of the endosome and release of the DNA into the cytoplasm.

### VI. Methods for Producing Anti-PD-L1 Antibodies

In another aspect, the present invention provides host cells transformed with the anti-PD-L1 HCVRs and/or LCVRs encoding nucleic acids or expression vectors. The host cells can be any bacterial or eukaryotic cell capable of expressing the anti-PD-L1 HCVRs and/or LCVRs encoding nucleic acids or expression vectors or any of the other co-administered antibodies or antagonists described herein.

In another aspect, a method of producing an antibody of the present disclosure comprises culturing a host cell transformed with one or anti-PD-L1 HCVRs and/or LCVRs encoding nucleic acids or expression vectors under conditions that allows production of the antibody or fragment, and purifying the antibody from the cell.

In a further aspect, the present invention provides a method for producing an antibody comprising culturing a cell transiently or stably expressing one or more constructs encoding one or more polypeptide chains in the antibody; and purifying the antibody from the cultured cells. Any cell capable of producing a functional antibody may be used. In preferred embodiments, the antibody-expressing cell is of eukaryotic or mammalian origin, preferably a human cell. Cells from various tissue cell types may be used to express the antibodies. In other embodiments, the cell is a yeast cell, an insect cell or a bacterial cell. Preferably, the antibody-producing cell is stably transformed with a vector expressing the antibody.

One or more expression vectors encoding the antibody heavy or light chains can be introduced into a cell by any conventional method, such as by naked DNA technique, cationic lipid-mediated transfection, polymer-mediated transfection, peptide-mediated transfection, virus-mediated infection, physical or chemical agents or treatments, electroporation, etc. In addition, cells may be transfected with one or more expression vectors for expressing the antibody along with a selectable marker facilitating selection of stably transformed clones expressing the antibody. The antibodies produced by such cells may be collected and/or purified according to techniques known in the art, such as by centrifugation, chromatography, etc.

Examples of suitable selectable markers for mammalian cells include dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are CHO DHFR cells and mouse LTV cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, mycophenolic acid, or hygromycin. The three examples employ bacterial genes under eukaryotic control to convey resistance to the appropriate drug G418 or neomycin (geneticin), xgpt (mycophenolic acid) or hygromycin, respectively. Others include the neomycin analog G418 and puromycin.

Exemplary antibody-expressing cells include human Jurkat, human embryonic kidney (HEK) 293, Chinese hamster ovary (CHO) cells, mouse WEHI fibrosarcoma cells, as well as unicellular protozoan species, such as Leishmania tarentolae. In addition, stably transformed, antibody producing cell lines may be produced using primary cells immortalized with c-myc or other immortalizing agents.

In one embodiment, the cell line comprises a stably transformed Leishmania cell line, such as Leishmania tarentolae. Leishmania are known to provide a robust, fast-growing unicellular host for high level expression of eukaryotic proteins exhibiting mammalian-type glycosylation patterns. A commercially available Leishmania eukaryotic expression kit is available (Jena Bioscience GmbH, Jena, Germany).

In some embodiments, the cell lines express at least 1 mg, at least 2 mg, at least 5 mg, at least 10 mg, at least 20 mg, at least 50 mg, or at least 100 mg of the antibody/liter of culture.

The antibodies in the present invention may be isolated from antibody expressing cells following culture and maintenance in any appropriate culture medium, such as RPMI, DMEM, and AIM V®. The antibodies can be purified using conventional protein purification methodologies (e.g., affinity purification, chromatography, etc.), including the use of Protein-A or Protein-G immunoaffinity purification. In some embodiments, antibodies are engineered for secretion into culture supernatants for isolation therefrom.

### VII. Pharmaceutical Compositions and Dosing Methodologies

In one aspect, a pharmaceutical composition of the present invention includes an antigen binding molecule, e.g., an PD-L1 antibody or antigen binding fragment(s) thereof as described herein in combination with a pharmaceutically acceptable carrier. In other embodiments, the PD-L1 antibody or antigen binding fragment(s) thereof are administered in combination with a pharmaceutically acceptable carrier. Anti-PD-L1 compositions may include one or more different antibodies, one or more multispecific antibodies, one or more fusion proteins, one or more immunoconjugates, or a combination thereof as described herein.

The present invention provides pharmaceutical compositions comprising the antigen binding molecules of the present invention. The pharmaceutical compositions of the invention are formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antigen binding molecule administered to a patient may vary depending upon the age and the size of the patient, target disease, conditions, route of administration, and the like. The preferred dose is typically calculated according to body weight or body surface area. When a bispecific antigen binding molecule of the present invention is used for therapeutic purposes in an adult patient, it may be advantageous to intravenously administer the bispecific antigen binding molecule of the present invention normally at a single dose of about 0.01 to about 20 mg/kg body weight, more preferably about 0.02 to about 7, about 0.03 to about 5, or about 0.05 to about 3 mg/kg body weight. Depending on the severity of the condition, the frequency and the duration of the treatment can be adjusted. Effective dosages and schedules for administering a bispecific antigen binding molecule may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

In another aspect, a method for treating a cell proliferative disorder, such as cancer, a chronic infection, or an immunologically compromised disease state includes administering to a subject in need thereof a pharmaceutical composition containing an anti-PD-L1 antibody or antigen binding fragment as described herein in combination with a pharmaceutically acceptable carrier. In some embodiments, the method restores, potentiates or enhances the activity of lymphocytes in a subject in need thereof. In certain preferred embodiments, the antibody or fragment is a human or humanized anti-PD-L1 antibody that reduces or abrogates signaling through the PD-L1.

In some embodiments, administration of the pharmaceutical composition increases the activity of lymphocytes (e.g., T cells) in patients having a disease in which increased lymphocyte activity is beneficial or which is caused or characterized by immunosuppression, immunosuppressive cells, or, e.g., PD-L1 over expression generated by CD4 T cells, CD8 T cells). The methods described herein are particularly useful, e.g., in patients having a solid tumor in which it is suspected the tumor microenvironment may contribute to the lack of recognition by the immune system (immune escape). The tumor may, for example, be characterized by PD-L1-expressing (or overexpressing) immune cells, e.g., CD4 T cells, CD8 T cells, T-regs, B cells.

In certain embodiments, the methods and compositions are utilized for the treatment of a variety of cancers and other proliferative diseases. Because these methods serve to reduce PD-L1 levels, which can inhibit the anti-tumor activity of lymphocytes, they are applicable to a very broad range of cancers, particularly solid tumors where PD-L1 in the tumor microenvironment is known to suppress anti-tumor immune responses.

Non-limiting cancers for treatment using the antigen binding molecules, e.g., anti-PD-L1 antibodies or antigen binding fragments thereof, of the present invention include, for example, liver cancer, bone cancer, pancreatic cancer, skin cancer, cancer of the head or neck, breast cancer, lung cancer, non-small cell lung cancer (NSCLC), castrate resistant prostate cancer (CRPC), melanoma, uterine cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers including those induced by asbestos, hematologic malignancies including, for example, multiple myeloma, B-cell lymphoma, Hodgkin lymphoma/primary mediastinal B-cell lymphoma, non-Hodgkin's lymphomas, acute myeloid lymphoma, chronic myelogenous leukemia, chronic lymphoid leukemia, follicular lymphoma, diffuse large B-cell lymphoma, Burkitt's lymphoma, immunoblastic large cell lymphoma, precursor B-lymphoblastic lymphoma, mantle cell lymphoma, acute lymphoblastic leukemia, mycosis fungoides, anaplastic large cell lymphoma, T-cell lymphoma, and precursor T-lymphoblastic lymphoma, or any combination of these cancers. The present disclosure is also applicable to treatment of metastatic cancers. Patients can be tested or selected for one or more of the above-described clinical attributes prior to, during or after treatment.

In one embodiment, the anti-PD-L1 antibody is administered an amount effective to achieve and/or maintain in an individual (e.g., for 1, 2, 3, 4 weeks, and/or until the subsequent administration of antigen binding compound) a blood concentration of at least the EC₅₀, optionally the EC₇₀, optionally substantially the EC₁₀₀, for neutralization of the enzymatic activity of PD-L1. In one embodiment, the active amount of anti-PD-L1 antibody is an amount effective to achieve the EC₅₀, optionally the EC₇₀, optionally substantially the EC₁₀₀, for neutralization of the enzymatic activity of PD-L1 in an extravascular tissue of an individual. In one embodiment, the active amount of anti-PD-L1 antibody is an amount effective to achieve (or maintain) in an individual the EC₅₀, optionally the EC₇₀, optionally substantially the EC₁₀₀, for inhibition of neutralize the enzymatic activity of PD-L1.

Optionally, in one embodiment, in contrast to some antibodies that are directed to the depletion of PD-L1-expressing tumor cells by ADCC (which, e.g., can provide full efficacy at concentrations equal or substantially lower than that which provides receptor saturation), the anti-PD-L1 antibody is a mainly blocker (no substantial Fcγ receptor-mediated activity) and is administered in an amount effective to neutralize the enzymatic activity of PD-L1 for a desired period of time, e.g., 1 week, 2 weeks, a month, until the next successive administration of anti-PD-L1 antibody.

In one embodiment, the anti-PD-L1 antibody is administered in an amount effective to achieve and/or maintain (e.g., for 1, 2, 3, 4 weeks, and/or until the subsequent administration of anti-PD-L1 antibody) in an individual a blood concentration of at least the EC₅₀, optionally the EC₇₀, optionally substantially the EC₁₀₀, for inhibition of PD-L1-mediated immunosuppression on T cells. In one embodiment, the amount of anti-PD-L1 antibody is an amount effective to achieve (or maintain) the EC₅₀, optionally the EC₇₀, optionally substantially the EC₁₀₀, for inhibition of PD-L1-mediated immunosuppression in an extravascular tissue of an individual.

In one embodiment, provided is a method for treating or preventing cancer in an individual, the method comprising administering to an individual having disease an anti-PD-L1 antibody in an amount that achieves or maintains for a specified period of time a concentration in circulation, optionally in an extravascular tissue of interest (e.g., the tumor or tumor environment), that is higher than the concentration required for 50%, 70%, or full (e.g., 90%) receptor saturation PD-L1-expressing cells in circulation (for example as assessed in PBMC). Optionally the concentration achieved is at least 20%, 50% or 100% higher than the concentration required for the specified receptor saturation.

In one embodiment, provided is a method for treating or preventing cancer in an individual, the method comprising administering to the individual an anti-PD-L1 antibody in an amount that achieves or maintains for a specified period of time a concentration in circulation, optionally in an extravascular tissue of interest (e.g., the tumor or tumor environment), that is higher than the EC₅₀, optionally EC₇₀ or optionally EC₁₀₀, for binding to PD-L1-expressing cells. Optionally the concentration achieved is at least 20%, 50% or 100% higher than the EC₅₀, optionally EC₇₀ or optionally EC₁₀₀, for binding to PD-L1-expressing cells.

In any embodiment, the antibody can for example have an EC₅₀, optionally EC₇₀ or optionally EC₁₀₀, for binding to PD-L1-expressing cells in human PBMC of between 0.5-100 ng/ml, optionally 1-100 ng/ml, optionally 30-100 ng/ml, e.g., about 30-90 ng/ml. For example, the EC₅₀ may be about 30, 37, 39, 43, 57, 58, 61, 62, 90, 95, 143 ng/ml.

The EC₅₀ for neutralization of the enzymatic activity of PD-L1 with the anti-PD-L1 antibody can be for example between about 0.01 μg/ml and 1 μg/ml, optionally between 0.1 μg/ml and 10 μg/ml, optionally between 0.1 μg/ml and 1 μg/ml. For example, the EC₅₀ may be about 0.1 μg/ml, about 0.2 μg/ml or about 0.3 μg/ml. Thus, an amount of this anti-PD-L1 antibody is for example administered so at to achieve and/or maintain a blood concentration of at least 0.1 μg/ml, optionally at least 0.2 μg/ml, optionally at least 1 μg/ml, or optionally at least 2 μg/ml.

When tissues outside of the vasculature are targeted (the tumor environment, e.g., in the treatment of solid tumors), an approximately 10-fold higher dose is typically believed to be needed, compared to the dose that provides the corresponding concentration in circulation. An amount of anti-PD-L1 antibody administered so at to achieve (and/or maintain) a concentration in circulation (blood) of about 1 μg/ml, 2 μg/ml, 10 μg/ml, or 20 μg/ml is expected to achieve (and/or maintain) an extravascular tissue (e.g., tumor tissue) concentration of about 0.1 μg/ml, 0.2 μg/ml, 1 μg/ml, 2 μg/ml, respectively.

In one embodiment, an anti-PD-L1 antibody is for example administered in an amount so at to achieve and/or maintain a tissue (e.g., tumor environment) concentration of at least 0.1 μg/ml, optionally at least 0.2 μg/ml, optionally at least 1 μg/ml, or optionally at least 2 μg/ml. The antibody can for example be administered in an amount to achieve and/or maintained a blood concentration of at least about 1 μg/ml, 2 μg/ml, 10 μg/ml, or 20 μg/ml, e.g., between 1-100 μg/ml, 10-100 μg/ml, 1-50 μg/ml, 1-20 μg/ml, or 1-10 μg/ml. The amount administered can be adjusted to as to provide for maintenance of the desired concentration for the duration of a specified period of time following administration (e.g., 1, 2, 3, 4 weeks, etc.).

In some embodiments, an amount of anti-PD-L1 antibody is administered so as to obtain a concentration in blood (serum) or an extravascular tissue (e.g., tumor environment) that corresponds to at least the EC₇₀ or the EC₁₀₀ for neutralization of the enzymatic activity of PD-L1. The antibody can for example be administered in an amount to achieve and/or maintained a blood concentration or an extravascular tissue (e.g., tumor environment) of at least about 1 μg/ml, 2 μg/ml, 10 μg/ml, or 20 μg/ml.

EC₅₀, EC₇₀ and EC₁₀₀ values for a given PD-L1 antibody can be assessed for example in a cellular assay for blocking PD-1/PD-L1 interaction. “EC₅₀” with respect to neutralization of the enzymatic activity of PD-L1, refers to the concentration of anti-PD-L1 antibody which produces 50% of its maximum response or effect with respect to neutralization of the enzymatic activity.). “EC₇₀” with respect to neutralization of the enzymatic activity of PD-L1, refers to the concentration of anti-PD-L1 antibody which produces 70% of its maximum response or effect. “EC₁₀₀” with respect to neutralization of the enzymatic activity of PD-L1, refers to the efficient concentration of anti-PD-L1 antibody which produces its maximum response or effect with respect to such neutralization of the enzymatic activity. In certain embodiments and depending on the context, EC₅₀, EC₇₀, or EC₁₀₀, may be referred to as IC₅₀, IC₇₀, or IC₁₀₀, respectively, to reflect that the antigen binding molecule, e.g., the anti-PD-L1 antibody or the antigen binding fragment thereof inhibits the activities of the PD-L1. ICₓₓ refers to the concentration of a drug that is needed to inhibit a biological process by xx %.

In some embodiments, particularly for the treatment of solid tumors, the concentration achieved is designed to lead to a concentration in tissues (outside of the vasculature, e.g., in the tumor or tumor environment) that corresponds to at least the EC₅₀ for neutralization of the enzymatic activity, optionally at about, or at least about, the EC₁₀₀.

In one embodiment, the amount of anti-PD-L1 antibody is between 1 and 20 mg/kg body weight. In one embodiment, the amount is administered to an individual weekly, every two weeks, monthly or every two months.

In one embodiment, a method of treating a cancer in a subject in need thereof, includes administering to the individual an effective amount of an anti-PD-L1 antibody of the disclosure for at least one administration cycle (optionally at least 2, 3, 4 or more administration cycles), wherein the cycle is a period of eight weeks or less, wherein for each of the at least one cycles, one, two, three or four doses of the anti-PD-L1 antibody are administered at a dose of 1-20 mg/kg body weight. In one embodiment, the anti-PD-L1 antibody is administered by intravenous infusion.

Suitable treatment protocols for treating e.g., a human subject include, for example, administering to the patient an amount as disclosed herein of an anti-PD-L1 antibody, wherein the method includes at least one administration cycle in which at least one dose of the anti-PD-L1 antibody is administered. Optionally, at least 2, 3, 4, 5, 6, 7 or 8 doses of the anti-PD-L1 antibody are administered. In one embodiment, the administration cycle is between 2 weeks and 8 weeks.

In one embodiment, a method for treating or preventing a disease (e.g., a cancer, a solid tumor, a hematological tumor) in an individual, includes administering to the individual an anti-PD-L1 antibody that neutralizes the enzymatic activity of PD-L1 for at least one administration cycle, the administration cycle comprising at least a first and second (and optionally a 3rd, 4th, 5th 6th, 7th and/or 8th or further) administration of the anti-PD-L1 antibody, wherein the anti-PD-L1 antibody is administered in an amount effective to achieve, or to maintain between two successive administrations, a blood (serum) concentration of anti-PD-L1 antibody of at least 0.1 μg/ml, at least 0.2 μg/ml, at least 1 μg/ml, at least 2 μg/ml, at least 10 μg/ml, at least 20 μg/ml, between 1-100 μg/ml, between 1-50 μg/ml, between 1-20 μg/ml, between 1-10 μg/ml or a range between any of the aforementioned concentrations.

In one embodiment, a specified continuous blood concentration is maintained, wherein the blood concentration does not drop substantially below the specified blood concentration for the duration of the specified time period (e.g., between two administrations of antibody, number of weeks, 1 week, 2 weeks, 3 weeks, 4 weeks). In other words, although the blood concentration can vary during the specified time period, the specified blood concentration maintained represents a minimum or “trough” concentration.

In one embodiment, a therapeutically active amount of an anti-PD-L1 antibody is an amount of such antibody capable of providing (at least) the EC₅₀ concentration, optionally the EC₇₀ concentration optionally the EC₁₀₀ concentration, in blood and/or in a tissue for neutralization of the enzymatic activity of PD-L1 for a period of at least about 1 week, about 2 weeks, or about one month, following administration of the antibody.

Prior to or during a course of treatment with an anti-PD-L1 antibody of the disclosure, expression levels of PD-L1, and/or PD-1; percentages of PD-L1-expressing, and/or PD-1-expressing, can be assessed within and/or adjacent to a patient's tumor to assess whether the patient is suitable for treatment and is likely to respond to treatment. Increased levels or expression of the foregoing may indicate an individual is suitable for treatment with (e.g., likely to benefit from) an anti-PD-L1 antibody of the present disclosure.

In some embodiments, assessing the expression levels of PD-L1, and/or PD-1 within and/or adjacent to a patient's tumor the tissue sample includes the step of obtaining from a subject a biological sample of a human tissue selected from the group consisting of tissue from a cancer patient, e.g., cancer tissue, tissue proximal to or at the periphery of a cancer, cancer adjacent tissue, adjacent non-tumorous tissue or normal adjacent tissue, and expression levels of PD-L1, and/or PD-1 within the tissue. The expression levels or nucleotide concentrations from the patient can be comparing the level to a reference level, e.g., corresponding to a healthy individual.

In view of the foregoing, in certain embodiments, the method includes the steps of: (a) determining the expression levels of PD-L1, and/or PD-1 in the tumor environment, optionally within the tumor and/or within adjacent tissue, and upon a determination that tumor environment exhibits levels of PD-L1, and/or PD-1 that is/are increased compared to their corresponding reference level(s), (b) administering to the individual an anti-PD-L1 antibody.

In certain embodiments, determining the levels of PD-L1, and/or PD-L1 within the tumor environment includes the step of obtaining from the subject a biological containing cancer tissue and/or tissue proximal to or at the periphery of a cancer (e.g., cancer adjacent tissue, adjacent non-tumorous tissue or normal adjacent tissue), and detecting levels and/or relative percentages of PD-L1 and/or PD-1. PD-L1- and/or PD-1-expressing cells may include, for example, tumor cells, CD4 T cells, CD8 T cells, B cells, and combinations thereof. Expression levels of PD-L1, and/or PD-1 may be determined by evaluating their mRNA expression (by e.g., RT-PCR) or polypeptide expression (by e.g., western blotting, immunofluorescent staining) compared to a reference level corresponding to a healthy subject or compared to a reference level before treatment using techniques well known to those of ordinary skill in the art.

A subject with cancer can be treated with the anti-PD-L1 antibody with or without assessing the PD-L1, and PD-1 levels in the tumor microenvironment (e.g., on tumor cells, CD4 T cells, CD8 T cells, B cells).

A determination that a biological sample includes cells overexpressing PD-L1, and/or PD-1 compared to a reference, indicates that the subject has a cancer that may benefit from treatment with an agent that blocks PD-1/PD-L1 interaction. In some embodiments, the term “overexpressed” is used with reference to an PD-L1, and/or PD-1 polypeptide that is expressed in a substantial number of cells taken from a given patient, for example, on at least 10%, at least 20%, at least 30%, at least 40%, at least 50%, at least 60%, at least 70%, at least 80%, at least 90%, at least 95% or more of the tumor cells or lymphocytes taken from a subject.

In one embodiment, a method for the treatment or prevention of a cancer in an subject in need thereof includes the steps of: (a) detecting the percentage of cells and/or extent of expression corresponding to PD-L1, and/or PD-1 within the tumor environment, optionally within the tumor and/or within adjacent tissue, and upon a determination that the tumor environment includes cells overexpressing PD-L1, and/or PD-1, optionally at level(s) that are increased compared to suitable reference levels, (b) administering to the subject an anti-PD-L1 antibody. In one embodiment, the cells are tumor cells. In another embodiment, the cells within the tumor environment, tumor and/or adjacent tissue are non-malignant immune cells, e.g., T cells.

In some embodiments, determining the extent of PD-L1, and/or PD-1 expression within the tumor environment includes the step of obtaining from the individual a biological sample that comprises cancer tissue and/or tissue proximal to or at the periphery of a cancer (e.g., cancer adjacent tissue, adjacent non-tumorous tissue or normal adjacent tissue), contacting the cells with an antibody that binds an PD-L1 polypeptide, and/or PD-1 polypeptide and detecting the percentage of cells and/or the extent of expression corresponding to the PD-L1, and/or PD-1. In certain embodiments, expression of PD-L1, and/or PD-1 is evaluated by their cell surface expression using an immunohistochemistry assay.

The antibody compositions may be used as monotherapy or combined treatments with one or more other therapeutic agents, including agents normally utilized for the particular therapeutic purpose for which the antibody is being administered. See “Combination therapies” above. The additional therapeutic agent will normally be administered in amounts and treatment regimens typically used for that agent in a monotherapy for the particular disease or condition being treated. Such therapeutic agents include, but are not limited to anti-cancer agents and chemotherapeutic agents.

As described above, methods for using the pharmaceutical compositions described herein include the step of administering to a subject in need thereof an effective amount of the pharmaceutical composition according to the present disclosure.

Any suitable route or mode of administration can be employed for providing the patient with a therapeutically or prophylactically effective dose of the antibody. Exemplary routes or modes of administration include parenteral (e.g., intravenous, intraarterial, intramuscular, subcutaneous, intratumoral), oral, topical (nasal, transdermal, intradermal or intraocular), mucosal (e.g., nasal, sublingual, buccal, rectal, vaginal), inhalation, intralymphatic, intraspinal, intracranial, intraperitoneal, intratracheal, intravesical, intrathecal, enteral, intrapulmonary, intralymphatic, intracavital, intraorbital, intracapsular and transurethral, as well as local delivery by catheter or stent.

A pharmaceutical composition comprising an anti-PD-L1 antibody in accordance with the present disclosure may be formulated in any pharmaceutically acceptable carrier(s) or excipient(s). As used herein, the term “pharmaceutically acceptable carrier” includes any and all solvents, dispersion media, coatings, antibacterial and antifungal agents, isotonic and absorption delaying agents, and the like that are physiologically compatible. Pharmaceutical compositions may comprise suitable solid or gel phase carriers or excipients. Exemplary carriers or excipients include but are not limited to, calcium carbonate, calcium phosphate, various sugars, starches, cellulose derivatives, gelatin, and polymers such as polyethylene glycols. Exemplary pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the therapeutic agents.

In certain preferred embodiments, the therapeutically active agents can be incorporated into a pharmaceutical composition suitable for parenteral administration. Pharmaceutical composition for parenteral administration may be formulated by injection e.g., by bolus injection or continuous infusion.

Suitable buffers include but are not limited to, sodium succinate, sodium citrate, sodium phosphate or potassium phosphate. Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-Methionine (optimally 5-10 mM). Other suitable bulking agents include glycine, arginine, can be included as 0-0.05% polysorbate-80 (optimally 0.005-0.01%). Additional surfactants include but are not limited to polysorbate 20 and BRIJ surfactants.

Therapeutic agent preparations can be lyophilized and stored as sterile powders, preferably under vacuum, and then reconstituted in bacteriostatic water (containing, for example, benzyl alcohol preservative) or in sterile water prior to injection. The therapeutic agents in the pharmaceutical compositions may be formulated in a “therapeutically effective amount” or a “prophylactically effective amount”. A “therapeutically effective amount” refers to an amount effective, at dosages and for periods of time necessary, to achieve the desired therapeutic result. A therapeutically effective amount of an antibody or active agent may vary depending on the condition to be treated, the severity and course of the condition, the mode of administration, whether the antibody or agent is administered for preventive or therapeutic purposes, the bioavailability of the particular agent(s), the ability of the antibody to elicit a desired response in the individual, previous therapy, the age, weight and sex of the patient, the patient's clinical history and response to the antibody, the type of the antibody used, discretion of the attending physician, etc. A therapeutically effective amount is also one in which any toxic or detrimental effects of the recombinant vector is outweighed by the therapeutically beneficial effects. A “prophylactically effective amount” refers to an amount effective, at dosages and for periods of time necessary, to achieve the desired prophylactic result.

Preferably, the polypeptide domains utilized in the antibodies or other active agents described herein are derived from the same host in which they are to be administered in order to reduce inflammatory responses against the administered therapeutic agents. As suggested above, the therapeutic agent(s) are suitably administered to the subject at one time or over a series of treatments and may be administered to the patient at any time from diagnosis onwards. The PD-L1 antibody may be administered as the sole treatment or in conjunction with other active agents or therapies useful in treating the condition in question.

As a general proposition, a therapeutically effective amount or prophylactically effective amount of the PD-L1 antibody (or other active agent) will be administered in a range from about 1 ng/kg body weight/day to about 100 mg/kg body weight/day whether by one or more administrations. In a particular embodiment, each PD-L1 antibody or active agent is administered in the range of from about 1 ng/kg body weight/day to about 10 mg/kg body weight/day, about 1 ng/kg body weight/day to about 1 mg/kg body weight/day, about 1 ng/kg body weight/day to about 100 μg/kg body weight/day, about 1 ng/kg body weight/day to about 10 μg/kg body weight/day, about 1 ng/kg body weight/day to about 1 μg/kg body weight/day, about 1 ng/kg body weight/day to about 100 ng/kg body weight/day, about 1 ng/kg body weight/day to about 10 ng/kg body weight/day, about 10 ng/kg body weight/day to about 100 mg/kg body weight/day, about 10 ng/kg body weight/day to about 10 mg/kg body weight/day, about 10 ng/kg body weight/day to about 1 mg/kg body weight/day, about 10 ng/kg body weight/day to about 100 μg/kg body weight/day, about 10 ng/kg body weight/day to about 10 μg/kg body weight/day, about 10 ng/kg body weight/day to about 1 μg/kg body weight/day, 10 ng/kg body weight/day to about 100 ng/kg body weight/day, about 100 ng/kg body weight/day to about 100 mg/kg body weight/day, about 100 ng/kg body weight/day to about 10 mg/kg body weight/day, about 100 ng/kg body weight/day to about 1 mg/kg body weight/day, about 100 ng/kg body weight/day to about 100 μg/kg body weight/day, about 100 ng/kg body weight/day to about 10 μg/kg body weight/day, about 100 ng/kg body weight/day to about 1 μg/kg body weight/day, about 1 μg/kg body weight/day to about 100 mg/kg body weight/day, about 1 μg/kg body weight/day to about 10 mg/kg body weight/day, about 1 μg/kg body weight/day to about 1 mg/kg body weight/day, about 1 μg/kg body weight/day to about 100 μg/kg body weight/day, about 1 μg/kg body weight/day to about 10 g/kg body weight/day, about 10 μg/kg body weight/day to about 100 mg/kg body weight/day, about g/kg body weight/day to about 10 mg/kg body weight/day, about 10 μg/kg body weight/day to about 1 mg/kg body weight/day, about 10 μg/kg body weight/day to about 100 μg/kg body weight/day, about 100 μg/kg body weight/day to about 100 mg/kg body weight/day, about 100 μg/kg body weight/day to about 10 mg/kg body weight/day, about 100 μg/kg body weight/day to about 1 mg/kg body weight/day, about 1 mg/kg body weight/day to about 100 mg/kg body weight/day, about 1 mg/kg body weight/day to about 10 mg/kg body weight/day, about 10 mg/kg body weight/day to about 100 mg/kg body weight/day.

In other embodiments, the PD-L1 antibody and/or active agent is administered at a dose of 500 g to 20 g every three days, or 25 mg/kg body weight every three days.

In other embodiments, each PD-L1 antibody and/or active agent is administered in the range of about 10 ng to about 100 ng per individual administration, about 10 ng to about 1 g per individual administration, about 10 ng to about 10 g per individual administration, about 10 ng to about 100 g per individual administration, about 10 ng to about 1 mg per individual administration, about 10 ng to about 10 mg per individual administration, about 10 ng to about 100 mg per individual administration, about 10 ng to about 1000 mg per injection, about 10 ng to about 10,000 mg per individual administration, about 100 ng to about 1 g per individual administration, about 100 ng to about 10 g per individual administration, about 100 ng to about 100 g per individual administration, about 100 ng to about 1 mg per individual administration, about 100 ng to about 10 mg per individual administration, about 100 ng to about 100 mg per individual administration, about 100 ng to about 1000 mg per injection, about 100 ng to about 10,000 mg per individual administration, about 1 g to about 10 g per individual administration, about 1 g to about 100 g per individual administration, about 1 g to about 1 mg per individual administration, about 1 g to about 10 mg per individual administration, about 1 g to about 100 mg per individual administration, about 1 g to about 1000 mg per injection, about 1 g to about 10,000 mg per individual administration, about 10 g to about 100 g per individual administration, about 10 g to about 1 mg per individual administration, about g to about 10 mg per individual administration, about 10 g to about 100 mg per individual administration, about 10 g to about 1000 mg per injection, about 10 g to about 10,000 mg per individual administration, about 100 g to about 1 mg per individual administration, about 100 g to about 10 mg per individual administration, about 100 g to about 100 mg per individual administration, about 100 g to about 1000 mg per injection, about 100 g to about 10,000 mg per individual administration, about 1 mg to about 10 mg per individual administration, about 1 mg to about 100 mg per individual administration, about 1 mg to about 1000 mg per injection, about 1 mg to about 10,000 mg per individual administration, about 10 mg to about 100 mg per individual administration, about 10 mg to about 1000 mg per injection, about 10 mg to about 10,000 mg per individual administration, about 100 mg to about 1000 mg per injection, about 100 mg to about 10,000 mg per individual administration and about 1000 mg to about 10,000 mg per individual administration. The antibodies of the present disclosure may be administered daily, every 2, 3, 4, 5, 6 or 7 days, or every 1, 2, 3 or 4 weeks.

In other particular embodiments, the amount of each PD-L1 antibody or active agent may be administered at a dose of about 0.0006 mg/day, 0.001 mg/day, 0.003 mg/day, 0.006 mg/day, 0.01 mg/day, 0.03 mg/day, 0.06 mg/day, 0.1 mg/day, 0.3 mg/day, 0.6 mg/day, 1 mg/day, 3 mg/day, 6 mg/day, 10 mg/day, 30 mg/day, 60 mg/day, 100 mg/day, 300 mg/day, 600 mg/day, 1000 mg/day, 2000 mg/day, 5000 mg/day or 10,000 mg/day.

In certain embodiments, the coding sequences for the PD-L1 antibody and/or other active agent(s) are incorporated into a suitable expression vector (e.g., viral or non-viral vector) for expressing an effective amount of the PD-L1 antibody or other active agent in a subject in need of treatment in accordance with the above-described methods. In certain embodiments comprising administration of e.g., one or more recombinant AAV (rAAV) viruses, the pharmaceutical composition may comprise the rAAVs in an amount comprising at least 10¹⁰, at least 10¹¹, at least 10¹², at least 10¹³, or at least 10¹⁴ genome copies (GC) or recombinant viral particles per kg, or any range thereof. In certain embodiments, the pharmaceutical composition comprises an effective amount of the recombinant virus, such as rAAV, in an amount comprising at least 10¹⁰, at least 10¹¹, at least 10¹², at least 10¹³, at least 10¹⁴, at least 10¹⁵ genome copies or recombinant viral particles genome copies per subject, or any range thereof.

Dosages can be tested in one or several art-accepted animal models suitable for any particular cell proliferative disorder or immune-compromised disease state.

Delivery methodologies may also include the use of polycationic condensed DNA linked or unlinked to killed viruses, ligand linked DNA, liposomes, eukaryotic cell delivery vehicles cells, deposition of photopolymerized hydrogel materials, use of a handheld gene transfer particle gun, ionizing radiation, nucleic charge neutralization or fusion with cell membranes, particle mediated gene transfer and the like.

### VIII. Diagnostic Uses of the Antibodies

The antigen binding molecules, e.g., antibodies or the antigen binding fragment thereof, of the present invention may also be used to detect and/or measure human or cynomolgus PD-L1, or human or cynomolgus PD-L1 expressing cells in a sample, e.g., for diagnostic purposes. For example, an anti-PD-L1 antibody, or the antigen binding fragment thereof, may be used to diagnose a condition or disease characterized by aberrant expression (e.g., over-expression, under-expression, lack of expression, etc.) of PD-L1. Exemplary diagnostic assays for PD-L1, e.g., contacting a sample, obtained from a patient, with an antibody of the invention, wherein the antibody is labeled with a detectable label or reporter molecule. Alternatively, an unlabeled antibody can be used in diagnostic applications in combination with a secondary antibody which is itself detectably labeled. The detectable label or reporter molecule can be a radioisotope, such as ³H, ¹⁴C, ¹⁸F, ³²P, ³⁵S, or ¹²⁵I; a fluorescent or chemiluminescent moiety such as fluorescein isothiocyanate, or rhodamine; or an enzyme such as alkaline phosphatase, betagalactosidase, horseradish peroxidase, or luciferase. Specific exemplary assays that can be used to detect or measure PD-L1 in a sample include enzyme-linked immunosorbent assay (ELISA), radioimmunoassay (RIA), and fluorescence-activated cell sorting (FACS). Samples that can be used in PD-L1 diagnostic assays according to the present invention include any tissue or fluid sample obtainable from a patient which contains detectable quantities of PD-L1 protein, or fragments thereof, under normal or pathological conditions. Generally, levels of PD-L1 in a particular sample obtained from a healthy patient (e.g., a patient not afflicted with a disease or condition associated with abnormal PD-L1 levels or activity) will be measured to initially establish a baseline, or standard, level of PD-L1. This baseline level of PD-L1 can then be compared against the levels of PD-L1 measured in samples obtained from individuals suspected of having a PD-L1 related disease or condition.

Moreover, the anti-PD-L1 antibodies described herein can be used to purify human PD-L1 via immunoaffinity purification.

### IX. Kits

Any of the compositions described herein, e.g., the anti-PD-L1 antigen binding molecules of the present invention, and/or the additional therapeutic agent, may be comprised in a kit. In a non-limiting example, the kit comprises an antigen binding molecule, e.g., an antibody or antigen binding fragment thereof. In certain embodiments, the kit further includes an additional therapeutic agent described herein.

The kit may further include reagents or instructions for treating a disease or disorder. It may also include one or more buffers.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also generally contains a second, third or other additional container into which the additional components may be separately placed. The kits may also comprise a second container means for containing a sterile, pharmaceutically acceptable buffer and/or other diluent. However, various combinations of components may be comprised in a vial. The kits of the present invention also typically include a means for containing the compositions of the invention, e.g., the anti-PD-L1 antigen binding molecules and/or the additional therapeutic agent, and any other reagent containers in close confinement for commercial sale.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

The present invention is further illustrated by the following examples which should not be construed as limiting. The contents of all references, patents and published patent applications cited throughout this application, as well as the Figures and Tables are incorporated herein by reference.

### EXAMPLES

### Example 1: Generation of Rabbit Anti Human PD-L1 Monoclonal Antibodies

### Immunization of Rabbits

Two New Zealand White rabbits (4-6 weeks of age) were immunized by injecting human PD-L1 protein into the rabbits under an IACUC approved protocol by Capralogics. Briefly, the recombinant protein containing human PD-1 extracellular domain (ECD) and rabbit Fc (BonOpus) or D1 domain and rabbit Fc (Yurogen) was used to immunize the rabbits. Serum samples were taken prior to the first immunization and 14 days after the 2^{nd} immunization.

Serum titration was performed using antigen specific enzyme-linked immunoassay (ELISA) assay. Briefly, microplates were precoated with 25 μl of 1 μg/ml human PD-L1-His (Sino Biological, Cat #10084-H08H) or cynomolgus PD-L1-His protein (Sino Biological, Cat. 90251-C08H). Rabbit sera were diluted at 1:1000, followed by 7-point 3-fold serial dilution. The serially diluted sera were loaded in assay buffer. HRP-conjugated goat anti-rabbit IgG (H+L) antibodies (Jackson Immuno Research, Cat #111-036-045) were used at 1:5000 dilution for detection, with 100 μl/well TMB substrate for color development and 50 μl/well of stop solution (1N H₂SO₄).

Rabbits with high specific titers were then selected for euthanization to isolate spleen aseptically.

The sera from the immunized rabbits were also subject to the PD-1: PD-L1 binding competition ELISA (cELISA). Briefly, microplates were precoated with 1 μg/μl of the human PD-L1-mFc fusion protein (Sino Biological, Cat #10084-H05H) overnight at 4° C.; Plates were then blocked and added with 100 μl/well of the followings: (1) 0.5 μg/ml human PD-1-His-hFc-Biotin fusion protein (Sino Biological, Cat #10377-H03H-B) alone, (2) 0.5 μg/ml human PD-1-His-hFc-Biotin fusion protein mixed with diluted Imfinzi (Durvalumab) (starting at 6000 ng/ml, followed by 8-point serial dilution); or (3) 0.5 μg/ml human PD-1-His-hFc-Biotin fusion protein mixed with diluted rabbit serum (starting at 1:10 dilution, followed by 8-point serial dilution). HRP-conjugated Neutratavidin (ThermoFisher, Cat #31001) was used at 1:20,000 dilution for detection, with 100 μl/well TMB substrate for color development and 50 μl/well of stop solution (1N H₂SO₄).

The results (data not shown) of the cELISA show that the sera from the immunized rabbits can block PD-1: PD-L1 interaction. The commercially available anti-PD-L1 antibody (Durvalumab) was used as a positive control.

### Sorting of B-Cells

Spleen tissues from immunized rabbits were collected and homogenized. Splenocytes were either used immediately for sorting or snap frozen, stored under −196° C., and thawed later for more sorting later on. Biotinylated human PD-L1 D1-His-Avi (Yurogen), human PD-L1 ECD-His (Sino Biological, Cat #10084-H08H), human PD-L1 mouse Fc fusion protein (Sino Biological, Cat #10084-H05H), or Cynomolgus PD-L1 ECD-His (Sino Biological, Cat. 90251-C08H) were used for staining of rabbit B cells. If sorting antigens are not biotinylated, biotinylation will be performed. Briefly, 10 mM stock solution of Sulfo-NHS-LC-Biotin (ThermoFisher Cat #A39257) were prepared by dissolve 1 mg biotin in 180 μL deionized water on ice. Based on protein amount to be biotinylated, 20 times of molar ratio of biotin were added to protein solutions and the mixture were incubated on ice for 2 hours. Free biotin were removed by dialyzing biotinylated protein against 1×PBS, with at least 2 buffer changes with 3 hours minimum interval in Slide-A-Lyzer™ mini dialysis cup (3.5K MWCO, 0.1 mL) (ThermoFisher, Cat #69550). For each sorting, multiple vials of splenocytes from selected rabbit were thawed and cultured in B-cell culture media (RPMI-1640, 15% FBS, 1×HEPES, 1×2-ME (1-Mercaptoethanol), 1% Penicillin/Streptomycin) overnight before sorting. Also prepare 96-well B cell feeding plates one day before. Briefly, irradiated YC1 in B cell culture media with proprietary growth factor cocktail were dispensed into 96-well culture plates (120 μL/well). On the day of sorting, suspended and loosely attached splenocytes were collected by gently pipetting medium against the culturing surface of flask. The cells were then transferred to conical tube and spin at 400×g for 3 minutes. The cell pellets were washed with ice cold FACS buffer (1×PBS+0.5% BSA) twice. The biotinylated PD-L1 protein was added at 5 μg/ml (final concentration). The mixture was incubated at RT for 20 min. The staining mixture was then spun at 400×g for 3 min and the cells were resuspended in FACS buffer and transfer cells into 1.5 ml amber Eppendorf tube. NeutrAvidin-Dy594 (1:300 dilution, Invitrogen Cat #22842) and FITC-conjugated anti-rabbit IgM antibody (1:500 dilution, Novus, Cat #MB7173) were then added and the mixture were incubated at 4° C. for 15-30 min. The staining mixture was centrifuged at 400×g for 3 min. The cells were washed twice with ice-cold FACS buffer. The washed cell pellets were resuspended at ˜107 cells/ml in ice-cold 1×PBS+1% FBS. At least 10 minutes before sorting, 7-AAD (1 μg/ml, final concentration) were added for live/dead cell discrimination. Single 7-AAD-/IgM-/Dy594+ cell was sorted into each well of seeded 96-well plate. 96-well B cell culture plates with sorted B cells were cultured in rabbit B cell complete medium (Yurogen) at 37° C. with 5% CO2 for 9-12 days.

### Screening of Single B Cell Culture

On day 9 to day 12 post sorting, 15 μL of B cell culture supernatants were collected for antigen specific ELISA assay as described above. On day 10 to day 13 post sorting, the B cell culture plates were centrifuged at 400×g for 3 min. Supernatants from positive clones (OD greater than cutoff 0.3 or cutoff 0.9 from antigen specific ELISA) were selected and subject to PD-1:PD-L1 cELISA (described above). The supernatants from positive clones were also subject to cell binding assay. Briefly, MDA-MB-231 cells (known to express human PD-L1) were first added to 96-well plate (1×10⁵ cells per well). Fifty microliters (50 μl) of B cell culture (undiluted) supernatant samples were incubated with cells for 30 minutes on ice. After being washed with FACS buffer, 100 μl of 1 μg/ml of Alex Fluor 647-conjugated goat anti-rabbit IgG for B cell culture detection was added and incubated for 30 minutes on ice. Cells were resuspended with 100 μl of FACS buffer after two washes. One hundred microliters of 1:50 diluted 7-AAD for live/dead staining was added to the cell suspension, which was ready for FACS analysis (BD Accuri C6 plus or LSR II Flow Cytometer, HTS). Purified rabbit anti-hPD-L1, clone 8C6 (Yurogen) was used as positive control. Anti-HEL.hIgG1.FES or rabbit IgG format was used as isotype control.

Cells of cELISA+FACS+ clones were collected on day 10-day 13 post sorting and preserved in 100 μL DNA/RNA shield (Zymo, Cat #R1100-250), and transferred to 250 μL PCR tubes.

Cloning (or Sequencing) of V_{H} and V_{L} Encoding Gene

Total RNAs from selected clones were purified from cell pellets preserved in DNA/RNA shield using RNeasy Mini Kit (Zymo, Cat #R1051) following manufacturer's protocol. Thirty-six μl nuclease-free water (Ambion, cat #AM9937) were used to elute total RNA. Eleven μl total RNA from each clone were mixed with 1 μl oligo (dT)12-18 primer (Invitrogen, Cat #58862) and 1 μl dNTPs (10 mM, ThermoScientific, Cat #R0182) and then were heated at 65° C. for 5 min. Then for each clone mixture, 4 μl 5× FirstStrand buffer, 1 μl 100 mM DTT, 1 μl RNAaseOUT (Invitrogen, Cat #10777-019) and 1 μl SuperScript III reverse transcriptase (Invitrogen, Cat #18080-044) were added. The reverse transcription reaction was carried out at 50° C. 1 hr and 75° C. 15 min to inactive SuperScript III enzyme. After cDNA synthesis, VH and VL genes were amplified separately with V_{H} and VL variable region primer pairs using Go-Taq enzyme mix or Kapa HIFI HotStart enzyme. The VH PCR reaction contains 5ul cDNA cDNA template, 0.5 μl 5′ nest primer for V_{H} (2) (10 μm), 0.5 μl 3′ nest primer for VH (10 μm), 2.5 μl 10×PCR buffer, 1 μl of 50 mM MgCl₂, 0.2 μl Taq DNA Polymerase (ThermoFisher, Cat #10342053) and 15.5 μl nuclease-free water. And the VH PCR condition is 94° C. 3 min, followed by 40 cycles of 94° C. 30 sec, 55° C. 30 sec, 72° C. 45 sec, and one final extension step at 72° C. for 5 minutes. The VL PCR reaction contains 5 μl cDNA template, 0.75 μl 5′ nest primer for VL (10 μm), 0.75 μl 3′ nest primer for VL (10 μm), 2.5 μl 10×PCR buffer, 1 μl of 50 mM MgCl₂, 0.2 μl Platinum™ Taq DNA Polymerase (ThermoFisher, Cat #10966018) and 15 μl nuclease-free water. And the VL PCR condition is 98° C. 3 min, followed by 40 cycles of 95° C. 15 sec, 64° C. 15 sec, 72° C. 30 sec, and one final extension step at 72° C. for 5 minutes. The VH and VL PCR products were separated on 1% agarose gel electrophoresis system. The expected size of VH and VL amplicon is ˜500 bp. The corresponding bands of VH and VL for each clone were cut from gel and VH and VL were extracted from gel with NucleoSpin® Gel and PCR Clean-Up Kit (Macherey-Nagel, Cat #740609.250) following manufacturer's protocol. Twelve −30 μl of elution buffer were used to eluted VH and VL PCR products depending on the amount of PCR products. The linear expression module cassette (LEM) PCR products were constructed by overlapping PCR with the C fragment containing CMV promotor, VH or VL, and the H fragment containing rabbit IgG heavy-chain CH1 fragment, human IgG1 C_{H}2 and C_{H}3 fragment, or light-chain constant region followed by SV40 transcription terminator and poly A signal sequences. LEM heavy chain PCR reaction contains 20 ng (1-5 μl) VH fragment, 1 μl CH_RK fragment (10 ng/μl), 1 μl HH_RK_hIgG1Fc fragment (10 ng/μl), 1 μl 5′ primer C-frag 3 (10 μM), 1 μl 3′ primer H-frag 4 (10 μM), 10 μl 5× LongAmp Taq Reaction Buffer, 1.5 μl dNTPs (10 mM), 2 μl LongAmp Taq DNA Polymerase (NewEngland BioLabs, Cat #E5200S) and 31.5 μl Nuclease-free water. LEM light chain PCR reaction contains 20 ng (1-5 μl) VL fragment, 1 μl CL_RK fragment (10 ng/μl), 1 μl HL_RK fragment (10 ng/μl), 1 μl 5′ primer C-frag 3 (10 μM), 1 μl 3′ primer H-frag 4 (10 μM), 10 μl 5× LongAmp Taq Reaction Buffer, 1.5 μl dNTPs (10 mM), 2 μl LongAmp Taq DNA Polymerase and 31.5 μl Nuclease-free water. The PCR cycle condition is 94° C., 3 min, 35 cycles of 94° C. 30 sec; 55° C. 40 sec; 68° C. 3 min 30 sec, and one final of 68° C. 5 min. Five μl of PCR product were used to check the size and magnitude of amplification by 0.8% agarose gel electrophoresis (shown below). The remaining PCR products were purified with NucleoSpin® Gel and PCR Clean-Up Kit following manufacturer's protocol. Thirty-two μl of elution buffer were used to eluted LEM PCR products. Sequencing was performed for LEM constructs by Quintara Bioscience (Quintara, Cambridge, MA).

### Screening of LEM Transfection

Cell binding assay (as described above) was performed on the supernatant samples obtained from LEM transfection. Briefly, fifty microliters (50 μl) LEM transfection supernatant samples (undiluted or diluted at 1:5, 1:10 or 1:50) was incubated with cells for 30 minutes on ice. After being washed with FACS buffer, 100 μl of 1 μg/ml of Alex Fluor 647-conjugated anti-human IgG was added and incubated for 30 minutes on ice. Cells were resuspended with 100 μl of FACS buffer after two washes. One hundred microliters of 1:50 diluted 7-AAD for live/dead staining was added to the cell suspension, which was ready for FACS analysis (BD Accuri C6 plus or LSR II Flow Cytometer, HTS). Durvaluimab was used as positive control LEM transfection supernatant samples, respectively. Anti-HEL.hIgG1.FES or rabbit IgG format (Biolntron) was used as isotype control.

### Example 2: Cloning and Expression of Genes Encoding Antibody Variable Regions of Anti-PD-L1 Antibodies

The genes encoding V_{H} and V_{L} of various clones were then cloned into Yurogen's expression vectors consisting human IgG1 Fc and rabbit CH1. After sequence confirmation, IgG heavy and light chain plasmids were used for small scale antibody production in HEK293T cells. Briefly, ˜5×10⁶ HEK293T cells were seeded to 12-well plate one day before transfection. For each clone, dilute 500 ng Heavy chain LEM PCR products and 500 ng Light chain LEM PCR products with 45 μL Opti-MEM medium (Gibco, Cat #31985-070). Dilute 3 μL Lipofectamine 2000 Reagent (Invitrogen, Cat #11668019) with 47 μL Opti-MEM medium. Mix diluted LEM PCR products and Lipofectamine, incubate it at RT for 10 minutes. Replace HEK293T medium with 1 mL of Opti-MEM medium. Add the mixed solution to each well dropwise. Swirl the plate gently to mix transfecting reagent and medium well. Transfected cells were incubated at 37° C., 5% CO₂ for 48-72 hours, then harvested supernatant for further screening.

### Example 3: Specific Binding of Anti-PD-L1 Antibodies to PD-L1

### Binding Specificity

Purified antibodies were tested by FACS to confirm their binding and affinity against human and cynomolgus PD-L1-His recombinant protein. Briefly, Maxisorp plates (Nunc) were coated with 100 ul/well of 0.5 μg/ml human PD-L1-His (Sino Biological, Cat #10084-H08H) or cynomolgus PD-L1-His (Sino Biological, Cat. 90251-C08H) protein diluted in 1×PBS (GIBCO) overnight at 4° C. Plates were washed three times with 300 μl/well washing buffer (1×PBST, diluted from 20×PBST (Thermo Scientific) with distilled water)) and then blocked with 300 μl/well of blocking buffer (1×PBST/I % BSA) for 2 hours at RT on shaker (100 rpm). Plates were washed and added with 100 μl/well of testing antibodies, which were serial diluted with assay buffer (PBST/1% BSA). The antibodies were incubated at room temperature for 2 hours on plate shaker at 100 rpm. Plates were washed. One hundred microliter of horseradish peroxidase (HRP) conjugated detection antibody was added into each well and incubated at room temperature for 1 hour on plate shaker at 100 rpm. Plates were washed and added with 100 μl/well TMB substrate for color development at room temperature in dark for up to 10 minutes. After addition of 50 μl/well of stop solution (1N H₂SO₄), plates were read at 450 nm on a plate reader (SpectraMax M5).

Table 21 below shows that the exemplary antibodies of the invention specifically bind to both human and cynomolgus PD-L1. Durvalumab (AstraZeneca) was used as positive control. Anti-HEL isotype control (Biolntron) was used as negative control.

TABLE 21
Binding EC50 (nM) of anti-PD-L1 antibodies
to human and cynomolgus PD-L1protein

| Antibody | human PD-L1 | Cyno PD-L1 |
|---|---|---|
| 27A2 | 0.0146 | 0.0113 |
| 28B3 | 0.0159 | 0.0177 |
| 32 E2 | 0.1062 | 0.1787 |
| 33 E8 | 0.0399 | 0.0414 |
| 33H7 | 0.0171 | 0.0130 |
| 55H3/76D6 | 0.0199 | 0.0126 |
| 56 E5 | 0.0184 | 0.0078 |
| 56H3 | 0.0172 | 0.0076 |
| 57B10 | 0.0186 | 0.0071 |
| 57F11 | 0.0216 | 0.0138 |
| 62 E6 | 0.0240 | 0.0188 |
| 69D10 | 0.0215 | 0.0177 |
| 73C8 | 0.0160 | 0.0121 |
| 74A9 | 0.0229 | 0.0198 |
| 74F4 | 0.0157 | 0.0125 |
| Durvalumab | 0.0158 | 0.0081 |
| Anti-HEL | n.a. | n.a. |

Similar assays were performed to test whether the exemplary antibodies of the present disclosure bind to mouse PD-L1 (Sino Biological, Cat #50010-M08H), rat PD-L1 (Sino Biological, Cat #80450-R08H), canine PD-L1 (Acro Biosystems, Cat #PDL-C52H4) or rabbit PD-L1 (Acro Biosystems, Cat #PDL-R52H6). No binding of the exemplary antibodies of the present disclosure to mouse, rat, canine or rabbit PD-L1 has been detected.

Similar assays were performed to test whether the exemplary antibodies of the present disclosure bind to other proteins that belong to the same family of PD-L1. The exemplary antibodies of the present disclosure showed no binding to CD80 (Sino Biological, Cat #10698-H08H), CD86 (Sino Biological, Cat #10699-H08H), BH-H2 (Sino Biological, Cat #11559-H08H), B7-H3 (Sino Biological, Cat #11188-H08H) and PD-L2 (Sino Biological, Cat #10292-H08H).

### Binding Affinity

The affinity of the binding of the exemplary anti-PD-L1 antibodies of the present disclosure was determined. Assay was performed according to the protocol recommended by Gator or Gator Plus (Gator Bio Inc.). Briefly, human PD-L1-His (Sino Biological, Cat #10084-H08H) was serially diluted 4-fold, starting at 4 μg/ml. After three times of regeneration in generation buffer (Gator Bio, Cat #120012) and neutralization of HFC probe (Gator Bio, Cat #160003), 1 μg/ml of antibody to be tested in Q buffer (Gator Bio, Cat #120010) was immobilized onto HFC probe. After a baseline step, probe immobilized with antibody was dipped into antigen solutions. The association and dissociation of antigen from antibody was measured on BLI machine Gator. Affinity was calculated using Gator Bio analysis software.

Table 22 below summarizes the binding affinity to human PD-L1 using Gator. Durvalumab (AstraZeneca) was used as positive control. Anti-HEL isotype control (BioIntron) was used as negative control.

TABLE 22
Binding affinity of anti-PD-L1 antibodies to human PD-L1

| Antibody | Kon (×10⁵, 1/Ms) | Koff (×10⁻⁴, 1/s) | K_{D} (nM) |
|---|---|---|---|
| 27A2 | 6.43 | 0.36 | 0.06 |
| 28B3 | 8.10 | 0.76 | 0.09 |
| 32 E2 | 3.71 | 1.13 | 0.31 |
| 33 E8 | 3.61 | 4.18 | 1.16 |
| 33H7 | 5.36 | 3.29 | 0.61 |
| 55H3/76D6 | 4.98 | 3.17 | 0.64 |
| 56 E5 | 7.48 | 5.60 | 0.75 |
| 56H3 | 6.86 | 3.99 | 0.58 |
| 57B10 | 8.10 | 0.77 | 0.09 |
| 57F11 | 3.71 | 1.13 | 0.31 |
| 62 E6 | 7.88 | 3.42 | 0.43 |
| 69D10 | 7.18 | 65.3 | 9.09 |
| 73C8 | 8.04 | 0.939 | 0.12 |
| 74A9 | 6.43 | 2.34 | 0.36 |
| 74F4 | 6.48 | 2.38 | 0.36 |
| Durvalumab | 7.27 | 2.56 | 0.35 |

TABLE 23
Binding affinity of anti-PD-L1 antibodies to cynomolgus PD-L1

| Antibody | Kon (×10⁵, 1/Ms) | Koff (×10⁻⁴, 1/s) | K_{D} (nM) |
|---|---|---|---|
| 27A2 | 3.90 | 0.13 | 0.03 |
| 28B3 | 4.03 | 1.32 | 0.33 |
| 32 E2 | 1.33 | 0.70 | 0.53 |
| 33 E8 | 2.18 | 2.02 | 0.93 |
| 33H7 | 3.50 | 0.81 | 0.23 |
| 55H3/76D6 | 3.07 | 0.11 | 0.03 |
| 56 E5 | 3.13 | 0.10 | 0.03 |
| 56H3 | 3.49 | 0.87 | 0.25 |
| 57B10 | 2.84 | 0.07 | 0.03 |
| 57F11 | 3.73 | 1.01 | 0.27 |
| 62 E6 | 3.13 | 0.74 | 0.02 |
| 69D10 | 4.28 | 0.77 | 0.18 |
| 73C8 | 3.44 | 0.10 | 0.03 |
| 74A9 | 2.96 | 9.15 | 3.09 |
| 74F4 | 3.87 | 0.05 | 0.01 |
| Durvalumab* | 3.64/5.11 | 2.01/1.85 | 0.552/0.36 |

Tested in duplicate

### Example 4: Specific Binding of Anti-PD-L1 Antibodies to PD-L1 Expressing Cells

The assay described in this example is to determine whether the anti-PD-L1 antibodies of the present disclosure specifically binds to PD-L1 expressing cells.

In this assay, MDA-MB-231-GFP cells (known to express human PD-L1) were collected from fresh culture. Cells were then resuspended in FACS buffer (1×DPBS/1% FBS) at 10⁶/ml concentration. One hundred microliter (100 μl) of cells were aliquoted into a single well in 96 well plates. The antibodies to be tested were serially diluted at 1:3 fold starting at 15 ug/mL and were then added into each well. The antibodies were mixed with cells and the mixtures were incubated on ice for 1 hour. After washing with FACS buffer, one hundred microliter (100 kl) of diluted PE-conjugated anti-human Fc secondary antibody (Jackson Immuno Research, Cat #109-116-098) was added into each well and incubated for 30 minutes on ice. After washing with FACS buffer, stained cells were resuspended at diluted 7-ADD (Biolegend, Cat #420404, used at 1:100 dilution) and analyzed on Guava 11HT (Luminex). Data was analyzed using Flowjo software.

Table 24 below shows that the exemplary antibodies of the present disclosure specifically bind to PD-L1 expressed on MDA-MD-231-GFP cell surface. Durvalumab (AstraZeneca) was used as positive control. Anti-HEL isotype control (Biolntron) was used as negative control.

TABLE 24
Binding of anti-PD-L1 antibodies to MDA-MB-231-GFP cells

| Antibody | EC₅₀ (nM) |
|---|---|
| 27A2 | 0.14 |
| 28B3 | 0.10 |
| 32 E2 | 18.51 |
| 33 E8 | 1.19 |
| 33H7 | 0.34 |
| 55H3/76D6 | 2.93 |
| 56 E5 | 1.29 |
| 56H3 | 0.19 |
| 57B10 | 4.82 |
| 57F11 | 1.24 |
| 62 E6 | 2.03 |
| 69D10 | 2.0 |
| 73C8 | 1.81 |
| 74A9 | 0.34 |
| 74F4 | 1.44 |
| Durvalumab | 0.10 |
| Isotype control | n.a. |

### Example 5: Blockade of PD-1/PD-L1 Interaction by Anti-PD-L1 Antibodies

The assays in this example are to determine whether the anti-PD-L1 antibodies of the present disclosure can block the PD-1/PD-L1 interaction.

cELISA Assay

Competition ELISA (cELISA) assay was used to determine the blockade of PD-1/PD-L1 interaction by the exemplary anti-PD-L1 antibodies of the present disclosure. Briefly, microplates were precoated with 1 μg/μl of human PD-L1-mFc fusion protein (Sino Biological, Cat #10084-H05H) diluted in 1×PBS (GIBCO) and incubated overnight at 4° C.; Plates were washed four times with 300 μl/well washing buffer (1×PBST, diluted from 20×PBST (Thermo Scientific) with distilled water)) and then blocked with 300 μl/well of blocking buffer (1×PBST/3% BSA) for 2 hours at RT on shaker (100 rpm). Plates were washed and added with 100 μl/well of the followings: (1) 1 μg/ml human PD-1-His-hFc-Biotin fusion protein (Sino Biological, Cat #10377-H03H-B) alone, (2) human PD-1-His-hFc-Biotin fusion protein mixed with diluted Bavencio ((Avelumab) or Imfinzi (Durvalumab) or anti-HEL isotype control antibody (Biolntron) starting at 50 nM, followed by 12-point 1:2 fold serial dilution; or (3) human PD-1-His-hFc-Biotin fusion protein mixed with diluted test anti-PD-L1 antibody starting at 50 nM, followed by 12-point 1:2 fold serial dilution. One hundred microliter (100 μl)/well of 1:20,000 diluted HRP-conjugated Avidin (Invitrogen, Cat #A2664) was added into each well and incubated at room temperature for 1 hour on plate shaker at 100 rpm. Plates were washed and added with 100 μl/well TMB substrate for color development at room temperature in dark for up to 10 minutes. After addition of 50 μl/well of stop solution (1N H₂SO₄), plates were read at 450 nm on a plate reader (SpectraMax M5).

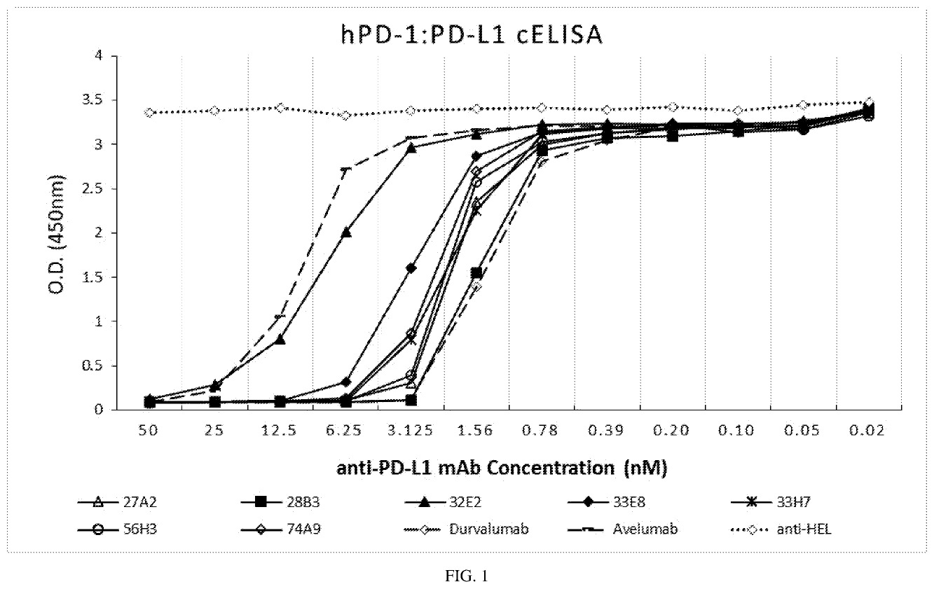

Table 25 below summarizes the IC₅₀ value of the exemplary antibodies of the present disclosure for blocking the interaction between PD-1 and PD-L1. Imfinzi (Durvalumab) and Bavencio (Avelumab), both commercially available anti-PD-L1 antibodies were used as positive controls. As shown in Table 25 and FIG. 1, exemplary anti-PD-L1 antibodies of the present disclosure can effectively block the interaction between PD-1 and PD-L1.

TABLE 25
IC₅₀ Value of Blockade of PD-1/PD-L1
Interaction by anti-PD-L1 Antibodies

| Antibody | IC₅₀ (nM) |
|---|---|
| 27A2 | 1.9 |
| 28B3 | 1.5 |
| 32E2 | 7.5 |
| 33E8 | 3.1 |
| 33H7 | 2.1 |
| 56H3 | 2.1 |
| 74A9 | 2.3 |
| Durvalumab | 1.4* |
| Avelumab | 11.2* |

*Average of triplicate

### Promega Assay

This assay is to test whether the exemplary antibodies of the present invention can block the interaction between PD1 and cell surface PD-L1 using PD1/PD-L1 blockade assay kit (Promega, Cat. J1255), which can also be used to test antibody potency for blocking of PD1/PD-L1 interaction. The assay was performed according to the manufacturer's manual. Briefly, one hundred microliters (100 μl) of CHO-K1-hPD-L1 cell suspension in F-12 medium with 10% FBS was added to a single well of cell culture treated flat bottom white 96 plate and cultured at 37° C. in a CO2 incubator for 16 hours. The antibodies to be tested, or positive control commercially available anti-PD-L1 antibody Imfinzi (Durvalumab), Bavencio (Avelumab), Atezolizumab analogue (BioIntron), or SGN-PD-L1 analogue anti-PD-L1 antibody (BioIntron) (or anti-HEL isotype control antibody were serially diluted using 1×RPMI/1% FBS. Subsequently, forty microliters (40 μl) of each diluted antibody were added into each well, in which the overnight culture medium was removed, followed by the addition of 40 μl of effector PD-1 cell suspension into each well. Plate was incubated at 37° C. in a CO₂ incubator for 6 hours. Then, eighty microliters (80 μl) of BIO-Glo reagent were added to each well and the plate is proceeded immediately for measurement of luminescence signal on a plate reader (SpectraMax M5).

Table 26 below summarizes the IC₅₀ value of the exemplary antibodies of the present disclosure for blocking the interaction between PD-1 and cell surface PD-L1. Durvalumab was used as positive control. As shown in Table 27, exemplary anti-PD-L1 antibodies of the present disclosure can effectively block the interaction between PD-1 and cell surface PD-L1.

TABLE 26
IC₅₀ Value of Blockade of PD-1/PD-L1
Interaction by anti-PD-L1 Antibodies

| Antibody | IC₅₀ (nM) |
|---|---|
| 27A2 | 0.23 |
| 28B3 | 0.24 |
| 33H7 | 0.52 |
| 55H3/76D6 | 0.97 |
| 56E5 | 0.41 |
| 56H3 | 0.36 |
| 57B10 | 0.47 |
| 62E6 | 0.41 |
| 74A9 | 0.29 |
| Durvalumab | 0.14* |

*Average of triplicate

Table 27 below summarizes the IC₅₀ value of the exemplary antibodies of the present disclosure for blocking the interaction between PD-1 and cell surface PD-L1 in another assay. Durvalumab, Avelumab, Atezolizumab analogue, SGN-PD-L analogue were used as positive control. All antibodies were tested in duplicate except that Avelumab was tested in pentaplicate. As shown in Table 27, exemplary anti-PD-L1 antibodies of the present disclosure can effectively block the interaction between PD-1 and cell surface PD-L1.

TABLE 27
IC₅₀ Value of Blockade of PD-1/PD-L1
Interaction by anti-PD-L1 Antibodies

| Antibody | IC₅₀ (nM) |
|---|---|
| 27A2 | 0.35 |
| 28B3 | 0.33 |
| 33E8 | 0.38 |
| 74A9 | 0.46 |
| SGN-PDL1* | 0.68 |
| Atezolizumab* | 0.33 |
| Durvalumab | 0.43 |
| Avelumab | 0.49** |

*Analogue antibody
**Average of pentaplicate

### Example 6: ADCC of the Anti-PD-L1 Antibodies

Antibody-dependent cellular cytotoxicity (ADCC) of the exemplary anti-PD-L1 antibodies of the present disclosure was measured in this experiment.

### InvivoGen Assay Using PD-L1-Expressing Raji Cells

NFAT-CD16 Lucia Luciferase Reporter Jurkat cells and PD-L1-expressing Raji cells were purchased from InvivoGen and expanded for assay set-up. First, twenty microliters (20 μl) of antibodies to be tested were added into a single well of a flat-bottom 96-well cell culture plate. Subsequently, ninety microliters (90 μl) of Raji-PD-L1 cells (˜100,000 cells) were added to each well. After incubation of the plate at 37° C. in a CO₂ incubator for 1 hour, ninety microliters (90 μl) of Jurkat-Lucia™-NFAT-CD16 cells (˜200,000 cells) were added into each well, and the plate was incubated at 37° C. in a CO₂ incubator for 6 hours. Twenty microliters (20 μl) of co-incubated Raji-PD-L1 and Jurkat-Lucia™ NFAT CD16 cell culture supernatant from each well was transferred into a single well in a 96-well white (opaque, Corning), followed by addition of 50 μl of QUANTI-Luc™ substrate (InvivoGen, prepared in advance following vendor's instruction)) into each well. Plates were proceeded immediately for measurement of luminescence signal on a plate reader (SpectraMax M5). Anti-HEL isotype control antibody was used as a negative control.

Table 28 below summarizes the EC₅₀ of the ADCC of the exemplary anti-PD-L1 antibodies of the present disclosure.

TABLE 28
EC₅₀ Value of the ADCC of anti-PD-L1 antibodies

| Antibody | EC₅₀ (nM) |
|---|---|
| 28B3 | 4.2 |
| 32 E2 | 4.0 |
| 33 E8 | 3.6 |
| 55H3/76D6 | 3.1 |
| 56 E5 | 1.9 |
| 57B10 | 3.7 |
| 69D10 | 2.2 |
| 73C8 | 1.0 |
| 74A9 | 2.3 |
| 74F4 | 1.0 |
| Anti-HEL | n.a. |

### InvivoGen Assay Using MDA-MB-231 GFP Cells

This assay is similar to the InvivoGen assay as described above except that the Raji-PD-L1 cells were replaced by MDA-MB-231 GFP cells. Avelumab and Durvalumab were used as a positive control. Anti-HEL isotype control antibody was used as a negative control. Table 29 below summarizes the EC₅₀ value of ADCC of the exemplary anti-PD-L1 antibodies of the present disclosure.

TABLE 29
EC₅₀ Value of the ADCC of anti-PD-L1 antibodies

| Antibody | EC₅₀ (nM) |
|---|---|
| 27A2 | 2.3 |
| 28B3 | 0.8 |
| 32 E2 | 1.8 |
| 33 E8 | 0.5 |
| 33H7 | 0.1 |
| 55H3/76D6 | 0.5 |
| 56 E5 | 0.2 |
| 56H3 | 0.2 |
| 57B10 | 0.5 |
| 57F11 | 0.2 |
| 62 E6 | 0.1 |
| 69D10 | 1.0 |
| 73C8 | 0.2 |
| 74A9 | 0.1 |
| Avelumab* | 0.4 |
| Anti-HEL* | n.a. |

*Average of duplicate

### Example 7: Internalization of Anti-PD-L1 Antibodies and PD-L1

This example is to measure the internalization of the complex formed by the exemplary anti-PD-L1 antibodies of the present disclosure and PD-L1 express on cell surface.

### FACS Based Internalization Assay

About one hundred thousand (˜10⁵) MDB-MA-231 cells were incubated with antibodies to be tested at indicated concentration (2, 1, 0.5 and 0.25 μg/ml) on ice. Commercial ADC drug Trodelvy (Sacituzumab govitecan-hziy, Immunomedics) were used as positive controls. Commercial anti-PD-L1 antibodies Durvalumab and Avelumab were also tested for internalization. One hour after incubation, two aliquots were taken out and transferred into 37° C. incubator for 2 or 4 hours of incubation, respectively. Samples collected at different time points were washed with ice cold FACS buffer for two times and then stained with PE-conjugated anti-human Fc secondary antibody (Jackson Immuno Research) for 1 hour on ice. After two washes with ice cold FACS buffer, cells were stained with 7-AAD (Biolegend) and then fixed with 2% of paraformaldehyde (diluted from 10% paraformaldehyde (MilliporeSigma, Cat #R04586)). MDB-MA-231 cells were known to express both PD-L1 and Trop2. Sacituzumab was known to be an anti-Trop2 antibody that induces efficient internalization upon binding to Trop2 on cell surface.

Samples were then analyzed on Guava 11HT (Luminex) and data analysis was done with Flowjo software. Internalization rate was calculated as:

$$\% of internalization = \{ [ ( MFI of sample on ice ) -$$

$$( MFI of sample of indicated incubation time at 37 ° C . ) ] /$$

$$( MFI of sample on ice ) \} \times 100$$

Table 30 below summarizes the internationalization of the complex formed by different anti-PD-L1 antibodies and PD-L1 expressed on MDA-MB-231 cell surface. The results show that the exemplary anti-PD-L1 antibodies induce internalization upon binding to cell surface PD-L1.

TABLE 30
Internalization induced by anti-PD-L1 antibodies

| Internalization | | Time at | | | | | | 55H3/ | | | |
|---|---|---|---|---|---|---|---|---|---|---|---|
| (%) | | 37° C. | 27A2 | 28B3 | 32E2 | 33E8 | 33H7 | 76D6 | 56E5 | Durvalumab | Avelumab |
| 2 | μg/ml | 0 h | 0.0 | 0.0 | 0.0 | 0.0 | 0.0 | 0.0 | 0.0 | 0.0 | 0.0 |
| 2 | μg/ml | 2 h | 12.7 | 8.3 | 2.4 | 29.6 | 17.8 | 24.2 | 24.7 | 6.2 | 11.5 |
| 2 | μg/ml | 4 h | 9.7 | 6.0 | 7.6 | 30.2 | 12.6 | 22.2 | 26.5 | 9.4 | 12.9 |
| 1 | μg/ml | 0 h | 0.0 | 0.0 | 0.0 | 0.0 | 0.0 | 0.0 | 0.0 | 0.0 | 0.0 |
| 1 | μg/ml | 2 h | 13.7 | 15.3 | 1.1 | 20.4 | 14.4 | 22.1 | 23.8 | 12.2 | 3.5 |
| 1 | μg/ml | 4 h | 13.7 | 15.5 | 7.2 | 23.3 | 12.4 | 17.9 | 20.6 | 13.5 | 5.2 |
| 0.5 | μg/ml | 0 h | 0.0 | 0.0 | 0.0 | 0.0 | 0.0 | 0.0 | 0.0 | 0.0 | 0.0 |
| 0.5 | μg/ml | 2 h | 15.5 | 12.0 | 0.4 | 22.8 | 17.0 | 20.8 | 20.7 | 20.4 | 7.2 |
| 0.5 | μg/ml | 4 h | 14.7 | 19.9 | 5.6 | 25.8 | 14.2 | 19.9 | 19.8 | 8.8 | 9.7 |
| 0.25 | μg/ml | 0 h | 0.0 | 0.0 | 0.0 | 0.0 | 0.0 | 0.0 | 0.0 | 0.0 | 0.0 |
| 0.25 | μg/ml | 2 h | 18.5 | 20.4 | 2.2 | 20.9 | 15.2 | 22.9 | 22.9 | 15.5 | 12.8 |
| 0.25 | μg/ml | 4 h | 18.3 | 17.6 | −25.9 | 21.6 | 12.3 | 20.6 | 22.1 | 12.7 | 15.3 |

| Internalization | | Time at | | | | | | | | | | anti-HEL |
|---|---|---|---|---|---|---|---|---|---|---|---|---|
| (%) | | 37° C. | 56H3 | 57B10 | 57F11 | 62 E6 | 69D10 | 73C8 | 74A9 | 74F4 | Trodelvy | isotype |
| 2 | μg/ml | 0 h | 0.0 | 0.0 | 0.0 | 0.0 | 0.0 | 0.0 | 0.0 | 0.0 | 0.0 | 0.0 |
| 2 | μg/ml | 2 h | 6.5 | 19.2 | 14.5 | 16.1 | 17.7 | 22.5 | 13.9 | 24.1 | 36.2 | NA |
| 2 | μg/ml | 4 h | 4.8 | 22.1 | 15.4 | 17.9 | 21.2 | 21.7 | 11.7 | 22.0 | 46.1 | NA |
| 1 | μg/ml | 0 h | 0.0 | 0.0 | 0.0 | 0.0 | 0.0 | 0.0 | 0.0 | 0.0 | 0.0 | 0.0 |
| 1 | μg/ml | 2 h | 15.3 | 12.7 | 16.7 | 16.4 | 15.9 | 19.5 | 17.7 | 19.5 | 36.5 | NA |
| 1 | μg/ml | 4 h | 9.1 | 15.6 | 16.8 | 17.7 | 18.2 | 15.7 | 9.2 | 18.2 | 44.5 | NA |
| 0.5 | μg/ml | 0 h | 0.0 | 0.0 | 0.0 | 0.0 | 0.0 | 0.0 | 0.0 | 0.0 | 0.0 | 0.0 |
| 0.5 | μg/ml | 2 h | 16.1 | 11.1 | 19.7 | 12.2 | 16.2 | 17.8 | 13.2 | 22.5 | 38.0 | NA |
| 0.5 | μg/ml | 4 h | 19.0 | 21.7 | 14.7 | 24.0 | 18.0 | 23.0 | 12.0 | 15.7 | 45.6 | NA |
| 0.25 | μg/ml | 0 h | 0.0 | 0.0 | 0.0 | 0.0 | 0.0 | 0.0 | 0.0 | 0.0 | 0.0 | 0.0 |
| 0.25 | μg/ml | 2 h | 16.3 | 19.3 | 17.9 | 22.3 | 18.2 | 22.0 | 11.5 | 19.0 | 35.7 | NA |
| 0.25 | μg/ml | 4 h | 16.0 | 16.5 | 21.3 | 21.0 | 15.9 | 15.6 | 11.0 | 15.3 | 45.1 | NA |

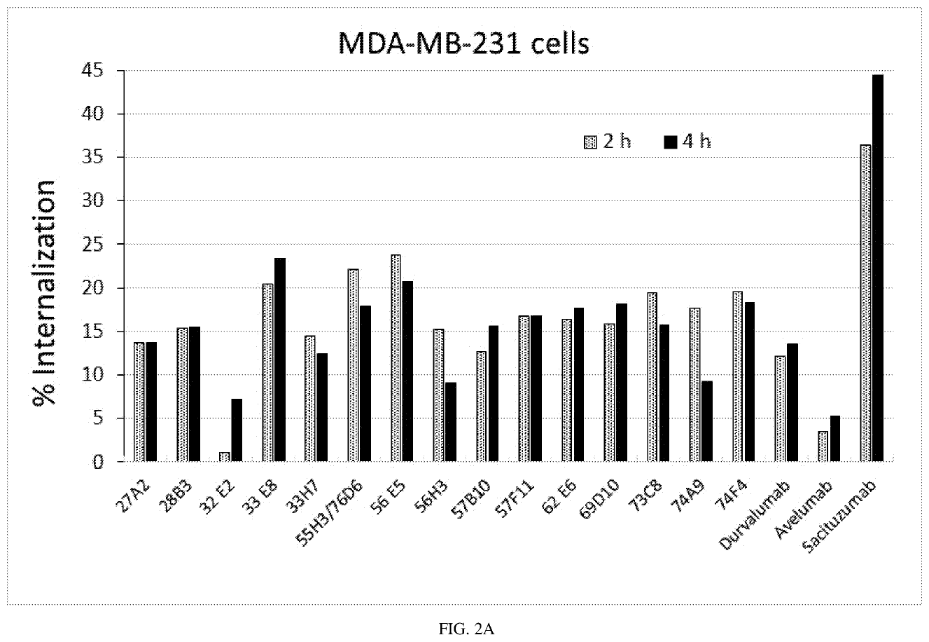

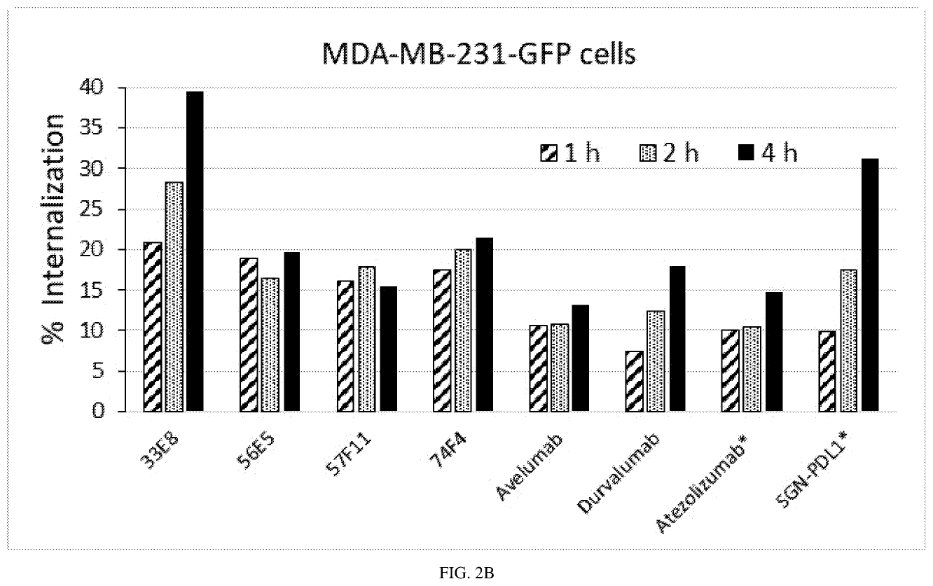

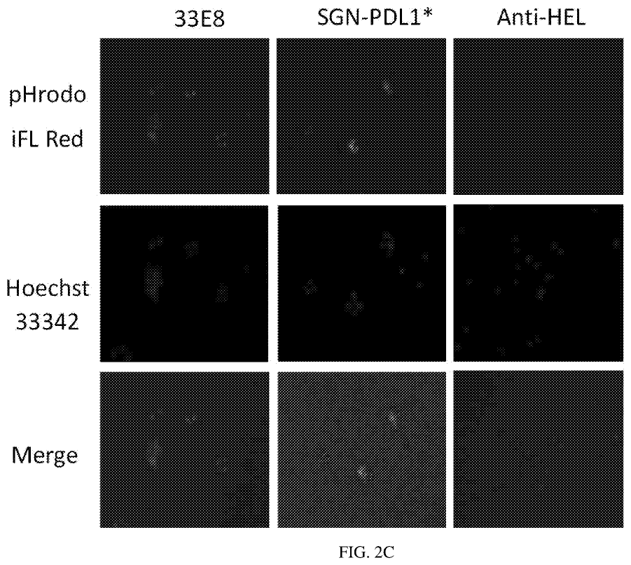

FIG. 2A shows that incubation of MDA-MB-231 cells for 4 hours at 37° C. with 1 μg/ml of 33E8 induces highest level of internalization (shown as percentage of internalization), compared to all other anti-PD-L1 antibodies including Durvalumab. Internalization level induced by 32E2 or Avelumab is minimum. FIG. 2B shows that incubation of MDA-MB-231-GFP cells with 1 μg/ml 33E8 for 1 hour, 2 hours, and 4 hours induced much higher level (shown as percentage of internalization) of internalization than any other anti-PD-L1 antibodies including Avelumab, Durvelumab, Atezolizuab analogue (Biolntron), and SGN-PDL1 analogue (Biolntron).

### Dye Based Internalization Assay

The Invitrogen™ Zenon™ pHrodo™ iFL RED human IgG Labeling Reagents (Cat #Z25612) was used to determine the internalizing properties of the exemplary anti-PD-L1 antibodies following manufacture's instruction. The internalization of antibodies coupled with the Zenon™ pHrodo™ iFL RED human IgG Labeling reagents increase the fluorescence signal at low pH intracellular compartment in a cell, providing a method for visualizing internalization.

Briefly, 4× working solutions for each antibody (24 μg/ml) were prepared in complete DMEM medium (10% FBS, 1× Pen/Strep). Twenty five microliter (25 μL) of 4× antibody working solution was aliquoted to each well of a clear-bottom 96-well plate. Twenty five microliter (25 μL) of 4× Zenon working solutions were then added to wells containing antibodies. The mixture of antibody and Zenon solution was incubated for 5 minutes at room temperature to allow the labeling complexes to form, followed by addition of 50 μL of 2×10⁶/mL human PD-L1-expressing Raji cells in cell culture medium. Cells were then incubated with the labeling complex under standard cell culture conditions. After 4 to 24 hours of incubation, cells were analyzed under immunofluorescent microscope (Invitrogen EVOS cell imaging system M5000) to visualize the internalization of antibodies by cells. As shown in FIG. 2C, induction of internalization could be visualized in the representative pictures, which showed that internalization can be induced by 33E8 and SGN-PDL1 analogue antibodies, but not anti-HEL isotype control antibody. Hoechst 33342 (Invitrogen, Cat. H3570) is a popular cell-permeant nuclear counterstain that emits blue fluorescence, and is used to distinguish condensed nuclei inside the cell.

### Example 8: Cytotoxicity of Anti-PD-L1 Antibodies Conjugated to Cytotoxic Agent

This study examines whether the exemplary antibodies of the present disclosure, conjugated to a cytotoxic agent, with can induce killing PD-L1 expressing cells.

In this study, the cytotoxic agent Deruxtecan was conjugated to the exemplary antibodies of the present disclosure via a bridging anti-hFc antibody, which specifically binds to the Fc region of an antibody.

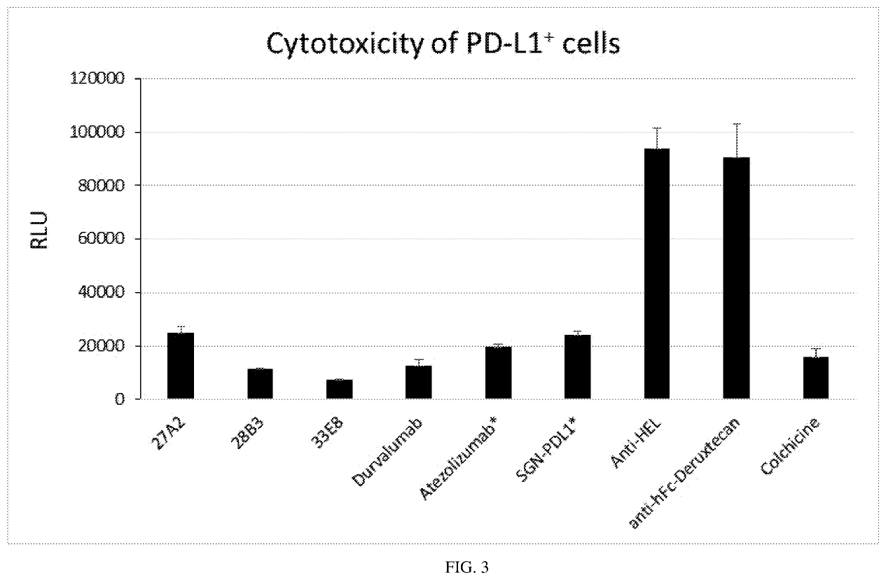

Briefly, PD-L1-expressing Raji cells (InvivoGen) were split into opaque-walled 96-well plate in 160 μl culture medium at 2500 cells/well density. Antibody conjugates were prepared by mixing 40 μl of 200 nM of anti-hFc antibody (Biolegend, Cat #410701) conjugated with Deruxtecan (MedKoo Bioscience, Cat #2071060) via stable thiol ether linkage (prepared by CellMosaic Inc.) with the followings:
(1) 500 nM of anti-PD-L1 antibodies of the present disclosure, the Fc region of which was human Fc (hFc), to be tested,
(2) 500 nM of commercially available anti-PD-L1 antibody Imfinzi (Durvalumab),
(3) 500 nM of analogues of anti-PD-L1 antibody Atezolizumab or SGN-PDL1 (analogue antibody data were marked with * in FIG. 3),
(4) 500 nM of anti-HEL isotype control antibody, or
(5) medium only.

The antibody conjugates were added in duplicate into the wells containing the Raji cells. The cells mixed with the antibody conjugates were incubated at 37° C., 5% CO2 for 3 days. Microtubule polymerization inhibitor Colchicine was used at 5 M as positive cytotoxicity control. After three days of incubation, one hundred microliter (100 μl) of freshly prepared CellTiter-Glo (Promega, Cat #G7571) was added to each well in the 96-well plate after removing 90 ul of supernatant from each well. Thereafter, the 96-well plate was placed on a orbital shaker for shaking at 120 rpm for 2 minutes to induce cell lysis. The cell lysates were incubated at room temperature for 10 minutes to stabilize luminescent signal before proceeding for measurement of luminescence signal on a plate reader (SpectraMax5) for cell viability. A direct relationship existed between the strength of the luminescence signal and the number of viable cells in each well. The results were shown in FIG. 3.

### Example 9: Epitope Binning of Anti-PD-L1 Antibodies

This study examines whether certain exemplary antibodies of the present invention belong to the same epitope bin of commercially available Avelumab, i.e., whether certain exemplary antibodies of the present disclosure bind to the same or similar epitope on the PD-L1 protein as Avelumab does.

Competition ELISA (cELISA) assay was used to determine whether the exemplary anti-PD-L1 antibodies of the present disclosure belong to the same epitope bin of Avelumab. Briefly, NUNC Maxisorp flat-bottom 96-well microplates were precoated with 0.5 μg/μl of recombinant human PD-L1-His protein (Sino Biologicals, Cat #10084-H08H) 1×PBS (GIBCO) and incubated overnight at 4° C. Plates were washed four times with 300 μl/well washing buffer (1×PBST, diluted from 20×PBST (Thermo Scientific) with distilled water)) and then blocked with 300 μl/well of blocking buffer (1×PBST/3% BSA) for 2 hours at room temperature on shaker (100 rpm). Plates were washed and added with 50 μl/well of diluted test anti-PD-L1 antibody starting at 20 μg/ml, followed by 11-point 1:2 serial dilution. Plates containing the anti-PD-L1 antibody solutions were incubated at 37° C. for one hour and then added 50 μl/well of 0.4 μg/ml. of Biotinylated Avelumab (Thermo Scientific™ EZ-Link™ Micro Sulfo-NHS-LC-Biotinylation Kit, Cat #0021935, prepared according to the manufacturer manual). Plates containing the mixtures were incubated at 37° C. for one hour. HRP-conjugated Avidin (Invitrogen, Cat #A2664) was used at 1:2000 dilution for detection. Avelumab was used as positive control.

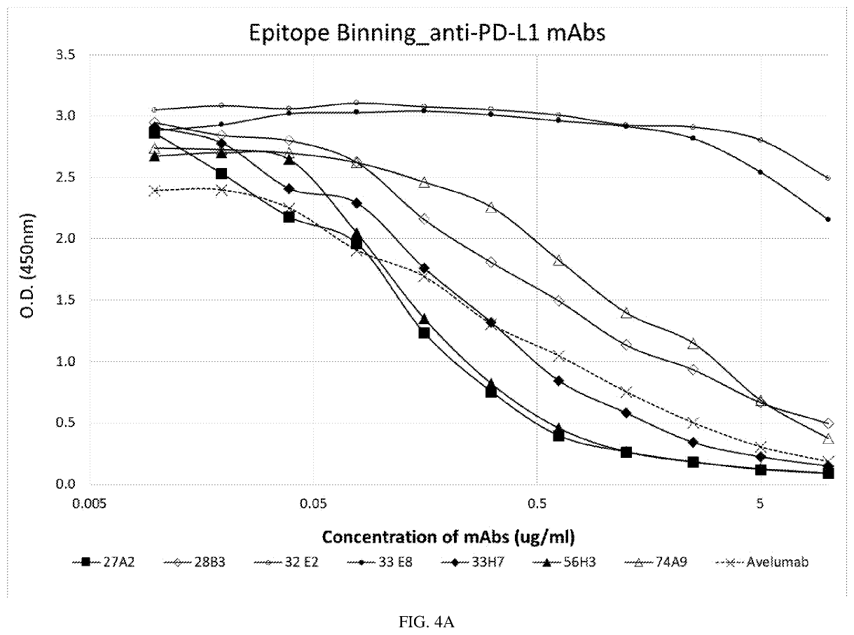

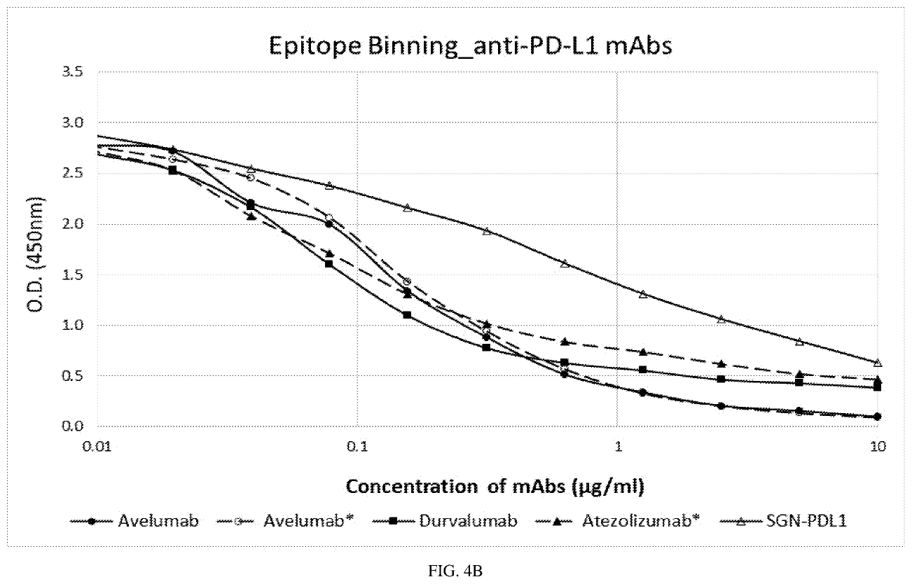

FIGS. 4A and 4B show the exemplary antibodies 27A2, 56H3, 33H7, 28H3, 74A9, Durvalumab, Avelumab and analogue Avelumab, Atezolizumab, SGN-PDL1 antibodies can effectively block the binding to human PD-L1-His protein by biotinylated Avelumab, indicating that these antibodies belong to same epitope bin. Antibodies 32E2 and 33E8 showed effective blocking of the binding of PD-L1 by Avelumab only at 5 μg/ml or higher concentration, indicating that these antibodies do not belong to the same epitope bin as Avelumab but their epitope bins may be related. Antibodies 55H3/76D6, 56E5, 57B10, 57F11, 62E6, 69D10, 73C8, 74F4 of the present disclosure do not show blocking of the binding to PD-L1 protein by Avelumab under the experiment condition (data not shown), indicating that these antibodies belong to distinct epitope bin as Avelumab.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

1. An isolated antibody, or antigen-binding fragment thereof, that binds to human programmed death ligand 1 (PD-L1), comprising
a heavy chain variable (V_{H}) domain comprising from N-terminus to C-terminus, three heavy chain complementarity-determining regions (CDRs), HCDR1, HCDR2, and HCDR3; and
a light chain variable (V_{L}) domain comprising from N-terminus to C-terminus, three light chain complementarity-determining regions (CDRs), LCDR1, LCDR2, and LCDR3; wherein (a) the HCDR1 comprises an amino acid sequence selected from the group consisting of X1-N-H-Y-M-X2 (SEQ ID NO:301), X3-X4-Y-Y-M-C (SEQ ID NO:303), N-N-Y-Y-M-S, (SEQ ID NO: 8), R-Y-F-Y-M-S (SEQ ID NO: 11), and S-A-Y-W-I-C (SEQ ID NO: 12); wherein X1 is N or S, X2 is C or S, X3 is A, N, or S, and X4 is A, N, or S;
(b) the HCDR2 comprises an amino acid sequence X32-I-X33-X34-G-S-X35-I-X36-D-Y-A-X37-W-A-K-G (SEQ ID NO: 226), wherein X32 is C or S, X33 is G or S, X34 is I, T, or V, X35 is A, D, G, or Y, X36 is S or T, and X37 is N or S;
(c) the HCDR3 comprises an amino acid sequence WTSGGGGFGL (SEQ ID NO: 42);
(d) the LCDR1 comprises an amino acid sequence selected from the group consisting of Q-S-S-Q-X65-X66-Y-X67-X68-Y-L-X69 (SEQ ID NO:239), Q-S-S-Q-X70-I-Y-S-D-Y-L-X71 (SEQ ID NO:240), and Q-S-S-Q-S-X72-Y-X73-N-Y-L-X74 (SEQ ID NO:241); wherein X65 is N, S, or T, X66 is I or V, X67 is N or S, X68 is D or N, and X69 is A, C, F, or S, X70 is N or T, X71 is F or S, X72 is I or V, X73 is N or S, X74 is A, C, or S;
(e) the LCDR2 comprises an amino acid sequence selected from the group consisting of X98-X99-X100-T-L-A-S (SEQ ID NO: 256), X101-X102-S-T-L-A-S (SEQ ID NO: 257), and S-T-A-T-L-A-S (SEQ ID NO: 72); wherein X98 is D, G, Q, S, or Y, X99 is A or T, X100 is A or S, X101 is D, G, Q, or Y, X102 is A or T; and
(f) the LCDR3 comprises an amino acid sequence Q-G-Y-Y-S-G-Y-I-W-T (SEQ ID NO: 83).

2-7. (canceled)

8. An isolated antibody, or antigen-binding fragment thereof, that binds to human programmed death ligand 1 (PD-L1), comprising
a heavy chain variable (V_{H}) domain comprising from N-terminus to C-terminus, three heavy chain complementarity-determining regions (CDRs), HCDR1, HCDR2, and HCDR3; and
a light chain variable (V_{L}) domain comprising from N-terminus to C-terminus, three light chain complementarity-determining regions (CDRs), LCDR1, LCDR2, and LCDR3; wherein
(a) the HCDR1 comprises an amino acid sequence X8-D-X9-Y-M-S (SEQ ID NO:306); wherein X8 is D or G, and X9 is W or Y;
(b) the HCDR2 comprises an amino acid sequence S-I-Y-X42-G-S-L-N-X43-Y-Y-A-T-W-A-K-G (SEQ ID NO: 229), wherein X42 is S or T, and X43 is I, S or T;
(c) the HCDR3 comprises an amino acid sequence R-X57-K-N-X58-D-X59-G-X60-F-D-L (SEQ ID NO: 237), wherein X57 is H, N, or T, X58 is A or G, X59 is W or Y, and X60 is H or Y;
(d) the LCDR1 comprises an amino acid sequence selected from the group consisting of Q-A-S-X76-S-I-X77-X78-X79-L-A (SEQ ID NO:243), Q-A-S-X80-S-I-N-D-R-L-A (SEQ ID NO: 244), Q-A-S-Q-S-I-X81-X82-A-L-A (SEQ ID NO:245), Q-A-S-Q-S-I-S-T-A-L-A (SEQ ID NO: 67), and Q-A-S-Q-S-I-G-N-A-L-A (SEQ ID NO: 68), wherein X76 is E or Q, X77 is G, N, or S, X78 is D, N, or T, X79 is A or R, X80 is E or Q, X81 is G or S, and X82 is N or T;
(e) the LCDR2 comprises an amino acid sequence X106-X107-S-T-L-A-S (SEQ ID NO: 260); wherein X106 is A or G, and X107 is A or T; and
(f) the LCDR3 comprises an amino acid sequence Q-Q-G-W-T-V-S-S-L-X111-N-A (SEQ ID NO:
263), wherein X111 is D or E.

9-14. (canceled)

15. An isolated antibody, or the antigen binding fragment thereof, that binds human PD-L1, comprising:
a heavy chain variable (V_{H}) domain comprising from N-terminus to C-terminus, three heavy chain complementarity-determining regions (CDRs), HCDR1, HCDR2, and HCDR3; and
a light chain variable (V_{L}) domain comprising from N-terminus to C-terminus, three light chain complementarity-determining regions (CDRs), LCDR1, LCDR2, and LCDR3; wherein
(a) the HCDR1 comprises an amino acid sequence selected from the group consisting of S-G-Y-D-M-S (SEQ ID NO: 15) and S-G-Y-D-M-C (SEQ ID NO: 16);
(b) the HCDR2 comprises an amino acid sequence X38-I-F-T-X39-S-G-S-T-W-Y-A-N-W-A-K-G (SEQ ID NO: 227), wherein X38 is C or S, X39 is G or T;
(c) the HCDR3 comprises an amino acid sequence T-X51-D-G-X52-G-S-F-Y-M-N-L (SEQ ID NO: 235); wherein X51 is K or R, and X52 is A or V;
(d) the LCDR1 comprises an amino acid sequence Q-A-S-G-T-I-G-S-N-L-A (SEQ ID NO: 63) or Q-A-S-Q-T-I-G-S-N-L-A (SEQ ID NO: 62);
(e) the LCDR2 comprises an amino acid sequence K-A-F-T-L-A-S (SEQ ID NO: 76) or K-T-F-T-L-A-S (SEQ ID NO: 77); and
(f) the LCDR3 comprises an amino acid sequence Q-Q-G-A-T-R-I-N-I-D-N-A (SEQ ID NO: 86 or Q-Q-G-A-S-R-I-N-I-D-N-A (SEQ ID NO: 87).

16. The isolated antibody, or the antigen binding fragment thereof, of claim 15, wherein
(a) the HCDR2 comprises an amino acid sequence as set forth in SEQ ID NO: 34 or 35; and
(b) the HCDR3 comprises an amino acid sequence as set forth in SEQ ID NO: 45 or 46.

17. The isolated antibody, or the antigen binding fragment thereof, of claim 16, wherein
(a) the HCDR1 comprises an amino acid sequence set forth in SEQ ID NO: 15, the HCDR2 comprising an amino acid sequence set forth in SEQ ID NO: 34, the HCDR3 comprising an amino acid sequence set forth in SEQ ID NO: 45, the LCDR1 comprising an amino acid sequence set forth in SEQ ID NO: 62, the LCDR2 comprising an amino acid sequence set forth in SEQ ID NO: 76, and the LCDR3 comprising an amino acid sequence set forth in SEQ ID NO: 86; or
(b) the HCDR1 comprises an amino acid sequence set forth in SEQ ID NO: 16, the HCDR2 comprising an amino acid sequence set forth in SEQ ID NO: 35, the HCDR3 comprising an amino acid sequence set forth in SEQ ID NO: 46, the LCDR1 comprising an amino acid sequence set forth in SEQ ID NO: 63, the LCDR2 comprising an amino acid sequence set forth in SEQ ID NO: 77, and the LCDR3 comprising an amino acid sequence set forth in SEQ ID NO: 87.

18. The isolated antibody, or the antigen binding fragment thereof, of claim 16, wherein the antibody comprises:
(a) a heavy chain variable region (HCVR) comprising an amino acid sequence set forth in SEQ ID NO: 106 or 107; and
(b) a light chain variable region (LCVR) comprising an amino acid set forth in SEQ ID NO: 132 or 133.

19. The isolated antibody, or the antigen binding fragment thereof, of claim 18, wherein the antibody comprises:
(a) the HCVR comprising an amino acid sequence set forth in SEQ ID NO: 106, and the LCVR comprising an amino acid sequence set forth in SEQ ID NO: 132; or
(b) the HCVR comprising an amino acid sequence set forth in SEQ ID NO: 107, and the LCVR comprising an amino acid sequence set forth in SEQ ID NO: 133.

20-21. (canceled)

22. An isolated antibody, or the antigen binding fragment thereof, that binds human PD-L1, comprising:
a heavy chain variable (V_{H}) domain comprising from N-terminus to C-terminus, three heavy chain complementarity-determining regions (CDRs), HCDR1, HCDR2, and HCDR3; and
a light chain variable (V_{L}) domain comprising from N-terminus to C-terminus, three light chain complementarity-determining regions (CDRs), LCDR1, LCDR2, and LCDR3; wherein
(a) the HCDR1 comprises an amino acid sequence selected from the group consisting of S-T-Y-A-L-G (SEQ ID NO: 17) and S-T-Y-A-M-G (SEQ ID NO: 18);
(b) the HCDR2 comprises an amino acid sequence S-I-S-I-G-G-A-T-Y-X40-A-X41-W-A-K-G (SEQ ID NO: 228), wherein X40 is F or Y, and X41 is S or T;
(c) the HCDR3 comprises an amino acid sequence A-R-N-V-D-X53-I-Y-L-X54-A-F-X55-X56 (SEQ ID NO: 236); wherein X53 is I or S, X54 is D or N, X55 is D or H, and X56 is I or T;
(d) the LCDR1 comprises an amino acid sequence Q-A-S-Q-N-I-Y-N-N-L-A (SEQ ID NO: 64);
(e) the LCDR2 comprises an amino acid sequence X104-X105-S-T-L-A-S (SEQ ID NO: 259), wherein X104 is R or S, and X105 is A or S; and
(f) the LCDR3 comprises an amino acid sequence Q-T-Y-Y-L-T-X109-T-X110-N-A (SEQ ID NO:262), wherein X109 is S or T, and X110 is I or T.

23-36. (canceled)

37. The isolated antibody, or the antigen binding fragment thereof, of claim 15, wherein the N-terminus of the heavy chain and/or light chain is a pyroglutamate (pE) residue.

38. The isolated antibody, or the antigen binding fragment thereof, of claim 15, wherein
(i) the antibody competes for binding to human PD-L1 with a monoclonal antibody of selected from the group consisting of 33E8, and 53G7;
(ii) the antibody specifically binds to a human and/or a cynomolgus PD-L1;
(iii) the antibody specifically binds to a cell surface human PD-L1;
(iv) the antibody blocks the interaction between PD-1 and PD-L1;
(v) the antibody improves an immune response;
(vi) the antibody induces ADCC; and
(vii) the antibody induces internalization of PD-L1.

39. (canceled)

40. The antibody, or the antigen binding fragment thereof, of any one of claim 15, wherein the antibody is a humanized antibody or a chimeric antibody.

41-42. (canceled)

43. An isolated polynucleotide encoding the antibody, or the antigen binding fragment thereof, of claim 15, an HCVR thereof, an LCVR thereof, a light chain thereof, a heavy chain thereof, or an antigen binding fragment thereof.

44-46. (canceled)

47. A pharmaceutical composition comprising the antibody, or the antigen binding fragment thereof, of claim 15, and a pharmaceutically acceptable carrier or diluent.

48. The pharmaceutical composition of claim 47, wherein the antibody, or the antigen binding fragment thereof, in the pharmaceutical composition is in an amount effective to (a) specifically bind to a cell surface human or cynomolgus PD-L1; (b) blocks the interaction between PD-1 and PD-L1; (c) induces ADCC; (d) induces internalization of PD-L1; e) improve the immune response of an immune cell; and (f) any combination of (a)-(e).

49-50. (canceled)

51. A method of inhibiting growth of a tumor in a subject, comprising administering an isolated antibody, or the antigen binding fragment thereof, of claim 15, thereby inhibiting growth of the tumor.

52. A method of treating cancer in a subject, comprising administering an isolated antibody, or the antigen binding fragment thereof, of claim 15, thereby treating the cancer.

53. (canceled)

54. The method of claim 52, further comprising administering an additional therapeutic agent.

55. The method of claim 54, wherein the additional therapeutic agent comprises an anti-tumor agent, radiotherapy, a chemotherapeutic agent, a surgery, a cancer vaccine, an agonist to a stimulatory receptor of an immune cell, a cytokine, a cell therapy, or a checkpoint inhibitor.

56-63. (canceled)

64. A method of killing a PD-L1 expressing cell, comprising contacting the cell with the isolated antibody, or the antigen binding fragment thereof, of claim 15, wherein the isolated antibody, or the antigen binding fragment thereof, is conjugated to a cytotoxic agent, thereby killing the PD-L1 expressing cell.

65-66. (canceled)

67. The method of claim 64, wherein the method is used to treat cancer in a subject.


SEQUENCE LISTING (313 sequences)

SEQ ID NO: 1 - protein, 6 aa, synthetic construct
  REGION 1..6: note = Synthetic
SNHYMS

SEQ ID NO: 2 - protein, 6 aa, synthetic construct
  REGION 1..6: note = Synthetic
SNHYMC

SEQ ID NO: 3 - protein, 6 aa, synthetic construct
  REGION 1..6: note = Synthetic
SAYYMC

SEQ ID NO: 4 - protein, 6 aa, synthetic construct
  REGION 1..6: note = Synthetic
ASYYMC

SEQ ID NO: 5 - protein, 6 aa, synthetic construct
  REGION 1..6: note = Synthetic
SNYYMC

SEQ ID NO: 6 - protein, 6 aa, synthetic construct
  REGION 1..6: note = Synthetic
NNHYMC

SEQ ID NO: 7 - protein, 6 aa, synthetic construct
  REGION 1..6: note = Synthetic
ANYYMC

SEQ ID NO: 8 - protein, 6 aa, synthetic construct
  REGION 1..6: note = Synthetic
NNYYMS

SEQ ID NO: 9 - protein, 6 aa, synthetic construct
  REGION 1..6: note = Synthetic
NNYYMC

SEQ ID NO: 10 - skipped

SEQ ID NO: 11 - protein, 6 aa, synthetic construct
  REGION 1..6: note = Synthetic
RYFYMS

SEQ ID NO: 12 - protein, 6 aa, synthetic construct
  REGION 1..6: note = Synthetic
SAYWIC

SEQ ID NO: 13 - protein, 6 aa, synthetic construct
  REGION 1..6: note = Synthetic
SSYYMS

SEQ ID NO: 14 - protein, 6 aa, synthetic construct
  REGION 1..6: note = Synthetic
SSYYMI

SEQ ID NO: 15 - protein, 6 aa, synthetic construct
  REGION 1..6: note = Synthetic
SGYDMS

SEQ ID NO: 16 - protein, 6 aa, synthetic construct
  REGION 1..6: note = Synthetic
SGYDMC

SEQ ID NO: 17 - protein, 6 aa, synthetic construct
  REGION 1..6: note = Synthetic
STYALG

SEQ ID NO: 18 - protein, 6 aa, synthetic construct
  REGION 1..6: note = Synthetic
STYAMG

SEQ ID NO: 19 - protein, 6 aa, synthetic construct
  REGION 1..6: note = Synthetic
GDYYMS

SEQ ID NO: 20 - protein, 6 aa, synthetic construct
  REGION 1..6: note = Synthetic
DDYYMS

SEQ ID NO: 21 - protein, 6 aa, synthetic construct
  REGION 1..6: note = Synthetic
GDWYMS

SEQ ID NO: 22 - protein, 17 aa, synthetic construct
  REGION 1..17: note = Synthetic
SIGVGSGITDYASWAKG

SEQ ID NO: 23 - protein, 17 aa, synthetic construct
  REGION 1..17: note = Synthetic
CIGVGSGITDYASWAKG

SEQ ID NO: 24 - protein, 17 aa, synthetic construct
  REGION 1..17: note = Synthetic
CIGIGSGITDYANWAEG

SEQ ID NO: 25 - protein, 17 aa, synthetic construct
  REGION 1..17: note = Synthetic
CIGIGSYITDYASWAKG

SEQ ID NO: 26 - protein, 17 aa, synthetic construct
  REGION 1..17: note = Synthetic
CIGIGSGISDYANWAKG

SEQ ID NO: 27 - protein, 17 aa, synthetic construct
  REGION 1..17: note = Synthetic
CIGIGSGITDYASWAKG

SEQ ID NO: 28 - protein, 17 aa, synthetic construct
  REGION 1..17: note = Synthetic
SIGIGSGITDYASWAKG

SEQ ID NO: 29 - protein, 17 aa, synthetic construct
  REGION 1..17: note = Synthetic
CIGIGSGISDYASWAKG

SEQ ID NO: 30 - protein, 17 aa, synthetic construct
  REGION 1..17: note = Synthetic
SISTGSDITDYASWAKG

SEQ ID NO: 31 - protein, 17 aa, synthetic construct
  REGION 1..17: note = Synthetic
CISTGSAITDYASWAKG

SEQ ID NO: 32 - protein, 18 aa, synthetic construct
  REGION 1..18: note = Synthetic
SISGGVTDNAYYASWAKG

SEQ ID NO: 33 - protein, 17 aa, synthetic construct
  REGION 1..17: note = Synthetic
YVYTGSGNTWYASWAKG

SEQ ID NO: 34 - protein, 17 aa, synthetic construct
  REGION 1..17: note = Synthetic
SIFTTSGSTWYANWAKG

SEQ ID NO: 35 - protein, 17 aa, synthetic construct
  REGION 1..17: note = Synthetic
CIFTGSGSTWYANWAKG

SEQ ID NO: 36 - protein, 16 aa, synthetic construct
  REGION 1..16: note = Synthetic
SISIGGATYYASWAKG

SEQ ID NO: 37 - protein, 16 aa, synthetic construct
  REGION 1..16: note = Synthetic
SISIGGATYFATWAKG

SEQ ID NO: 38 - protein, 17 aa, synthetic construct
  REGION 1..17: note = Synthetic
SIYTGSLNSYYATWAKG

SEQ ID NO: 39 - protein, 17 aa, synthetic construct
  REGION 1..17: note = Synthetic
SIYTGSLNTYYATWAKG

SEQ ID NO: 40 - protein, 17 aa, synthetic construct
  REGION 1..17: note = Synthetic
SIYTGSLNIYYATWAKG

SEQ ID NO: 41 - protein, 17 aa, synthetic construct
  REGION 1..17: note = Synthetic
SIYSGSLNTYYATWAKG

SEQ ID NO: 42 - protein, 10 aa, synthetic construct
  REGION 1..10: note = Synthetic
WTSGGGGFGL

SEQ ID NO: 43 - protein, 10 aa, synthetic construct
  REGION 1..10: note = Synthetic
DSSSGYFFLL

SEQ ID NO: 44 - protein, 14 aa, synthetic construct
  REGION 1..14: note = Synthetic
ASGADGVYDWGWDI

SEQ ID NO: 45 - protein, 12 aa, synthetic construct
  REGION 1..12: note = Synthetic
TKDGVGSFYMNL

SEQ ID NO: 46 - protein, 12 aa, synthetic construct
  REGION 1..12: note = Synthetic
TRDGAGSFYMNL

SEQ ID NO: 47 - protein, 14 aa, synthetic construct
  REGION 1..14: note = Synthetic
ARNVDSIYLDAFHT

SEQ ID NO: 48 - protein, 14 aa, synthetic construct
  REGION 1..14: note = Synthetic
ARNVDIIYLNAFDI

SEQ ID NO: 49 - protein, 12 aa, synthetic construct
  REGION 1..12: note = Synthetic
RNKNADYGHFDL

SEQ ID NO: 50 - protein, 12 aa, synthetic construct
  REGION 1..12: note = Synthetic
RHKNADYGHFDL

SEQ ID NO: 51 - protein, 12 aa, synthetic construct
  REGION 1..12: note = Synthetic
RNKNADWGHFDL

SEQ ID NO: 52 - protein, 12 aa, synthetic construct
  REGION 1..12: note = Synthetic
RTKNGDYGYFDL

SEQ ID NO: 53 - protein, 12 aa, synthetic construct
  REGION 1..12: note = Synthetic
QSSQTIYSDYLF

SEQ ID NO: 54 - protein, 12 aa, synthetic construct
  REGION 1..12: note = Synthetic
QSSQSVYSNYLS

SEQ ID NO: 55 - protein, 12 aa, synthetic construct
  REGION 1..12: note = Synthetic
QSSQSVYSNYLA

SEQ ID NO: 56 - protein, 12 aa, synthetic construct
  REGION 1..12: note = Synthetic
QSSQTIYSDYLS

SEQ ID NO: 57 - protein, 12 aa, synthetic construct
  REGION 1..12: note = Synthetic
QSSQNIYSDYLS

SEQ ID NO: 58 - protein, 12 aa, synthetic construct
  REGION 1..12: note = Synthetic
QSSQSIYNNYLS

SEQ ID NO: 59 - protein, 12 aa, synthetic construct
  REGION 1..12: note = Synthetic
QSSQSVYSNYLC

SEQ ID NO: 60 - protein, 11 aa, synthetic construct
  REGION 1..11: note = Synthetic
QASQNIYSNLA

SEQ ID NO: 61 - protein, 11 aa, synthetic construct
  REGION 1..11: note = Synthetic
QASQSIYSLLA

SEQ ID NO: 62 - protein, 11 aa, synthetic construct
  REGION 1..11: note = Synthetic
QASQTIGSNLA

SEQ ID NO: 63 - protein, 11 aa, synthetic construct
  REGION 1..11: note = Synthetic
QASGTIGSNLA

SEQ ID NO: 64 - protein, 11 aa, synthetic construct
  REGION 1..11: note = Synthetic
QASQNIYNNLA

SEQ ID NO: 65 - protein, 11 aa, synthetic construct
  REGION 1..11: note = Synthetic
QASESINDRLA

SEQ ID NO: 66 - protein, 11 aa, synthetic construct
  REGION 1..11: note = Synthetic
QASQSINDRLA

SEQ ID NO: 67 - protein, 11 aa, synthetic construct
  REGION 1..11: note = Synthetic
QASQSISTALA

SEQ ID NO: 68 - protein, 11 aa, synthetic construct
  REGION 1..11: note = Synthetic
QASQSIGNALA

SEQ ID NO: 69 - protein, 7 aa, synthetic construct
  REGION 1..7: note = Synthetic
QTSTLAS

SEQ ID NO: 70 - protein, 7 aa, synthetic construct
  REGION 1..7: note = Synthetic
YASTLAS

SEQ ID NO: 71 - protein, 7 aa, synthetic construct
  REGION 1..7: note = Synthetic
GASTLAS

SEQ ID NO: 72 - protein, 7 aa, synthetic construct
  REGION 1..7: note = Synthetic
STATLAS

SEQ ID NO: 73 - protein, 7 aa, synthetic construct
  REGION 1..7: note = Synthetic
DASTLAS

SEQ ID NO: 74 - protein, 7 aa, synthetic construct
  REGION 1..7: note = Synthetic
GASNLRS

SEQ ID NO: 75 - protein, 7 aa, synthetic construct
  REGION 1..7: note = Synthetic
GASNLES

SEQ ID NO: 76 - protein, 7 aa, synthetic construct
  REGION 1..7: note = Synthetic
KAFTLAS

SEQ ID NO: 77 - protein, 7 aa, synthetic construct
  REGION 1..7: note = Synthetic
KTFTLAS

SEQ ID NO: 78 - protein, 7 aa, synthetic construct
  REGION 1..7: note = Synthetic
RASTLAS

SEQ ID NO: 79 - protein, 7 aa, synthetic construct
  REGION 1..7: note = Synthetic
SSSTLAS

SEQ ID NO: 80 - protein, 7 aa, synthetic construct
  REGION 1..7: note = Synthetic
AASTLAS

SEQ ID NO: 81 - skipped

SEQ ID NO: 82 - protein, 7 aa, synthetic construct
  REGION 1..7: note = Synthetic
ATSTLAS

SEQ ID NO: 83 - protein, 10 aa, synthetic construct
  REGION 1..10: note = Synthetic
QGYYSGYIWT

SEQ ID NO: 84 - protein, 12 aa, synthetic construct
  REGION 1..12: note = Synthetic
QEGYSIGNVDNP

SEQ ID NO: 85 - protein, 13 aa, synthetic construct
  REGION 1..13: note = Synthetic
QNNYDSGRIYGLA

SEQ ID NO: 86 - protein, 12 aa, synthetic construct
  REGION 1..12: note = Synthetic
QQGATRINIDNA

SEQ ID NO: 87 - protein, 12 aa, synthetic construct
  REGION 1..12: note = Synthetic
QQGASRINIDNA

SEQ ID NO: 88 - protein, 11 aa, synthetic construct
  REGION 1..11: note = Synthetic
QTYYLTTTTNA

SEQ ID NO: 89 - protein, 11 aa, synthetic construct
  REGION 1..11: note = Synthetic
QTYYLTSTINA

SEQ ID NO: 90 - protein, 12 aa, synthetic construct
  REGION 1..12: note = Synthetic
QQGWTVSSLDNA

SEQ ID NO: 91 - protein, 12 aa, synthetic construct
  REGION 1..12: note = Synthetic
QQGWTVSSLENA

SEQ ID NO: 92 - protein, 119 aa, synthetic construct
  REGION 1..119: note = Synthetic
QEQLEESGGGLVQPEGSLTLTCTASGFSFSSNHYMSWVRQAPGKGLEWIGSIGVGSGITDYASWAKGRFTISKASSTTLTLQMTTLTAADTAIYFCARWTSGGGGFGLWGPGTLVTVSS

SEQ ID NO: 93 - protein, 119 aa, synthetic construct
  REGION 1..119: note = Synthetic
QEQLEESGGGLVQPEGSLTLTCTASGFSFSSNHYMCWVRQAPGKGLEWIGCIGVGSGITDYASWAKGRFTISKASSTTLTLQMTTLTAADTAIYFCARWTSGGGGFGLWGPGTLVTVSS

SEQ ID NO: 94 - protein, 119 aa, synthetic construct
  REGION 1..119: note = Synthetic
QEQLVESGGGLVQPEGSLTLTCTASGFSFSSAYYMCWVRQAPGKGLEWVGCIGVGSGITDYASWAKGRFTISKTSSTTVTLQMTTLTAADTAIYFCARWTSGGGGFGLWGPGTLVTVSS

SEQ ID NO: 95 - protein, 118 aa, synthetic construct
  REGION 1..118: note = Synthetic
QEQLVESGGGLVQPEGSLTLTCTASGFSFSASYYMCWVRQAPGKGLEWIGCIGIGSGITDYANWAEGRFTISKTSTTVTLQMTTLTAADTAIYFCARWTSGGGGFGLWGPGTLVTVSS

SEQ ID NO: 96 - protein, 119 aa, synthetic construct
  REGION 1..119: note = Synthetic
QEQLVESGGGLVQPEGSLTLTCTASEFSFSSNYYMCWVRQAPGKGLEWIGCIGIGSYITDYASWAKGRFTISKTSSTTVTLQMTTLTAADTAIYFCARWTSGGGGFGLWGPGTLVTVSS

SEQ ID NO: 97 - protein, 119 aa, synthetic construct
  REGION 1..119: note = Synthetic
QEQLVESGGGLVQPEGSLTLTCTASAFSFNNNHYMCWVRQAPGKGLEWIGCIGIGSGISDYANWAKGRFTISKTSSTTVTLQMTTLTAADTAIYFCARWTSGGGGFGLWGPGTLVTVSS

SEQ ID NO: 98 - protein, 119 aa, synthetic construct
  REGION 1..119: note = Synthetic
QEQLVESGGGLVQPEGSLTLTCTASEFSFGANYYMCWVRQAPGKGLEWIGCIGIGSGITDYASWAKGRFTISKTSSTTVTLQMTTLTAADTAIYFCARWTSGGGGFGLWGPGTLVTVSS

SEQ ID NO: 99 - protein, 119 aa, synthetic construct
  REGION 1..119: note = Synthetic
QEQLVESGGGLVQPEGSLTLTCTASEFSFSNNYYMSWVRQAPGKGLEWLGSIGIGSGITDYASWAKGRFTISKASSTTVTLQMTTLTAADTAIYFCARWTSGGGGFGLWGPGTLVTVSS

SEQ ID NO: 100 - protein, 119 aa, synthetic construct
  REGION 1..119: note = Synthetic
QEQLVESGGGLVQPEGSLTLTCTASEFSFSNNYYMCWVRQAPGKGLEWLGCIGIGSGITDYASWAKGRFTISKASSTTVTLQMTTLTAADTAIYFCARWTSGGGGFGLWGPGTLVTVSS

SEQ ID NO: 101 - protein, 119 aa, synthetic construct
  REGION 1..119: note = Synthetic
QEQLVESGGGLVQPEGSLTLTCTASEFSFSSNYYMCWVRQAPGKGLEWIGCIGIGSGISDYASWAKGRFTISKASSTTVTLQMTTLTAADTAIYFCARWTSGGGGFGLWGPGTLVTVSS

SEQ ID NO: 102 - protein, 119 aa, synthetic construct
  REGION 1..119: note = Synthetic
QEQLVESGGGLVQPEGSLTVTCTASGFDLSRYFYMSWVRQAPGKGLEWIASISTGSDITDYASWAKGRFTISKTSSTTVTLQMTTLTAADTAIYFCARWTSGGGGFGLWGPGTLVTVSS

SEQ ID NO: 103 - protein, 119 aa, synthetic construct
  REGION 1..119: note = Synthetic
QEQLEESGGGLVQPEGSLTLTCTASGFDFSSAYWICWVRQAPGKGLEWIGCISTGSAITDYASWAKGRFTISKTSSTTVTLQMTTLTAADTAIYFCARWTSGGGGFGLWGPGTLVTVSS

SEQ ID NO: 104 - protein, 120 aa, synthetic construct
  REGION 1..120: note = Synthetic
QEQLEESGGDLVKPEGSLTLTCTASGFSFSSSYYMSWVRQAPGMGLEWIASISGGVTDNAYYASWAKGRFTVSKTSSTTVTLQLTSLTAADTATYFCARDSSSGYFFLLWGPGTLVTVSS

SEQ ID NO: 105 - protein, 118 aa, synthetic construct
  REGION 1..118: note = Synthetic
QSVEESGGRLVTPGTPLTLSCTASGFSLSSYYMIWVRQAPGKGLEYIGYVYTGSGNTWYASWAKGRFAISKASTTVDLMITSPTTEDTATYFCASGADGVYDWGWDIWGPGTLVTVSL

SEQ ID NO: 106 - protein, 118 aa, synthetic construct
  REGION 1..118: note = Synthetic
QSLEESGGDLVKPGASLTLTCTASGFYFSSGYDMSWVRQAPGKGLEWIASIFTTSGSTWYANWAKGRFTISKTSSTTVTLQMTSLTDADTATYFCTKDGVGSFYMNLWGPGTLVTVSS

SEQ ID NO: 107 - protein, 118 aa, synthetic construct
  REGION 1..118: note = Synthetic
QSLEESGGDLVKPGASLTLTCTASGFYFSSGYDMCWVRQAPGKGLEWIACIFTGSGSTWYANWAKGRFTISKTSSTTVTLQMTSLTDADTATYFCTRDGAGSFYMNLWGPGTLVTVSS

SEQ ID NO: 108 - protein, 117 aa, synthetic construct
  REGION 1..117: note = Synthetic
QSVEESGGRLVTPGTPLTLTCAVSGFSLSTYALGWVRQAPGKGLEWIGSISIGGATYYASWAKGRFTISKTSTTVDLKIASPTTEDTATYFCARNVDSIYLDAFHTWGPGTLVTVSL

SEQ ID NO: 109 - protein, 117 aa, synthetic construct
  REGION 1..117: note = Synthetic
QSVEESGGRLVTPGTPLTLTCTVSGIDLSTYAMGWVRQAPGKGLEWIGSISIGGATYFATWAKGRFTISKGSTTVDLKIASPTTEDTATYFCARNVDIIYLNAFDIWGPGTLVTVSL

SEQ ID NO: 110 - protein, 121 aa, synthetic construct
  REGION 1..121: note = Synthetic
QEQLEESGGGLVQPEGSLTLTCTVSGFSFSGDYYMSWVRQAPGKGLEWVASIYTGSLNSYYATWAKGRFTISLTSSTTVTLQMTSLTAADTATYFCARRNKNADYGHFDLWGPGTLVTVSS

SEQ ID NO: 111 - protein, 121 aa, synthetic construct
  REGION 1..121: note = Synthetic
QEQLEESGGGLVQPEGSLTLTCTASGFSFSGDYYMSWVRQAPGKGLEWIASIYTGSLNTYYATWAKGRFTISLTSSTTVTLQMTSLTAADTATYFCARRHKNADYGHFDLWGPGTLVTVSS

SEQ ID NO: 112 - protein, 121 aa, synthetic construct
  REGION 1..121: note = Synthetic
QEQLEESGGGLVQPEGSLTLTCTASGFSFSDDYYMSWVRQAPGKGLEWIASIYTGSLNTYYATWAKGRFTISLTSSTTVTLQMTSLTAADTATYFCARRNKNADWGHFDLWGPGTLVTVSS

SEQ ID NO: 113 - protein, 121 aa, synthetic construct
  REGION 1..121: note = Synthetic
QEHLEESGGGLVQPEGALTLTCTASGFSFSGDYYMSWVRQAPGKGLEWIASIYTGSLNTYYATWAKGRFTISIASSTTVTLQVTSLTAADTATYFCARRTKNGDYGYFDLWGPGTLVTVSS

SEQ ID NO: 114 - protein, 121 aa, synthetic construct
  REGION 1..121: note = Synthetic
QEQLEESGGDLVKPEGSLTLTCTASGFSFSGDYYMSWVRQAPGKGLEWIASIYTGSLNIYYATWAKGRFTISLTSSTTVTLQMTSLTAADTATYFCARRNKNADYGHFDLWGPGTLVTVSS

SEQ ID NO: 115 - protein, 121 aa, synthetic construct
  REGION 1..121: note = Synthetic
QEQLEESGGGLVQPEGSLTLTCTASGFSFSGDWYMSWVRQAPGKGLEWIASIYSGSLNTYYATWAKGRFTVSLTSSTTVTLQMTSLTAADTATYFCARRNKNADYGHFDLWGPGTLVTVSS

SEQ ID NO: 116 - protein, 121 aa, synthetic construct
  REGION 1..121: note = Synthetic
QEQLEESGGGLVQPEGSLTLTCTVSGFSFSGDYYMSWVRQAPGKGLEWVASIYTGSLNTYYATWAKGRFTISLTSSTTVTLQMTSLTAADTATYFCARRNKNADYGHFDLWGPGTLVTVSS

SEQ ID NO: 117 - protein, 121 aa, synthetic construct
  REGION 1..121: note = Synthetic
QEQLEESGGGLVQPEGSLTLTCTASGFSFSGDYYMSWVRQAPGKGLEWIASIYTGSLNTYYATWAKGRFTISLTSSTTVTLQMTSLTAADTATYFCARRNKNADYGHFDLWGPGTLVTVSS

SEQ ID NO: 118 - protein, 108 aa, synthetic construct
  REGION 1..108: note = Synthetic
QVLTQTSSPVSAAVGGTVTINCQSSQTIYSDYLFWYQQTPGQPPKLLIYQTSTLASGVPSRFKGSGSGTEFTLTISEIQCDDAATYYCQGYYSGYIWTFGGGTEVVVR

SEQ ID NO: 119 - protein, 108 aa, synthetic construct; identical to SEQ ID NO: 118
  REGION 1..108: note = Synthetic

SEQ ID NO: 120 - protein, 108 aa, synthetic construct
  REGION 1..108: note = Synthetic
QVLTQTASPVSAAVGGTVTISCQSSQSVYSNYLSWYQQKPGQPPKLLIYYASTLASGVPSRFKGSGSGTQFTLTISGVQCDDAATYYCQGYYSGYIWTFGGGTEVVVK

SEQ ID NO: 121 - protein, 108 aa, synthetic construct
  REGION 1..108: note = Synthetic
QVLTQTASPVSAAVGSTVTINCQSSQSVYSNYLAWYQQKPGQPPNLLIYGASTLASGVPSRFKGSGSGTQFTLTISDLQCDDAATYYCQGYYSGYIWTFGGGTEVVVK

SEQ ID NO: 122 - protein, 108 aa, synthetic construct
  REGION 1..108: note = Synthetic
QVLTQTSSPVSAAVGGTVTINCQSSQTIYSDYLSWYQQKPGQPPKLLIYQTSTLASGVPSRFKGSGSGTEFTLTIREIQCDDAATYYCQGYYSGYIWTFGGGTEVVVK

SEQ ID NO: 123 - protein, 108 aa, synthetic construct
  REGION 1..108: note = Synthetic
QVLTQTASPVSAAVGSTVTINCQSSQNIYSDYLSWYQQKPGQPPKLLIYGASTLASGVPSRFKGNGSGTQFTLTISDLQCDDAATYYCQGYYSGYIWTFGGGTEVVVK

SEQ ID NO: 124 - protein, 108 aa, synthetic construct
  REGION 1..108: note = Synthetic
AVLTQTPSPVSAAVGGTVTINCQSSQTIYSDYLSWYQQKPGQPPKLLIYQTSTLASGVPSRFKGSGSGTEFTLTISEIQCDDAATYYCQGYYSGYIWTFGGGTEVVVK

SEQ ID NO: 125 - protein, 108 aa, synthetic construct
  REGION 1..108: note = Synthetic
QVLTQTSSPVSAAVGGTVTINCQSSQTIYSDYLFWYQQKPGQPPKLLIYQTSTLASGVPSRFSGSGSGTQFTLTISGVQCDDAATYYCQGYYSGYIWTFGGGTEVVVN

SEQ ID NO: 126 - protein, 108 aa, synthetic construct; identical to SEQ ID NO: 125
  REGION 1..108: note = Synthetic

SEQ ID NO: 127 - protein, 108 aa, synthetic construct
  REGION 1..108: note = Synthetic
QVLTQTSSPVSAAVGGTVTISCQSSQTIYSDYLFWYQQKPGQPPKLLIYQTSTLASGVPSRFKGSGSGTEFTLTISEMQCDDAATYYCQGYYSGYIWTFGGGTEVVVK

SEQ ID NO: 128 - protein, 108 aa, synthetic construct
  REGION 1..108: note = Synthetic
QVLTQTASPVSAAVGGTVTLNCQSSQSIYNNYLSWYQQRAGQPPKLLIYSTATLASGVPSRFKGSGSGTQFTLTISEIQCEDAATYYCQGYYSGYIWTFGGGTEVVVK

SEQ ID NO: 129 - protein, 108 aa, synthetic construct
  REGION 1..108: note = Synthetic
QVLTQTASPVSAAVGGTVTINCQSSQSVYSNYLCWYQQKPGQPPKLLMYDASTLASGVPSRFKGSGSGTQFTLTISGVQCDDAATYYCQGYYSGYIWTFGGGTEVVVK

SEQ ID NO: 130 - protein, 109 aa, synthetic construct
  REGION 1..109: note = Synthetic
LVMTQTPSSVSAAVGGTVTINCQASQNIYSNLAWYQQKPGQRPKLLIFGASNLRSGVPSRFKGSGSGTEYTLTISDLECADAATYYCQEGYSIGNVDNPFGGGTEVVVK

SEQ ID NO: 131 - protein, 111 aa, synthetic construct
  REGION 1..111: note = Synthetic
DIVMTQTPASVEAAVGDTVTIKCQASQSIYSLLAWYQQKPGQRPKLLIAGASNLESGVPSRFSGSGSGTEYTLTISALECADAATYYCQNNYDSGRIYGLAFGGGTEVVVK

SEQ ID NO: 132 - protein, 109 aa, synthetic construct
  REGION 1..109: note = Synthetic
LVMTQTPASVEVAVGGTVTIKCQASQTIGSNLAWYQQRPGQPPKLLIYKAFTLASGVSSRFKGSGSGTQFTLTISGVECADAATYYCQQGATRINIDNAFGGGTEVVVK

SEQ ID NO: 133 - protein, 109 aa, synthetic construct
  REGION 1..109: note = Synthetic
LVMTQTPASVEVPVGGTVTIKCQASGTIGSNLAWYQQKPGQPPKLLIYKTFTLASGVSSRFKGSGSGTEFTLTISGVECADAATYYCQQGASRINIDNAFGGGTEVVVK

SEQ ID NO: 134 - protein, 109 aa, synthetic construct
  REGION 1..109: note = Synthetic
DIVMTQTPASVEAAVGGTVTIKCQASQNIYNNLAWYQQKPGQPPKLLIYRASTLASGVPSRFKGSGSGTEYTLTISDLECDDAATYYCQTYYLTTTTNAFGGGTEVVVK

SEQ ID NO: 135 - protein, 109 aa, synthetic construct
  REGION 1..109: note = Synthetic
DIVMTQTPASVEAAVGGTVTIKCQASQNIYNNLAWYQQKPGQPPKLLIYSSSTLASGVSSRFKGSGSGTEYTLTISDLECADAATYYCQTYYLTSTINAFGGGTEVVVK

SEQ ID NO: 136 - protein, 109 aa, synthetic construct
  REGION 1..109: note = Synthetic
YDMTQTPASVEVAVGGTVTIKCQASESINDRLAWYQQKPGQPPKLLIYAASTLASGVPSRFRGSGSGTDFTLTISDLECADAATYYCQQGWTVSSLDNAFGGGTEVVAM

SEQ ID NO: 137 - protein, 109 aa, synthetic construct
  REGION 1..109: note = Synthetic
YDMTQTPGSVEVAVGGTVTIKCQASQSINDRLAWYQQTPGQPPKLLIYGASTLASGVPSRFKGSGSGTEFTLTISDLECADAATYYCQQGWTVSSLDNAFGGGTEVVAK

SEQ ID NO: 138 - protein, 109 aa, synthetic construct
  REGION 1..109: note = Synthetic
YDMTQTPASVEVAVGGTVTIKCQASQSISTALAWYQQKPGQPPKLLIYAASTLASGVPSRFRGSGSGTDFTLTISDLECADAATYYCQQGWTVSSLDNAFGGGTEVVAM

SEQ ID NO: 139 - protein, 109 aa, synthetic construct
  REGION 1..109: note = Synthetic
YDMTQTPASVEVAVGGTVTIKCQASQSINDRLAWYQQKPGQPPKLLIYAASTLASGVPSRFKGSGSGTEFTLTISDLECADAATYYCQQGWTVSSLENAFGGGTEVVAK

SEQ ID NO: 140 - protein, 109 aa, synthetic construct
  REGION 1..109: note = Synthetic
YDMTQTPASVEVAVGGTVTIKCQASQSINDRLAWYQQKPRQPPKLLIYGASTLASGVPSRFKGSGSGTEFTLTISDLECVDAATYYCQQGWTVSSLDNAFGGGTEVVAK

SEQ ID NO: 141 - protein, 109 aa, synthetic construct
  REGION 1..109: note = Synthetic
YDMTQTPASVEVAVGGTVTIKCQASQSIGNALAWYQQKPGQPPKLLIYATSTLASGVPSRFKGSGSGTEFTLTISDLECGDAATYYCQQGWTVSSLDNAFGGGTEVVAK

SEQ ID NO: 142 - protein, 109 aa, synthetic construct
  REGION 1..109: note = Synthetic
YDMTQTPASVEVAVGGTVTIKCQASQSINDRLAWYQQKPGQPPKLLIYAASTLASGVPSRFRGSGSGTDFTLTISDLECADAATYYCQQGWTVSSLDNAFGGGTEVVAM

SEQ ID NO: 143 - protein, 109 aa, synthetic construct
  REGION 1..109: note = Synthetic
YDMTQTPASVEVAVGGTVTIKCQASQSINDRLAWYQQSPGQPPKLLIYGASTLASGVPSRFKGSGSGTDFTLTISDLECADAATYYCQQGWTVSSLDNAFGGGTEVVAK

SEQ ID NO: 144 - protein, 9 aa, synthetic construct
  REGION 1..9: note = Synthetic
GFSFSSNHY

SEQ ID NO: 145 - protein, 9 aa, synthetic construct
  REGION 1..9: note = Synthetic
GFSFSSAYY

SEQ ID NO: 146 - protein, 9 aa, synthetic construct
  REGION 1..9: note = Synthetic
GFSFSASYY

SEQ ID NO: 147 - protein, 9 aa, synthetic construct
  REGION 1..9: note = Synthetic
EFSFSSNYY

SEQ ID NO: 148 - protein, 9 aa, synthetic construct
  REGION 1..9: note = Synthetic
AFSFNNNHY

SEQ ID NO: 149 - protein, 9 aa, synthetic construct
  REGION 1..9: note = Synthetic
EFSFGANYY

SEQ ID NO: 150 - protein, 9 aa, synthetic construct
  REGION 1..9: note = Synthetic
EFSFSNNYY

SEQ ID NO: 151 - protein, 9 aa, synthetic construct
  REGION 1..9: note = Synthetic
GFDLSRYFY

SEQ ID NO: 152 - protein, 9 aa, synthetic construct
  REGION 1..9: note = Synthetic
GFDFSSAYW

SEQ ID NO: 153 - protein, 9 aa, synthetic construct
  REGION 1..9: note = Synthetic
GFSFSSSYY

SEQ ID NO: 154 - protein, 8 aa, synthetic construct
  REGION 1..8: note = Synthetic
GFSLSSYY

SEQ ID NO: 155 - protein, 9 aa, synthetic construct
  REGION 1..9: note = Synthetic
GFYFSSGYD

SEQ ID NO: 156 - protein, 8 aa, synthetic construct
  REGION 1..8: note = Synthetic
GFSLSTYA

SEQ ID NO: 157 - protein, 8 aa, synthetic construct
  REGION 1..8: note = Synthetic
GIDLSTYA

SEQ ID NO: 158 - protein, 9 aa, synthetic construct
  REGION 1..9: note = Synthetic
GFSFSGDYY

SEQ ID NO: 159 - protein, 9 aa, synthetic construct
  REGION 1..9: note = Synthetic
GFSFSDDYY

SEQ ID NO: 160 - protein, 9 aa, synthetic construct
  REGION 1..9: note = Synthetic
GFSFSGDWY

SEQ ID NO: 161 - protein, 7 aa, synthetic construct
  REGION 1..7: note = Synthetic
IGVGSGI

SEQ ID NO: 162 - protein, 7 aa, synthetic construct
  REGION 1..7: note = Synthetic
IGIGSGI

SEQ ID NO: 163 - protein, 7 aa, synthetic construct
  REGION 1..7: note = Synthetic
ISTGSDI

SEQ ID NO: 164 - protein, 7 aa, synthetic construct
  REGION 1..7: note = Synthetic
ISTGSAI

SEQ ID NO: 165 - protein, 7 aa, synthetic construct
  REGION 1..7: note = Synthetic
ISGGVTD

SEQ ID NO: 166 - protein, 7 aa, synthetic construct
  REGION 1..7: note = Synthetic
VYTGSGN

SEQ ID NO: 167 - protein, 7 aa, synthetic construct
  REGION 1..7: note = Synthetic
IFTTSGS

SEQ ID NO: 168 - protein, 7 aa, synthetic construct
  REGION 1..7: note = Synthetic
IFTGSGS

SEQ ID NO: 169 - protein, 7 aa, synthetic construct
  REGION 1..7: note = Synthetic
ISIGGAT

SEQ ID NO: 170 - protein, 7 aa, synthetic construct
  REGION 1..7: note = Synthetic
IYTGSLN

SEQ ID NO: 171 - protein, 7 aa, synthetic construct
  REGION 1..7: note = Synthetic
IYSGSLN

SEQ ID NO: 172 - protein, 12 aa, synthetic construct
  REGION 1..12: note = Synthetic
ARWTSGGGGFGL

SEQ ID NO: 173 - protein, 12 aa, synthetic construct
  REGION 1..12: note = Synthetic
ARDSSSGYFFLL

SEQ ID NO: 174 - protein, 14 aa, synthetic construct
  REGION 1..14: note = Synthetic
ARRNKNADYGHFDL

SEQ ID NO: 175 - protein, 14 aa, synthetic construct
  REGION 1..14: note = Synthetic
ARRHKNADYGHFDL

SEQ ID NO: 176 - protein, 14 aa, synthetic construct
  REGION 1..14: note = Synthetic
ARRNKNADWGHFDL

SEQ ID NO: 177 - protein, 14 aa, synthetic construct
  REGION 1..14: note = Synthetic
ARRTKNGDYGYFDL

SEQ ID NO: 178 - protein, 11 aa, synthetic construct
  REGION 1..11: note = Synthetic
GFSFSSNHYMS

SEQ ID NO: 179 - protein, 11 aa, synthetic construct
  REGION 1..11: note = Synthetic
GFSFSSNHYMC

SEQ ID NO: 180 - protein, 11 aa, synthetic construct
  REGION 1..11: note = Synthetic
GFSFSSAYYMC

SEQ ID NO: 181 - protein, 11 aa, synthetic construct
  REGION 1..11: note = Synthetic
GFSFSASYYMC

SEQ ID NO: 182 - protein, 11 aa, synthetic construct
  REGION 1..11: note = Synthetic
EFSFSSNYYMC

SEQ ID NO: 183 - protein, 11 aa, synthetic construct
  REGION 1..11: note = Synthetic
AFSFNNNHYMC

SEQ ID NO: 184 - protein, 11 aa, synthetic construct
  REGION 1..11: note = Synthetic
EFSFGANYYMC

SEQ ID NO: 185 - protein, 11 aa, synthetic construct
  REGION 1..11: note = Synthetic
EFSFSNNYYMS

SEQ ID NO: 186 - protein, 11 aa, synthetic construct
  REGION 1..11: note = Synthetic
EFSFSNNYYMC

SEQ ID NO: 187 - protein, 11 aa, synthetic construct
  REGION 1..11: note = Synthetic
GFDLSRYFYMS

SEQ ID NO: 188 - protein, 11 aa, synthetic construct
  REGION 1..11: note = Synthetic
GFDFSSAYWIC

SEQ ID NO: 189 - protein, 11 aa, synthetic construct
  REGION 1..11: note = Synthetic
GFSFSSSYYMS

SEQ ID NO: 190 - protein, 10 aa, synthetic construct
  REGION 1..10: note = Synthetic
GFSLSSYYMI

SEQ ID NO: 191 - protein, 11 aa, synthetic construct
  REGION 1..11: note = Synthetic
GFYFSSGYDMS

SEQ ID NO: 192 - protein, 11 aa, synthetic construct
  REGION 1..11: note = Synthetic
GFYFSSGYDMC

SEQ ID NO: 193 - protein, 10 aa, synthetic construct
  REGION 1..10: note = Synthetic
GFSLSTYALG

SEQ ID NO: 194 - protein, 10 aa, synthetic construct
  REGION 1..10: note = Synthetic
GIDLSTYAMG

SEQ ID NO: 195 - protein, 11 aa, synthetic construct
  REGION 1..11: note = Synthetic
GFSFSGDYYMS

SEQ ID NO: 196 - protein, 11 aa, synthetic construct
  REGION 1..11: note = Synthetic
GFSFSDDYYMS

SEQ ID NO: 197 - protein, 11 aa, synthetic construct
  REGION 1..11: note = Synthetic
GFSFSGDWYMS

SEQ ID NO: 198 - protein, 7 aa, synthetic construct
  REGION 1..7: note = Synthetic
QTIYSDY

SEQ ID NO: 199 - protein, 7 aa, synthetic construct
  REGION 1..7: note = Synthetic
QSVYSNY

SEQ ID NO: 200 - protein, 7 aa, synthetic construct
  REGION 1..7: note = Synthetic
QNIYSDY

SEQ ID NO: 201 - protein, 7 aa, synthetic construct
  REGION 1..7: note = Synthetic
QSIYNNY

SEQ ID NO: 202 - protein, 6 aa, synthetic construct
  REGION 1..6: note = Synthetic
QNIYSN

SEQ ID NO: 203 - protein, 6 aa, synthetic construct
  REGION 1..6: note = Synthetic
QSIYSL

SEQ ID NO: 204 - protein, 6 aa, synthetic construct
  REGION 1..6: note = Synthetic
QTIGSN

SEQ ID NO: 205 - protein, 6 aa, synthetic construct
  REGION 1..6: note = Synthetic
GTIGSN

SEQ ID NO: 206 - protein, 6 aa, synthetic construct
  REGION 1..6: note = Synthetic
QNIYNN

SEQ ID NO: 207 - protein, 6 aa, synthetic construct
  REGION 1..6: note = Synthetic
ESINDR

SEQ ID NO: 208 - protein, 6 aa, synthetic construct
  REGION 1..6: note = Synthetic
QSINDR

SEQ ID NO: 209 - protein, 6 aa, synthetic construct
  REGION 1..6: note = Synthetic
QSISTA

SEQ ID NO: 210 - protein, 6 aa, synthetic construct
  REGION 1..6: note = Synthetic
QSIGNA

SEQ ID NO: 211-221 - skipped (11 entries)

SEQ ID NO: 222 - protein, 9 aa, synthetic construct
  REGION 1..9: note = Synthetic
  VARIANT 6: note = X is A or S
  VARIANT 7: note = X is A, N, or S
  VARIANT 8: note = X is H or Y
GFSFSXXXY

SEQ ID NO: 223 - protein, 9 aa, synthetic construct
  REGION 1..9: note = Synthetic
  VARIANT 5: note = X is G or S
  VARIANT 6: note = X is A, N, or S
EFSFXXNYY

SEQ ID NO: 224 - protein, 8 aa, synthetic construct
  REGION 1..8: note = Synthetic
  VARIANT 2: note = X is F or I
  VARIANT 3: note = X is D or S
GXXLSTYA

SEQ ID NO: 225 - protein, 9 aa, synthetic construct
  REGION 1..9: note = Synthetic
  VARIANT 6: note = X is D or G
  VARIANT 8: note = X is W or Y
GFSFSXDXY

SEQ ID NO: 226 - protein, 17 aa, synthetic construct
  REGION 1..17: note = Synthetic
  VARIANT 1: note = X is C or S
  VARIANT 3: note = X is G or S
  VARIANT 4: note = X is I, T, or V
  VARIANT 7: note = X is A, D, G, or Y
  VARIANT 9: note = X is S or T
  VARIANT 13: note = X is N or S
XIXXGSXIXDYAXWAKG

SEQ ID NO: 227 - protein, 17 aa, synthetic construct
  REGION 1..17: note = Synthetic
  VARIANT 1: note = X is C or S
  VARIANT 5: note = X is G or T
XIFTXSGSTWYANWAKG

SEQ ID NO: 228 - protein, 16 aa, synthetic construct
  REGION 1..16: note = Synthetic
  VARIANT 10: note = X is F or Y
  VARIANT 12: note = X is S or T
SISIGGATYXAXWAKG

SEQ ID NO: 229 - protein, 17 aa, synthetic construct
  REGION 1..17: note = Synthetic
  VARIANT 4: note = X is S or T
  VARIANT 9: note = X is I, S or T
SIYXGSLNXYYATWAKG

SEQ ID NO: 230 - protein, 7 aa, synthetic construct
  REGION 1..7: note = Synthetic
  VARIANT 2: note = X is G or S
  VARIANT 3: note = X is I, T, or V
  VARIANT 6: note = X is A, D, or G
IXXGSXI

SEQ ID NO: 231 - protein, 7 aa, synthetic construct
  REGION 1..7: note = Synthetic
  VARIANT 3: note = X is I or V
IGXGSGI

SEQ ID NO: 232 - protein, 7 aa, synthetic construct
  REGION 1..7: note = Synthetic
  VARIANT 6: note = X is A or D
ISTGSXI

SEQ ID NO: 233 - protein, 7 aa, synthetic construct
  REGION 1..7: note = Synthetic
  VARIANT 4: note = X is G or T
IFTXSGS

SEQ ID NO: 234 - protein, 7 aa, synthetic construct
  REGION 1..7: note = Synthetic
  VARIANT 3: note = X is S or T
IYXGSLN

SEQ ID NO: 235 - protein, 12 aa, synthetic construct
  REGION 1..12: note = Synthetic
  VARIANT 2: note = X is K or R
  VARIANT 5: note = X is A or V
TXDGXGSFYMNL

SEQ ID NO: 236 - protein, 14 aa, synthetic construct
  REGION 1..14: note = Synthetic
  VARIANT 6: note = X is I or S
  VARIANT 10: note = X is D or N
  VARIANT 13: note = X is D or H
  VARIANT 14: note = X is I or T
ARNVDXIYLXAFXX

SEQ ID NO: 237 - protein, 12 aa, synthetic construct
  REGION 1..12: note = Synthetic
  VARIANT 2: note = X is H, N, or T
  VARIANT 5: note = X is A or G
  VARIANT 7: note = X is W or Y
  VARIANT 9: note = X is H or Y.
RXKNXDXGXFDL

SEQ ID NO: 238 - protein, 14 aa, synthetic construct
  REGION 1..14: note = Synthetic
  VARIANT 4: note = X is H, N, or T
  VARIANT 7: note = X is A or G
  VARIANT 9: note = X is W or Y
  VARIANT 11: note = X is H or Y.
ARRXKNXDXGXFDL

SEQ ID NO: 239 - protein, 12 aa, synthetic construct
  REGION 1..12: note = Synthetic
  VARIANT 5: note = X is N, S, or T
  VARIANT 6: note = X is I or V
  VARIANT 8: note = X is N or S
  VARIANT 9: note = X is D or N
  VARIANT 12: note = X is A, C, F, or S
QSSQXXYXXYLX

SEQ ID NO: 240 - protein, 12 aa, synthetic construct
  REGION 1..12: note = Synthetic
  VARIANT 5: note = X is N or T
  VARIANT 12: note = X is F or S
QSSQXIYSDYLX

SEQ ID NO: 241 - protein, 12 aa, synthetic construct
  REGION 1..12: note = Synthetic
  VARIANT 6: note = X is I or V
  VARIANT 8: note = X is N or S
  VARIANT 12: note = X is A, C, or S
QSSQSXYXNYLX

SEQ ID NO: 242 - protein, 11 aa, synthetic construct
  REGION 1..11: note = Synthetic
  VARIANT 4: note = X is G or Q
QASXTIGSNLA

SEQ ID NO: 243 - protein, 11 aa, synthetic construct
  REGION 1..11: note = Synthetic
  VARIANT 4: note = X is E or Q
  VARIANT 7: note = X is G, N, or S
  VARIANT 8: note = X is D, N, or T
  VARIANT 9: note = X is A or R
QASXSIXXXLA

SEQ ID NO: 244 - protein, 11 aa, synthetic construct
  REGION 1..11: note = Synthetic
  VARIANT 4: note = X is E or Q
QASXSINDRLA

SEQ ID NO: 245 - protein, 11 aa, synthetic construct
  REGION 1..11: note = Synthetic
  VARIANT 7: note = X is G or S
  VARIANT 8: note = X is N or T
QASQSIXXALA

SEQ ID NO: 246 - protein, 7 aa, synthetic construct
  REGION 1..7: note = Synthetic
QNIYSDY

SEQ ID NO: 247 - protein, 7 aa, synthetic construct
  REGION 1..7: note = Synthetic
QSIYNNY

SEQ ID NO: 248 - skipped

SEQ ID NO: 249 - protein, 7 aa, synthetic construct
  REGION 1..7: note = Synthetic
  VARIANT 2: note = X is N or T
QXIYSDY

SEQ ID NO: 250 - protein, 7 aa, synthetic construct
  REGION 1..7: note = Synthetic
  VARIANT 3: note = X is I or V
  VARIANT 5: note = X is N or S
QSXYXNY

SEQ ID NO: 251 - protein, 6 aa, synthetic construct
  REGION 1..6: note = Synthetic
  VARIANT 1: note = X is G or Q
XTIGSN

SEQ ID NO: 252 - protein, 6 aa, synthetic construct
  REGION 1..6: note = Synthetic
ESINDR

SEQ ID NO: 253 - skipped

SEQ ID NO: 254 - protein, 6 aa, synthetic construct
  REGION 1..6: note = Synthetic
  VARIANT 1: note = X is E or Q
XSINDR

SEQ ID NO: 255 - protein, 6 aa, synthetic construct
  REGION 1..6: note = Synthetic
  VARIANT 4: note = X is G or S
  VARIANT 5: note = X is N or T
QSIXXA

SEQ ID NO: 256 - protein, 7 aa, synthetic construct
  REGION 1..7: note = Synthetic
  VARIANT 1: note = X is D, G, Q, S, or Y
  VARIANT 2: note = X is A or T
  VARIANT 3: note = X is A or S
XXXTLAS

SEQ ID NO: 257 - protein, 7 aa, synthetic construct
  REGION 1..7: note = Synthetic
  VARIANT 1: note = X is D, G, Q, or Y
  VARIANT 2: note = X is A or T
XXSTLAS

SEQ ID NO: 258 - protein, 7 aa, synthetic construct
  REGION 1..7: note = Synthetic
  VARIANT 2: note = X is A or T
KXFTLAS

SEQ ID NO: 259 - protein, 7 aa, synthetic construct
  REGION 1..7: note = Synthetic
  VARIANT 1: note = X is R or S
  VARIANT 2: note = X is A or S
XXSTLAS

SEQ ID NO: 260 - protein, 7 aa, synthetic construct
  REGION 1..7: note = Synthetic
  VARIANT 1: note = X is A or G
  VARIANT 2: note = X is A or T
XXSTLAS

SEQ ID NO: 261 - protein, 12 aa, synthetic construct
  REGION 1..12: note = Synthetic
  VARIANT 5: note = X is S or T
QQGAXRINIDNA

SEQ ID NO: 262 - protein, 11 aa, synthetic construct
  REGION 1..11: note = Synthetic
  VARIANT 7: note = X is S or T
  VARIANT 9: note = X is I or T
QTYYLTXTXNA

SEQ ID NO: 263 - protein, 12 aa, synthetic construct
  REGION 1..12: note = Synthetic
  VARIANT 10: note = X is D or E
QQGWTVSSLXNA

SEQ ID NO: 264 - protein, 119 aa, synthetic construct
  REGION 1..119: note = Synthetic
  MOD_RES 1: note = Pyroglutamic acid
EEQLEESGGGLVQPEGSLTLTCTASGFSFSSNHYMSWVRQAPGKGLEWIGSIGVGSGITDYASWAKGRFTISKASSTTLTLQMTTLTAADTAIYFCARWTSGGGGFGLWGPGTLVTVSS

SEQ ID NO: 265 - protein, 119 aa, synthetic construct
  REGION 1..119: note = Synthetic
  MOD_RES 1: note = Pyroglutamic acid
EEQLEESGGGLVQPEGSLTLTCTASGFSFSSNHYMCWVRQAPGKGLEWIGCIGVGSGITDYASWAKGRFTISKASSTTLTLQMTTLTAADTAIYFCARWTSGGGGFGLWGPGTLVTVSS

SEQ ID NO: 266 - protein, 119 aa, synthetic construct
  REGION 1..119: note = Synthetic
  MOD_RES 1: note = Pyroglutamic acid
EEQLVESGGGLVQPEGSLTLTCTASGFSFSSAYYMCWVRQAPGKGLEWVGCIGVGSGITDYASWAKGRFTISKTSSTTVTLQMTTLTAADTAIYFCARWTSGGGGFGLWGPGTLVTVSS

SEQ ID NO: 267 - protein, 118 aa, synthetic construct
  REGION 1..118: note = Synthetic
  MOD_RES 1: note = Pyroglutamic acid
EEQLVESGGGLVQPEGSLTLTCTASGFSFSASYYMCWVRQAPGKGLEWIGCIGIGSGITDYANWAEGRFTISKTSTTVTLQMTTLTAADTAIYFCARWTSGGGGFGLWGPGTLVTVSS

SEQ ID NO: 268 - protein, 119 aa, synthetic construct
  REGION 1..119: note = Synthetic
  MOD_RES 1: note = Pyroglutamic acid
EEQLVESGGGLVQPEGSLTLTCTASEFSFSSNYYMCWVRQAPGKGLEWIGCIGIGSYITDYASWAKGRFTISKTSSTTVTLQMTTLTAADTAIYFCARWTSGGGGFGLWGPGTLVTVSS

SEQ ID NO: 269 - protein, 119 aa, synthetic construct
  REGION 1..119: note = Synthetic
  MOD_RES 1: note = Pyroglutamic acid
EEQLVESGGGLVQPEGSLTLTCTASAFSFNNNHYMCWVRQAPGKGLEWIGCIGIGSGISDYANWAKGRFTISKTSSTTVTLQMTTLTAADTAIYFCARWTSGGGGFGLWGPGTLVTVSS

SEQ ID NO: 270 - protein, 119 aa, synthetic construct
  REGION 1..119: note = Synthetic
  MOD_RES 1: note = Pyroglutamic acid
EEQLVESGGGLVQPEGSLTLTCTASEFSFGANYYMCWVRQAPGKGLEWIGCIGIGSGITDYASWAKGRFTISKTSSTTVTLQMTTLTAADTAIYFCARWTSGGGGFGLWGPGTLVTVSS

SEQ ID NO: 271 - protein, 119 aa, synthetic construct
  REGION 1..119: note = Synthetic
  MOD_RES 1: note = Pyroglutamic acid
EEQLVESGGGLVQPEGSLTLTCTASEFSFSNNYYMSWVRQAPGKGLEWLGSIGIGSGITDYASWAKGRFTISKASSTTVTLQMTTLTAADTAIYFCARWTSGGGGFGLWGPGTLVTVSS

SEQ ID NO: 272 - protein, 119 aa, synthetic construct
  REGION 1..119: note = Synthetic
  MOD_RES 1: note = Pyroglutamic acid
EEQLVESGGGLVQPEGSLTLTCTASEFSFSNNYYMCWVRQAPGKGLEWLGCIGIGSGITDYASWAKGRFTISKASSTTVTLQMTTLTAADTAIYFCARWTSGGGGFGLWGPGTLVTVSS

SEQ ID NO: 273 - protein, 119 aa, synthetic construct
  REGION 1..119: note = Synthetic
  MOD_RES 1: note = Pyroglutamic acid
EEQLVESGGGLVQPEGSLTLTCTASEFSFSSNYYMCWVRQAPGKGLEWIGCIGIGSGISDYASWAKGRFTISKASSTTVTLQMTTLTAADTAIYFCARWTSGGGGFGLWGPGTLVTVSS

SEQ ID NO: 274 - protein, 119 aa, synthetic construct
  REGION 1..119: note = Synthetic
  MOD_RES 1: note = Pyroglutamic acid
EEQLVESGGGLVQPEGSLTVTCTASGFDLSRYFYMSWVRQAPGKGLEWIASISTGSDITDYASWAKGRFTISKTSSTTVTLQMTTLTAADTAIYFCARWTSGGGGFGLWGPGTLVTVSS

SEQ ID NO: 275 - protein, 119 aa, synthetic construct
  REGION 1..119: note = Synthetic
  MOD_RES 1: note = Pyroglutamic acid
EEQLEESGGGLVQPEGSLTLTCTASGFDFSSAYWICWVRQAPGKGLEWIGCISTGSAITDYASWAKGRFTISKTSSTTVTLQMTTLTAADTAIYFCARWTSGGGGFGLWGPGTLVTVSS

SEQ ID NO: 276 - protein, 120 aa, synthetic construct
  REGION 1..120: note = Synthetic
  MOD_RES 1: note = Pyroglutamic acid
EEQLEESGGDLVKPEGSLTLTCTASGFSFSSSYYMSWVRQAPGMGLEWIASISGGVTDNAYYASWAKGRFTVSKTSSTTVTLQLTSLTAADTATYFCARDSSSGYFFLLWGPGTLVTVSS

SEQ ID NO: 277 - protein, 118 aa, synthetic construct
  REGION 1..118: note = Synthetic
  MOD_RES 1: note = Pyroglutamic acid
ESVEESGGRLVTPGTPLTLSCTASGFSLSSYYMIWVRQAPGKGLEYIGYVYTGSGNTWYASWAKGRFAISKASTTVDLMITSPTTEDTATYFCASGADGVYDWGWDIWGPGTLVTVSL

SEQ ID NO: 278 - protein, 118 aa, synthetic construct
  REGION 1..118: note = Synthetic
  MOD_RES 1: note = Pyroglutamic acid
ESLEESGGDLVKPGASLTLTCTASGFYFSSGYDMSWVRQAPGKGLEWIASIFTTSGSTWYANWAKGRFTISKTSSTTVTLQMTSLTDADTATYFCTKDGVGSFYMNLWGPGTLVTVSS

SEQ ID NO: 279 - protein, 118 aa, synthetic construct
  REGION 1..118: note = Synthetic
  MOD_RES 1: note = Pyroglutamic acid
ESLEESGGDLVKPGASLTLTCTASGFYFSSGYDMCWVRQAPGKGLEWIACIFTGSGSTWYANWAKGRFTISKTSSTTVTLQMTSLTDADTATYFCTRDGAGSFYMNLWGPGTLVTVSS

SEQ ID NO: 280 - protein, 117 aa, synthetic construct
  REGION 1..117: note = Synthetic
  MOD_RES 1: note = Pyroglutamic acid
ESVEESGGRLVTPGTPLTLTCAVSGFSLSTYALGWVRQAPGKGLEWIGSISIGGATYYASWAKGRFTISKTSTTVDLKIASPTTEDTATYFCARNVDSIYLDAFHTWGPGTLVTVSL

SEQ ID NO: 281 - protein, 117 aa, synthetic construct
  REGION 1..117: note = Synthetic
  MOD_RES 1: note = Pyroglutamic acid
ESVEESGGRLVTPGTPLTLTCTVSGIDLSTYAMGWVRQAPGKGLEWIGSISIGGATYFATWAKGRFTISKGSTTVDLKIASPTTEDTATYFCARNVDIIYLNAFDIWGPGTLVTVSL

SEQ ID NO: 282 - protein, 121 aa, synthetic construct
  REGION 1..121: note = Synthetic
  MOD_RES 1: note = Pyroglutamic acid
EEQLEESGGGLVQPEGSLTLTCTVSGFSFSGDYYMSWVRQAPGKGLEWVASIYTGSLNSYYATWAKGRFTISLTSSTTVTLQMTSLTAADTATYFCARRNKNADYGHFDLWGPGTLVTVSS

SEQ ID NO: 283 - protein, 121 aa, synthetic construct
  REGION 1..121: note = Synthetic
  MOD_RES 1: note = Pyroglutamic acid
EEQLEESGGGLVQPEGSLTLTCTASGFSFSGDYYMSWVRQAPGKGLEWIASIYTGSLNTYYATWAKGRFTISLTSSTTVTLQMTSLTAADTATYFCARRHKNADYGHFDLWGPGTLVTVSS

SEQ ID NO: 284 - protein, 121 aa, synthetic construct
  REGION 1..121: note = Synthetic
  MOD_RES 1: note = Pyroglutamic acid
EEQLEESGGGLVQPEGSLTLTCTASGFSFSDDYYMSWVRQAPGKGLEWIASIYTGSLNTYYATWAKGRFTISLTSSTTVTLQMTSLTAADTATYFCARRNKNADWGHFDLWGPGTLVTVSS

SEQ ID NO: 285 - protein, 121 aa, synthetic construct
  REGION 1..121: note = Synthetic
  MOD_RES 1: note = Pyroglutamic acid
EEHLEESGGGLVQPEGALTLTCTASGFSFSGDYYMSWVRQAPGKGLEWIASIYTGSLNTYYATWAKGRFTISIASSTTVTLQVTSLTAADTATYFCARRTKNGDYGYFDLWGPGTLVTVSS

SEQ ID NO: 286 - protein, 121 aa, synthetic construct
  REGION 1..121: note = Synthetic
  MOD_RES 1: note = Pyroglutamic acid
EEQLEESGGDLVKPEGSLTLTCTASGFSFSGDYYMSWVRQAPGKGLEWIASIYTGSLNIYYATWAKGRFTISLTSSTTVTLQMTSLTAADTATYFCARRNKNADYGHFDLWGPGTLVTVSS

SEQ ID NO: 287 - protein, 121 aa, synthetic construct
  REGION 1..121: note = Synthetic
  MOD_RES 1: note = Pyroglutamic acid
EEQLEESGGGLVQPEGSLTLTCTASGFSFSGDWYMSWVRQAPGKGLEWIASIYSGSLNTYYATWAKGRFTVSLTSSTTVTLQMTSLTAADTATYFCARRNKNADYGHFDLWGPGTLVTVSS

SEQ ID NO: 288 - protein, 121 aa, synthetic construct
  REGION 1..121: note = Synthetic
  MOD_RES 1: note = Pyroglutamic acid
EEQLEESGGGLVQPEGSLTLTCTVSGFSFSGDYYMSWVRQAPGKGLEWVASIYTGSLNTYYATWAKGRFTISLTSSTTVTLQMTSLTAADTATYFCARRNKNADYGHFDLWGPGTLVTVSS

SEQ ID NO: 289 - protein, 121 aa, synthetic construct
  REGION 1..121: note = Synthetic
  MOD_RES 1: note = Pyroglutamic acid
EEQLEESGGGLVQPEGSLTLTCTASGFSFSGDYYMSWVRQAPGKGLEWIASIYTGSLNTYYATWAKGRFTISLTSSTTVTLQMTSLTAADTATYFCARRNKNADYGHFDLWGPGTLVTVSS

SEQ ID NO: 290 - protein, 108 aa, synthetic construct
  REGION 1..108: note = Synthetic
  MOD_RES 1: note = Pyroglutamic acid
EVLTQTSSPVSAAVGGTVTINCQSSQTIYSDYLFWYQQTPGQPPKLLIYQTSTLASGVPSRFKGSGSGTEFTLTISEIQCDDAATYYCQGYYSGYIWTFGGGTEVVVR

SEQ ID NO: 291 - protein, 108 aa, synthetic construct; identical to SEQ ID NO: 290
  REGION 1..108: note = Synthetic
  MOD_RES 1: note = Pyroglutamic acid

SEQ ID NO: 292 - protein, 108 aa, synthetic construct
  REGION 1..108: note = Synthetic
  MOD_RES 1: note = Pyroglutamic acid
EVLTQTASPVSAAVGGTVTISCQSSQSVYSNYLSWYQQKPGQPPKLLIYYASTLASGVPSRFKGSGSGTQFTLTISGVQCDDAATYYCQGYYSGYIWTFGGGTEVVVK

SEQ ID NO: 293 - protein, 108 aa, synthetic construct
  REGION 1..108: note = Synthetic
  MOD_RES 1: note = Pyroglutamic acid
EVLTQTASPVSAAVGSTVTINCQSSQSVYSNYLAWYQQKPGQPPNLLIYGASTLASGVPSRFKGSGSGTQFTLTISDLQCDDAATYYCQGYYSGYIWTFGGGTEVVVK

SEQ ID NO: 294 - protein, 108 aa, synthetic construct
  REGION 1..108: note = Synthetic
  MOD_RES 1: note = Pyroglutamic acid
EVLTQTSSPVSAAVGGTVTINCQSSQTIYSDYLSWYQQKPGQPPKLLIYQTSTLASGVPSRFKGSGSGTEFTLTIREIQCDDAATYYCQGYYSGYIWTFGGGTEVVVK

SEQ ID NO: 295 - protein, 108 aa, synthetic construct
  REGION 1..108: note = Synthetic
  MOD_RES 1: note = Pyroglutamic acid
EVLTQTASPVSAAVGSTVTINCQSSQNIYSDYLSWYQQKPGQPPKLLIYGASTLASGVPSRFKGNGSGTQFTLTISDLQCDDAATYYCQGYYSGYIWTFGGGTEVVVK

SEQ ID NO: 296 - protein, 108 aa, synthetic construct
  REGION 1..108: note = Synthetic
  MOD_RES 1: note = Pyroglutamic acid
EVLTQTSSPVSAAVGGTVTINCQSSQTIYSDYLFWYQQKPGQPPKLLIYQTSTLASGVPSRFSGSGSGTQFTLTISGVQCDDAATYYCQGYYSGYIWTFGGGTEVVVN

SEQ ID NO: 297 - protein, 108 aa, synthetic construct; identical to SEQ ID NO: 296
  REGION 1..108: note = Synthetic
  MOD_RES 1: note = Pyroglutamic acid

SEQ ID NO: 298 - protein, 108 aa, synthetic construct
  REGION 1..108: note = Synthetic
  MOD_RES 1: note = Pyroglutamic acid
EVLTQTSSPVSAAVGGTVTISCQSSQTIYSDYLFWYQQKPGQPPKLLIYQTSTLASGVPSRFKGSGSGTEFTLTISEMQCDDAATYYCQGYYSGYIWTFGGGTEVVVK

SEQ ID NO: 299 - protein, 108 aa, synthetic construct
  REGION 1..108: note = Synthetic
  MOD_RES 1: note = Pyroglutamic acid
EVLTQTASPVSAAVGGTVTLNCQSSQSIYNNYLSWYQQRAGQPPKLLIYSTATLASGVPSRFKGSGSGTQFTLTISEIQCEDAATYYCQGYYSGYIWTFGGGTEVVVK

SEQ ID NO: 300 - protein, 108 aa, synthetic construct
  REGION 1..108: note = Synthetic
  MOD_RES 1: note = Pyroglutamic acid
EVLTQTASPVSAAVGGTVTINCQSSQSVYSNYLCWYQQKPGQPPKLLMYDASTLASGVPSRFKGSGSGTQFTLTISGVQCDDAATYYCQGYYSGYIWTFGGGTEVVVK

SEQ ID NO: 301 - protein, 6 aa, synthetic construct
  REGION 1..6: note = Synthetic
  VARIANT 1: note = X is N or S
  VARIANT 6: note = X is C or S
XNHYMX

SEQ ID NO: 302 - skipped

SEQ ID NO: 303 - protein, 6 aa, synthetic construct
  REGION 1..6: note = Synthetic
  VARIANT 1: note = X is A, N, or S
  VARIANT 2: note = X is A, N or S
XXYYMC

SEQ ID NO: 304 - protein, 6 aa, synthetic construct
  REGION 1..6: note = Synthetic
  VARIANT 6: note = X is C or S
SGYDMX

SEQ ID NO: 305 - protein, 6 aa, synthetic construct
  REGION 1..6: note = Synthetic
  VARIANT 5: note = X is L or M
STYAXG

SEQ ID NO: 306 - protein, 6 aa, synthetic construct
  REGION 1..6: note = Synthetic
  VARIANT 1: note = X is G or D
  VARIANT 3: note = X is W or Y
XDXYMS

SEQ ID NO: 307 - protein, 11 aa, synthetic construct
  REGION 1..11: note = Synthetic
  VARIANT 6: note = X is A or S
  VARIANT 7: note = X is A, N, or S
  VARIANT 8: note = X is H or Y
  VARIANT 11: note = X is C or S
GFSFSXXXYMX

SEQ ID NO: 308 - protein, 11 aa, synthetic construct
  REGION 1..11: note = Synthetic
  VARIANT 5: note = X is G or S
  VARIANT 6: note = X is A, N, or S
  VARIANT 11: note = X is C or S
EFSFXXNYYMX

SEQ ID NO: 309 - protein, 11 aa, synthetic construct
  REGION 1..11: note = Synthetic
  VARIANT 11: note = X is C or S
GFYFSSGYDMX

SEQ ID NO: 310 - protein, 10 aa, synthetic construct
  REGION 1..10: note = Synthetic
  VARIANT 2: note = X is F or I
  VARIANT 3: note = X is D or S
  VARIANT 9: note = X is L or M
GXXLSTYAXG

SEQ ID NO: 311 - protein, 11 aa, synthetic construct
  REGION 1..11: note = Synthetic
  VARIANT 6: note = X is D or G
  VARIANT 8: note = X is W or Y
GFSFSXDXYMS

SEQ ID NO: 312 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 313 - protein, 303 aa, Macaca fascicularis
MRIFAVFIFTIYWHLLNAFTVTVPKDLYVVEYGSNMTIECKFPVEKQLDLTSLIVYWEMEDKNIIQFVHGEEDLKVQHSNYRQRAQLLKDQLSLGNAALRITDVKLQDAGVYRCMISYGGADYKRITVKVNAPYNKINQRILVVDPVTSEHELTCQAEGYPKAEVIWTSSDHQVLSGKTTTTNSKREEKLLNVTSTLRINTTANEIFYCIFRRLDPEENHTAELVIPELPLALPPNERTHLVILGAIFLLLGVALTFIFYLRKGRMMDMKKCGIRVTNSKKQRGKNIRRNWEVEGKGNKKLKQ